(19) United States
(12) Patent Application Publication
(10) Pub. No.: US 2021/0158895 A1
(43) Pub. Date: May 27, 2021

(54) ULTRA-SENSITIVE DETECTION OF CANCER BY ALGORITHMIC ANALYSIS

(71) Applicant: Dana-Farber Cancer Institute, Inc., Boston, MA (US)

(72) Inventor: Scott L. Carter, Cambridge, MA (US)

(21) Appl. No.: 17/047,320

(22) PCT Filed: Apr. 15, 2019
(86) PCT No.: PCT/US19/27525

§ 371 Date: Oct. 13, 2020

Related U.S. Application Data
(60) Provisional application No. 62/827,044, filed on Mar. 30, 2019.
(60) Provisional application No. 62/658,891, filed on Apr. 17, 2018.
(60) Provisional application No. 62/657,606, filed on Apr. 13, 2018.

(51) Int. Cl. G16B 20/20; G16B 20/10; G16B 40/20; G16H 10/40; C12Q 1/6886

(52) U.S. Cl. CPC G16B 20/20; G16B 20/10; C12Q 2600/156; G16H 10/40; C12Q 1/6886; G16B 40/20

(57) ABSTRACT

Ultra-sensitive detection of cancer by algorithmic analysis. In various embodiments, a sample comprising a plurality of polynucleotides is analyzed. A plurality of sequences of the plurality of polynucleotides is received. The plurality of sequences is provided to a trained classifier. The trained classifier is adapted to accept a plurality of sequences and output a class label indicative of the presence of a somatic variant within the plurality of sequences. A class label is received from the trained classifier indicative of the presence of a somatic clone within the plurality of sequences.

### CROSS-REFERENCE TO RELATED APPLICATIONS

This application claims the benefit of U.S. Provisional Application No. 62/657,606, filed Apr. 13, 2018, U.S. Provisional Application No. 62/658,891, filed Apr. 17, 2018, and U.S. Provisional Application No. 62/827,044, filed Mar. 30, 2019, which are hereby incorporated by reference in their entireties.

### BACKGROUND

Embodiments of the present disclosure relate to whole-genome sequencing, and more specifically, to ultra-sensitive detection of cancer by algorithmic analysis.

### BRIEF SUMMARY

According to embodiments of the present disclosure, methods of and computer program products for analyzing samples comprising a plurality of polynucleotides are provided. In various embodiments, a plurality of sequences of the plurality of polynucleotides is received. The plurality of sequences is provided to a trained classifier. The trained classifier is adapted to accept a plurality of sequences and output a class label indicative of the presence of a somatic variant within the plurality of sequences. A class label indicative of the presence of a somatic clone within the plurality of sequences is received from the trained classifier.

In some embodiments, based on the label, it is indicated whether a cancer clone is present in the sample. In some embodiments, based on the label, it is indicated whether a clonal expansion is present in the sample. In some embodiments, the label has an associated probability.

In some embodiments, clinical data are provided to the trained classifier, the clinical data being related to an originator of the sample. In some embodiments, the clinical data comprise an indication of smoking by the originator of the sample. In some embodiments, the clinical data comprise family history of the originator of the sample. In some embodiments, population data are provided to the trained classifier, the population data being related to an originator of the sample.

In some embodiments, the plurality of sequences comprise a plurality of somatic variants.

In some embodiments, trained classifier comprises an artificial neural network. In some embodiments, the trained classifier comprises a regression model. In some embodiments, the trained classifier comprises a random decision forest. In some embodiments, the trained classifier comprises an SVM. In some embodiments, the neural network is a convolutional neural network. In some embodiments, the artificial neural network is a recurrent neural network.

In some embodiments, the sample comprises blood. In some embodiments, the sample comprises cerebrospinal fluid. In some embodiments, the plurality of polynucleotides comprises DNA. In some embodiments, the plurality of polynucleotides comprises methylated DNA. In some embodiments, the plurality of polynucleotides comprises RNA.

In some embodiments, fragment lengths of the plurality of sequences are provided to the trained classifier.

In some embodiments, the sequencing is at a depth of 100× or less. In some embodiments, the sequencing is at a depth of 85× or less. In some embodiments, the sequencing is at a depth of about 20× to about 85×. In some embodiments, the sequencing is at a depth of about 20× to about 100×. In some embodiments, the plurality of polynucleotides is sequenced to obtain the plurality of sequences.

According to embodiments of the present disclosure, methods of and computer program products for analyzing samples comprising a plurality of polynucleotides are provided. In various embodiments, at least one prior sequence of a polynucleotide associated with a tumor genome is received. A generative model is fitted to the at least one prior sequence. A plurality of sequences of the plurality of polynucleotides is received. The generative model is applied to the plurality of sequences to determine a probability that a first somatic clone is present in the plurality of sequences. Based on the probability, a label indicative of the presence of the first somatic clone in the sample is determined.

In some embodiments, the label has an associated probability. In some embodiments, the generative model comprises a linear-Gaussian model. In some embodiments, the generative model comprises a linear-negative binomial model. In some embodiments, the generative model comprises a latent factor model. In some embodiments, the generative model comprises a factor analysis model.

In some embodiments, a phylogenetic tree is inferred from the at least one prior sequence. In some embodiments, the generative model is updated based on the plurality of sequences. In some embodiments, the at least one prior sequence comprises a second somatic clone, and based on the updated generative model, a probability that the sample contains the second somatic clone is determined.

In some embodiments, the at least one prior sequence comprises a second somatic clone, and, based on the updated generative model, a probability that the sample contains a descendent of the second somatic clone is determined. In some embodiments, the at least one prior sequence comprises a second somatic clone, and, based on the updated generative model, a probability that the sample contains a third somatic clone related to the second somatic clone within the phylogenetic tree is determined. In some embodiments, based on the updated generative model, a probability that the sample shares at least one somatic mutation with the at least one prior sequence is determined. In some embodiments, based on the updated generative model, a probability that the sample shares at least one clonal expansion with the at least one prior sequence is determined.

In some embodiments, based on the label, it is indicated whether a cancer clone is present in the sample. In some embodiments, based on the label, it is indicated whether a clonal expansion is present in the sample. In some embodiments, the label has an associated probability. In some embodiments, the plurality of sequences comprise a plurality of somatic variants.

In some embodiments, the sample comprises blood. In some embodiments, the sample comprises cerebrospinal fluid. In some embodiments, the plurality of polynucleotides comprises DNA. In some embodiments, the plurality of polynucleotides comprises methylated DNA. In some embodiments, the plurality of polynucleotides comprises RNA.

In some embodiments, the sequencing is at a depth of 100× or less. In some embodiments, the sequencing is at a depth of 85× or less. In some embodiments, the sequencing is at a depth of about 20× to about 85×. In some embodiments, the sequencing is at a depth of about 20× to about 100×. In some embodiments, the plurality of polynucleotides is sequenced to obtain the plurality of sequences.

According to embodiments of the present disclosure, methods of and computer program products for analyzing samples comprising a plurality of polynucleotides are provided. In various embodiments, a plurality of sequences of the plurality of polynucleotides is received. One or more inherited variant and one or more somatic variant among the plurality of sequences are identified. The one or more inherited variant is provided to a first trained classifier. The one or more somatic variant is provided to a second trained classifier. The presence of aneuploidy in the plurality of polynucleotides is determined by the first and second trained classifier.

In various embodiments, the plurality of sequences comprise a plurality of somatic variants. In various embodiments, the first or second trained classifier comprises an artificial neural network. In various embodiments, the first or second trained classifier comprises a regression model. In various embodiments, the first or second trained classifier comprises a random decision forest. In various embodiments, the first or second trained classifier comprises an SVM. In various embodiments, the artificial neural network is a convolutional neural network. In various embodiments, the artificial neural network is a recurrent neural network.

In various embodiments, the sample comprises blood. In various embodiments, the sample comprises cerebrospinal fluid. In various embodiments, the plurality of polynucleotides comprises DNA. In various embodiments, the plurality of polynucleotides comprises methylated DNA. In various embodiments, the plurality of polynucleotides comprises RNA.

In various embodiments, the sequencing is at a depth of 100× or less. In various embodiments, the sequencing is at a depth of 85× or less. In various embodiments, the sequencing is at a depth of about 20× to about 85×. In various embodiments, the sequencing is at a depth of about 20× to about 100×. In various embodiments, the plurality of polynucleotides is sequenced to obtain the plurality of sequences.

### BRIEF DESCRIPTION OF THE SEVERAL VIEWS OF THE DRAWINGS

FIGS. 1A-B are plots of the expected number of unique reads as a function of raw sequencing depth for cfDNA samples.

FIG. 2 illustrates sensitive detection of cancer in cfDNA using reference-tumor somatic copy number alterations (SCNAs) according to embodiments of the present disclosure.

FIGS. 3A-C depict power calculations for cancer detection using a typical lung-cancer genome as a tumor reference genome.

FIGS. 4A-F illustrate improved detection of allelic imbalance in highly impure tumor DNA samples according to embodiments of the present disclosure.

FIG. 5 illustrates the power to detect de novo subclinical cancer using mutational signature analysis according to embodiments of the present disclosure.

FIGS. 6A-C illustrate exemplary integrative analysis of genomic copy-number and point mutation according to embodiments of the present disclosure.

FIG. 7 illustrates a method of analyzing a sample comprising a plurality of polynucleotides according to embodiments of the present disclosure.

FIG. 8 illustrates a method of analyzing a sample comprising a plurality of polynucleotides according to embodiments of the present disclosure.

FIGS. 9A-B provide data illustrating robust detection of subclinical lung cancer in a patient according to embodiments of the present disclosure.

FIGS. 10A-E are 2D scatter plot of exemplary variant-allele fraction according to embodiments of the present disclosure.

FIGS. 11A-C provide data illustrating robust detection of breast cancer in a patient according to embodiments of the present disclosure.

FIGS. 12A-O are 2D scatter plots of exemplary variant-allele fraction according to embodiments of the present disclosure.

FIGS. 13A-AD are 2D scatter plots of exemplary variant-allele fraction according to embodiments of the present disclosure.

FIG. 14A is a graph of the expected number of tracking variants detected against cancer fraction according to embodiments of the present disclosure.

FIG. 14B is a graph of detection power against cancer fraction according to embodiments of the present disclosure.

FIG. 15 depicts a computing node according to an embodiment of the present disclosure.

### DETAILED DESCRIPTION

Early detection is one of the most effective ways to increase the cure rate for cancer, which remains the second leading cause of death in the United States. One approach to non-invasive early cancer detection is to perform genomic analysis of cell-free DNA (cfDNA), also known as liquid biopsy. Since genome sequencing of plasma cfDNA from routine blood draws can be used to detect the presence of DNA shed from tumors, this approach holds the potential to make early cancer detection a routine clinical procedure.

Genomic analysis of plasma cell free DNA (cfDNA) is useful due to its ability to non-invasively assay potential cancer-vulnerabilities in metastatic patients. Plasma cfDNA genotyping has a high positive predictive value (PPV) for detecting driver mutations in oncogenes like EGFR and KRAS, making it useful as a tool for detecting driver mutations and guiding use of targeted therapies. As discussed above, the fundamental limitation of plasma genotyping is that sensitivity for detecting known tumor-related mutations is imperfect. For example, CT-imaging can discern lung nodules as small as 0.034 cm³, corresponding to an expected cancer-cfDNA fraction of 1.4×10⁻⁴ (95% CI: 6.4×10⁻⁶-3.1×10⁻³) whereas the current limit of detection for targeted genotyping is approximately 1/1,000.

Clinically meaningful increases in detection sensitivity can be realized using the WGS approaches described herein. Among patients with advanced EGFR-mutant non small-cell lung cancer (NSCLC), rapid reductions in plasma mutations can be seen in the initial months on therapy, followed by re-emergence prior to radiographic disease progression. Some metastatic lung cancers shed very little tumor DNA into the plasma, resulting in false negatives even with highly sensitive assays. This highlights the imperfect sensitivity of alternative PCR-based plasma genotyping assays, which are unable to detect circulating tumor DNA in patients with advanced NSCLC having a response to therapy.

Several alternative technical approaches may be taken to achieve improved sensitivity, including ultradeep sequencing and personalized (or bespoke) sequencing assays. Bespoke assays aim to increase sensitivity through PCR amplification of specific selector regions where mutations exist in a patient's cancer. Applicability of such an approach is limited by the need to sequence the tumor and then develop personalized PCR primers for detection of tumor DNA, a logistically complex process which might impair routine clinical use. Ultradeep sequencing assays aim to increase sensitivity through deep sequencing of key regions of the genome. Such an approach can be costly, is inherently limited in breadth, and may not improve sensitivity given the low levels of circulating DNA present in patients with a low burden of cancer.

The limited DNA in input material imposes a fundamental physical limitation on detection. Accordingly, targeted PCR or deep-coverage NGS assays are unlikely to achieve useful sensitivity for patients with subclinical cancer. Such patients will typically shed only a few nanograms of cfDNA per tube of blood—the mass equivalent of approximately 1,000 genomes (3.6 ng)—so the resolution to quantify any given mutation cannot be greater than 1 in 1,000, even assuming error-free sequencing. In practice, this limitation is made even more severe by the limited efficiency (10-30%) of adaptor ligation prior to PCR amplification of DNA for NGS, resulting in loss of the majority of DNA fragments during library preparation.

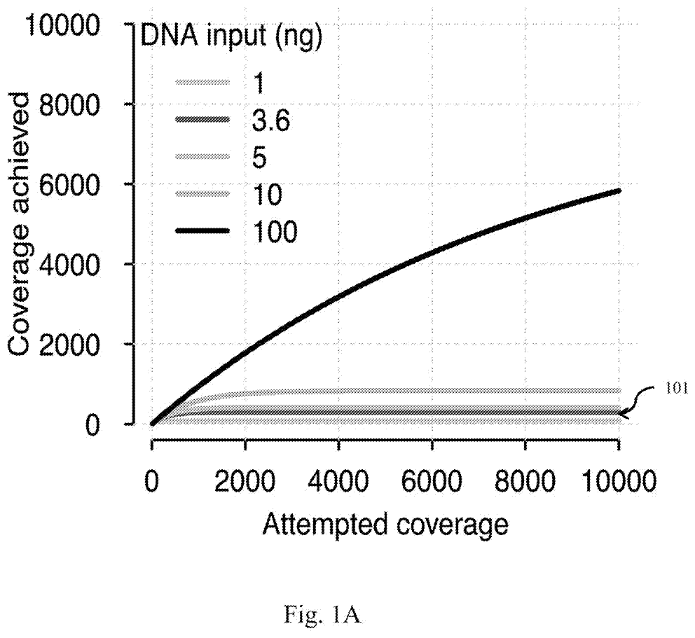

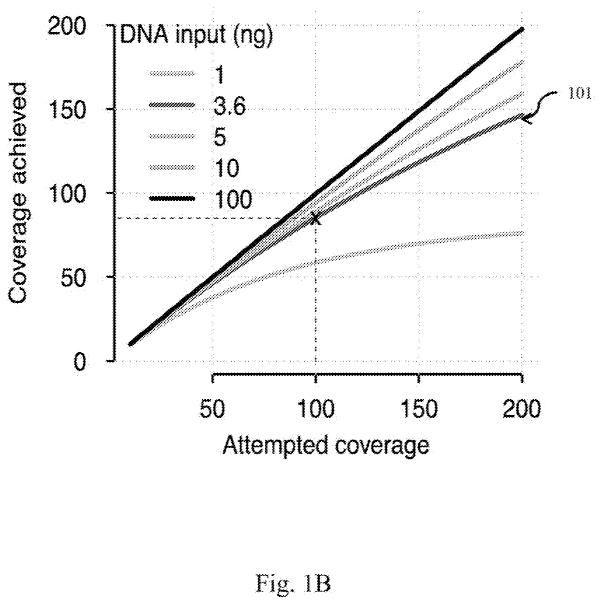

Referring to FIGS. 1A-B, plots are provided of the expected number of unique reads as a function of raw sequencing depth, illustrating the diminishing returns on increasing attempted coverage when using limited-input cfDNA samples. The y-axis shows the expected number of unique reads (true coverage) as a function of raw sequencing depth attempted (x-axis). Highlighted curve 101 corresponds to an input DNA mass of 3.6 ng, a typical quantity obtainable from plasma cfDNA in a patient with subclinical cancer. FIG. 1B is a zoomed in view of FIG. 1A, showing that moderate-depth coverage is possible with limited DNA input. Assuming 30% adaptor-ligation efficiency, then 100× attempted coverage will yield 85× de-duplicated (true) coverage (dashed lines).

In general, coverage (or depth) in DNA sequencing is the number of reads that include a given nucleotide in the reconstructed sequence. Deep sequencing refers to the general concept of aiming for high number of replicate reads of each region of a sequence. Even though the sequencing accuracy for each individual nucleotide may be very high, the very large number of nucleotides in a genome means that if an individual genome is only sequenced once, there will be a significant number of sequencing errors. Many positions in a genome contain rare single-nucleotide polymorphisms (SNPs).

A further limitation of targeted-sequencing approaches is that that they are unable to distinguish benign from malignant clones, since many indolent clonal expansions harbor driver mutations identical to those found in deadly cancers. All somatic tissues result from an evolutionary process, whereby repeated rounds of mutation and selection result in mosaic genetic heterogeneity. Although cancer can be thought of as an extreme case involving strong positive selection and clonal expansion, such expansions have also been demonstrated in physiologically normal tissues, and are likely pervasive. Distinguishing benign from malignant clones requires methods that can incorporate genome-wide clonal co-variation to sensitively track clonal populations and match them to population-level knowledge banks.

To address these and other shortcomings of alternative approaches, various embodiments of the present disclosure leverage breadth of genomic coverage, rather than depth, to achieve ultra-sensitive detection of somatic clonal expansions consistent with cancer. Whole genome sequencing (WGS) to moderate depth (20-100×) can provide excellent sensitivity to detect the presence of somatic cancer clones by statistical integration of weak evidence from up to millions of somatic alterations, even though the sensitivity to detect any single cancer mutation is low. WGS approaches described herein address the challenge of clonal heterogeneity by statistical reconstruction of phylogenetic trees relating all somatic clones detected in a given patient. Longitudinal tracking of clones, rather than mutations, enables discrimination between benign (slow growing) and malignant (fast growing) clones; even if both harbor identical drivers, they may be distinguished by their unique passenger mutations, of which hundreds to millions may be revealed by WGS.

The calculations depicted in FIG. 1 illustrate that various approaches described herein enable a transformational sensitivity increase of up to three orders of magnitude over alternative approaches, and thus, improve early detection of a range of cancers. The approaches described herein may be applied with existing whole-genome sequencing (WGS) of plasma-based cfDNA obtained from peripheral blood collected during the course of clinical care. It will be appreciated that, in addition, the present disclosure is applicable to any other polynucleotides that may be sequenced by WGS techniques, including DNA, RNA, methylated DNA, or isolated fragments thereof.

As set out herein, clonal phylogeny inference may be applied to low-purity longitudinal cfDNA sequencing data. In this way, improved signal-to-noise ratios is achieved in somatic DNA copy-number and mutation calling. Various embodiments leverage clonal deconvolution and statistical reconstruction of phylogenetic trees relating all somatic clones detected in a given patient.

In various embodiments, a flexible statistical analysis toolkit is provided to detect minute de novo somatic clones. Methods are provided for blinded detection of somatic clones consistent with cancer. Various embodiments apply Bayesian statistics to integrate evidence from signatures of aneuploidy and mutation that are learned from population-level analyses of TCGA datasets. These may be adapted to the risk profile of the patient being monitored.

The statistical approaches to WGS provided herein transform plasma-based genotyping liquid biopsy from a tool used for guiding palliative therapy of metastatic cancer into diagnostics that can be used to detect subclinical cancer and impact cure rates in early-stage, curable cancers. In various embodiments, the techniques described herein are useful to:
Identify patients who would benefit from additional adjuvant therapy to prevent or delay clinical recurrence based on the presence of residual cancer-derived cfDNA following therapy with curative intent.
Measure the extent of a patient's response to immune-checkpoint blockade therapy, where imaging is inherently ambiguous due to the pseudo-progression phenomenon, whereby inflammation due to therapeutic efficacy can be indistinguishable from tumor progression—a major problem in clinical decision-making.
Enable precision interception of cancer in healthy individuals by ultrasensitive blood-based cfDNA screening and monitoring. Approaches are provided to tailor such screening to an individual's risk profile as determined by family history, germline risk alleles, and environmental exposures.
Study the clonal dynamics of cancer during therapy using liquid biopsy to sample disease states at high temporal resolution—such studies can reveal the cancer subclones sensitive to a therapy and provide an early readout of efficacy as well as insights into genetic drivers of resistance and/or sensitivity. Ultrasensitive readout of clonal fractions at each timepoint allows these techniques to be applied in cases where the overall cancer-cfDNA shed is very low or when the change in clonal fraction between time-points is very small.

The statistical methods provided herein outperform alternative methods for accurate somatic variant calling from next-generation sequencing data, and thus have broad applicability to cancer genome characterization in virtually all contexts. The probabilistic methods provided herein can be directly applied to various sequencing strategies, including whole-exome sequencing, targeted clinical gene panels, or whole-genome sequencing. This confers enormous flexibility to design cost-effective studies and encourages widespread adoption.

The novel computational methods provided herein for fully automated clonal deconvolution and phylogenetic reconstruction are broadly applicable to the study of intra-tumor heterogeneity and evolution. This is particularly true for studies based on cfDNA and/or archival formalin-fixed paraffin-embedded (FFPE) blocks, for which single-cell sequencing will never be an option.

Whole-genome sequencing (WGS) to moderate depth (20-100×) can provide excellent sensitivity to detect the presence of somatic cancer clones, even though the sensitivity to detect any single cancer mutation is low. This is because the probabilities of various cancer-genome alterations being represented in a sample of cfDNA are statistically independent of one another. Therefore, sensitive detection can be achieved by statistical integration of weak evidence from up to millions of somatic alterations. This approach can realize a transformational sensitivity increase of at least three orders of magnitude over alternative approaches.

In various embodiments, deep learning techniques are used to dramatically improve the sensitivity and specificity for distinguishing true point mutations from sequencing artifacts. Linear-Gaussian dimensionality reduction is used in some embodiments to remove noise from genomic copy-ratio profiles.

In various embodiments, joint estimation of allelic ratios at heterozygous SNP sites across all samples from a given patient is performed, which can be combined with panel-based SNP phasing using the Baum-Welch algorithm for hidden Markov models to achieve unprecedented sensitivity to identify allelic imbalance due to aneuploidy—a hallmark of cancer genomes.

In various embodiments, multi-sample inference of clonal phylogeny is provided. Methods are provided to statistically infer cancer phylogenies from mixed DNA sequencing data.

Ultrasensitive phylogenetic monitoring using reference tumor-genome is provided. Such embodiments enable optimal exploitation of all available information about a patient's cancer genome in order to increase sensitivity to detect recurrence.

Ultrasensitive detection of de novo somatic clones is provided without use of a reference tumor-genome, based on integrating signals from aneuploidy and mutational signatures associated with cancer. These can be tuned to a patient's personal risk profile based on family history and/or environmental exposures.

As set out below, in various embodiments, methods for clonal phylogeny inference are applied to low-purity longitudinal cfDNA sequencing data. All somatic tissues result from an evolutionary process, whereby repeated rounds of mutation and selection result in mosaic genetic heterogeneity. Computational methods may be used to deconvolve complex DNA sequencing data obtained from genetically heterogeneous cancer tissue-samples in order to infer tumor content, DNA ploidy, whole-genome doubling, absolute copy-numbers, and subclonal alterations.

Statistical clonal-deconvolution is successful due to fact that aneuploidy is an early and pervasive event in many cancers. Most human tumor samples are parsimoniously explained by a single dominant clone discernable by aneuploidy, often containing smaller nested copy-number and sequence variants. This pattern has been confirmed by single-cell DNA sequencing, revealing nearly indistinguishable copy-number profiles among the 1,000s of primary-tumor cells sequenced. This observation is also highly consistent with classic models of tumorigenesis. Although human cancer tissues are genetically heterogeneous, discrete clonal phylogenies can be readily reconstructed using statistical analysis of bulk cancer-tissue DNA sequencing data. It follows that multiple samples of cancer-tissue from the same patient provide increased power to discern tumor clonal substructure and lessen the reliance on parsimony.

In various embodiments, robust and general Bayesian methods are provided. Such approaches intrinsically operate on all available samples from a given patient through all stages of analysis, including processing of raw read-count data to call somatic variants, resolution of clonal populations, and inference of phylogenetic relationships. Various approached herein cope with inherent ambiguity in the data when only a small number of patient-samples are available for inference, such as in TCGA or clinical sequencing. Many inferences of interest, such as whether a given point-mutation is homozygous, can still be made reliably even when other aspects of the sample(s) are not uniquely identified. In addition, these methods naturally gain more power for clonal reconstruction as more samples from a given cancer are sequenced.

Methods for robust multi-sample inference of somatic variation and clonal phylogeny can be extended to enable a transformative increase in the sensitivity at which subclinical cancer may be detected.

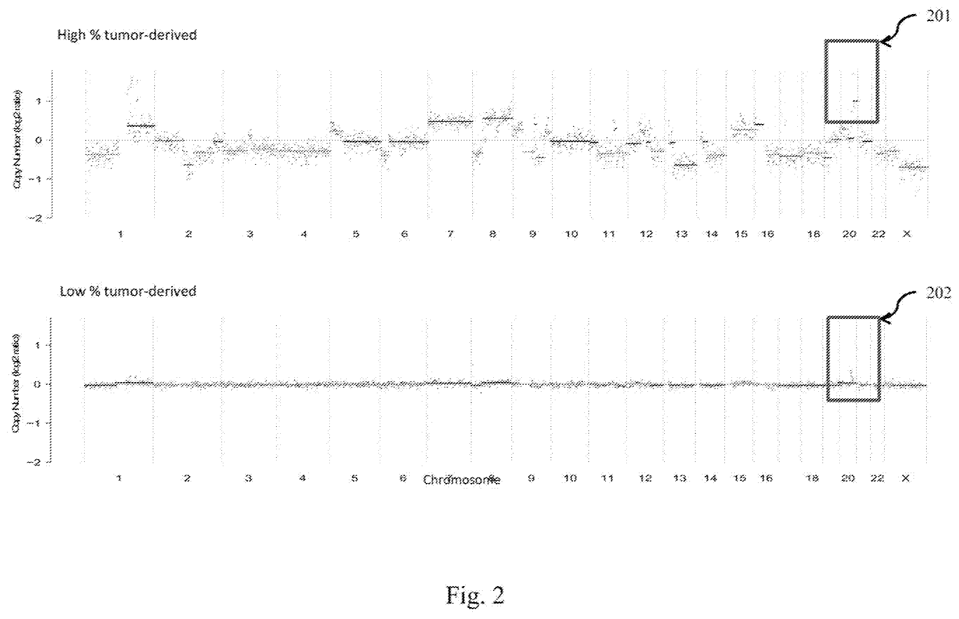

Referring to FIG. 2, sensitive detection of cancer in cfDNA using reference-tumor somatic copy number alterations (SCNAs) detected in WGS data is illustrated. Rectangles 201, 202 highlight a high-level focal amplicon visually apparent in both samples. cfDNA was collected from the cerebro-spinal fluid (CSF) and plasma at the same clinical visit. The patient had active lepto-meningeal disease, but no active extracranial disease.

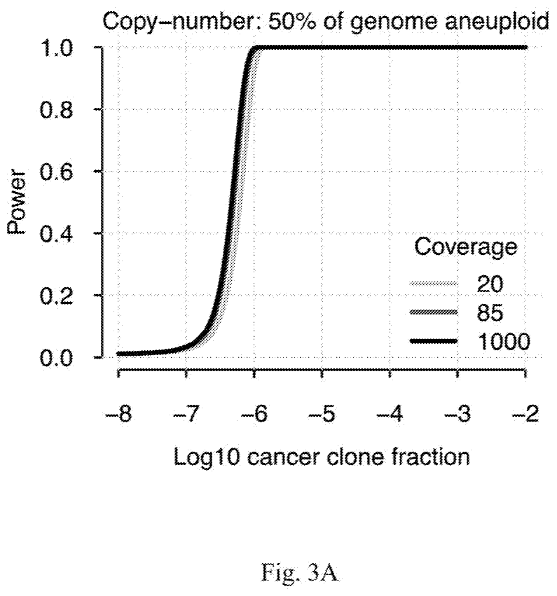

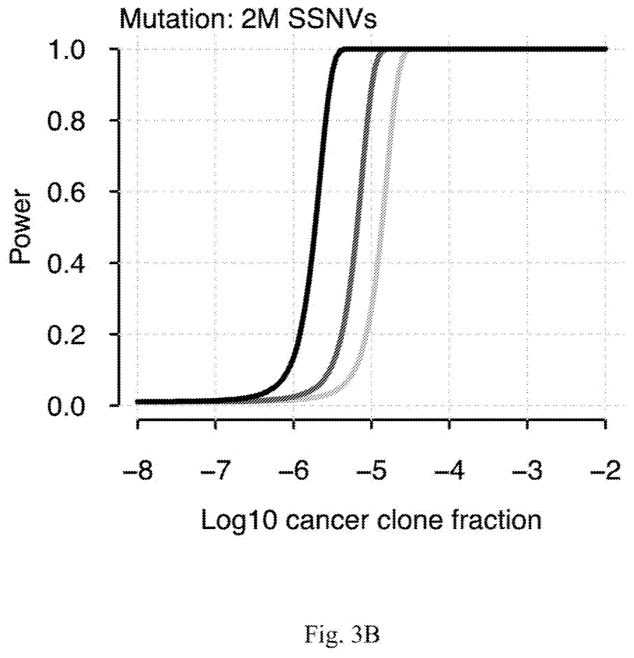

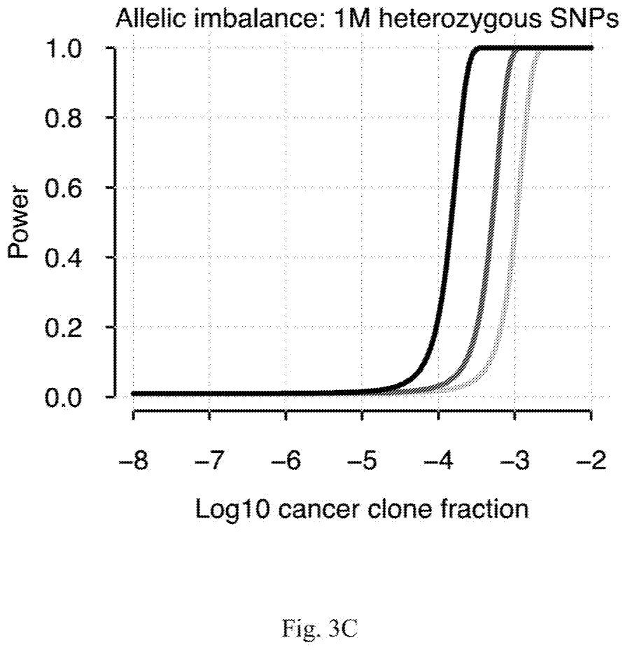

Referring to FIGS. 3A-C, power calculations for cancer detection using a typical lung-cancer genome as a tumor reference genome are provided. All calculations assume a sequencing error-rate of 1 in 1,000 and control the false positive rate at 1%. In FIG. 3A, SCNAs (50% of the genome aneuploid at 1-copy imbalance), Log copy-ratios were modeled at 1 kb resolution as Gaussian with variance 0.15, similar to typical values obtained from WGS data. In FIG. 3B, Mutations (2 million SSNVs) are illustrated. In FIG. 3C, Allelic imbalance at SNPs is illustrated, an indicator of aneuploidy.

Anecdotal data with clinical specimens as illustrated in FIG. 2, as well as simulation data as illustrated in FIG. 3 demonstrate that multi-sample inference of somatic variation and clonal phylogeny can be used with moderate-depth (20-85×) WGS using low-input DNA, and can detect low-level tumor DNA in settings where ultra-deep targeted sequencing would be inefficient. Alternative approaches to deal with cancer heterogeneity other than the statistical deconvolution methods are mostly inapplicable to cfDNA samples, since they utilize (i) single-cell sequencing data; (ii) complex structural genomic variants, which will not be well powered for detection in cfDNA samples due to fact that cfDNA is sheared into short fragments of 166 bp; or (iii) long-range haplotype-resolved reads, also not compatible with short cfDNA fragments.

In various embodiments, denoising total copy-ratio profiles with linear-Gaussian dimensionality reduction is provided. Denoising algorithms are applied for improved estimation of somatic copy-ratios from DNA sequencing depth. These methods extends probabilistic principal component analysis to model library size, using a generative linear-Gaussian model whereby each sample is projected onto a linear combination of latent bias factors, each having a mean and precision for each genomic region.

In various embodiments, multi-sample inference of allelic ratios is provided that operate on multiple patient-samples simultaneously. This is statistically more powerful the single sample approaches, since all samples from a given patient must share the same SNP genotypes. Thus, even DNA samples that contain almost no tumor may be assessed accurately for allelic imbalance, provided at least one high-purity tumor-sample is available to learn the SNP phases.

In various embodiments, multi-sample inference of clonal phylogeny is provided. A phylogenetic framework is provided that regularizes model complexity by leveraging biological assumptions regarding clonal evolution and phylogeny (e.g., most mutations occur once during evolution and are clonally transmitted to all descendants, other than in cases of mutation loss by segmental deletion). These assumptions are highly consistent with plausible models of somatic clonal evolution and have demonstrated broad empirical support in numerous cancer datasets. This model thus has greater statistical power and confers robustness to sequencing artifacts, which are unlikely to be consistent with a well-resolved phylogeny. Furthermore, as a result of jointly modeling all samples from a given patient together, more power is gained as more patient-samples are sequenced, since more complex phylogenies can gain definitive support.

Inference of tumor purity and ploidy is provided that operates on multiple tissue samples simultaneously. Such methods infer the number of distinct somatic clones k represented in the s samples sequenced, as well as the clonal phylogeny relating them (represented as a k×k matrix T, where T_{i,j}=1 if clone i is an ancestor of clone j, and 0 otherwise), and the clonal fractions in each sample (represented as an s×k matrix P, where the rows sum to 1). In addition, the segmental somatic copy-numbers in each clone are represented as a k×n matrix D, where n is the number of segments. The first row of D, corresponding to the germline, is set to 2 in all segments other than regions of copy-number polymorphism or sex chromosomes, and subsequent rows (clones) contain the integer change in copy-number at that clone/segment.

The following matrix equation is obtained for total copy-ratio (tCR): tCR=ΨPPTD. Ψ is an s×s diagonal matrix of nuisance factors: the inverse of the average ploidy of all clones in each sample, weighted by their clonal fractions. This factor, is necessary to convert mixed integer copy-numbers into copy-ratios, reflecting the fact that a constant mass of DNA library is input for sequencing, so sequencing depth reflects locus concentration, rather than absolute count. The above equation along with the copy-ratio error-model allows inference of P, T, and D. Furthermore, this equation can be extended to also incorporate allelic-fractions at each segment (using allelic depth at heterozygous SNPs), as well as the multiplicity of somatic mutations in each clone. Because each of these data-sources are independent and measured with defined error, a combined likelihood can be calculated, allowing integration of evidence from each source of data available for a given cancer. Algorithms are provided to fit this model using a hybrid approach incorporating greedy search and importance sampling.

In various embodiments, ultrasensitive phylogenetic monitoring is provided using reference tumor-genome. In order to optimally leverage prior knowledge of a patient's tumor genome to enable ultrasensitive detection, the model above is fit using all available patient samples with an appreciable amount of cancer-derived cfDNA (e.g., pre-operative high-shed cfDNA samples, or else sampled cancer tissue). A low-shed cfDNA sample may then be analyzed using this phylogenetic model of the patient's cancer simply by adding a new row to P for the new sample. This leaves only k−1 free parameters to optimize (the fraction of each somatic clone present in the new sample), and optimally utilizes all of the copy-ratio, allelic ratio, and mutational profiles of all known clones. The probability of cancer being present in the sample is equivalent to the probability than any of the k−1 somatic clonal fractions is >0.

Because cancers are likely to continue evolving during monitoring, this can be extended to increase the weight of evidence from variants at internal branches of the cancer's clonal phylogeny. This could increase sensitivity since those variants are more likely to be present at relapse. One advantageous property of this approach is that, as more samples are sequenced, the more resolved the phylogeny will become, and detection power will increase monotonically. This approach can deliver clinically transformative increases in sensitivity, as illustrated in FIG. 3.

As set out above, optimal statistical methods are provided for multi-sample somatic variant calling and clonal phylogenetic reconstruction. Many samples undetectable using ddPCR (˜1/1,000) sensitivity will be detectible using the WGS approaches provided herein. Because WGS of cfDNA alone may not be adequately powered to detect all cryptic cancer clones of potential interest, aberrant methylation patterns may be incorporated by performing whole-genome bisulfide sequencing (WGBS).

In various embodiments, a flexible statistical analysis toolkit to detect minute de novo somatic clones is provided. For cases where no reference tumor-genome exists at appreciable fraction, such as cancer screening, techniques are provided for maximally sensitive detection of de novo somatic clones consistent with the presence of cancer. These techniques leverage the genomic properties of cancer—such as common mutational processes and/or pervasive aneuploidy—in order to achieve sensitive discrimination of cancer clones from sequencing artifacts in the largest number of samples. As in the monitoring case discussed above, the fact that millions of somatic variants may be independently observed using WGS is leveraged to achieve sensitivity. Several statistical approaches are provided to this problem, that can achieve high sensitivity.

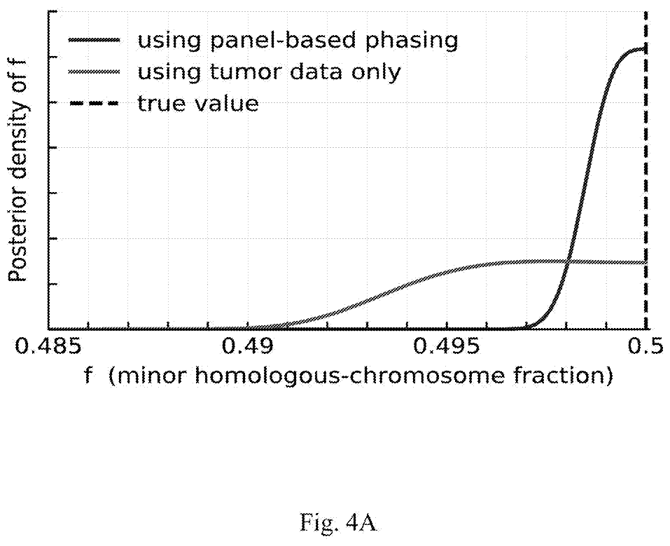

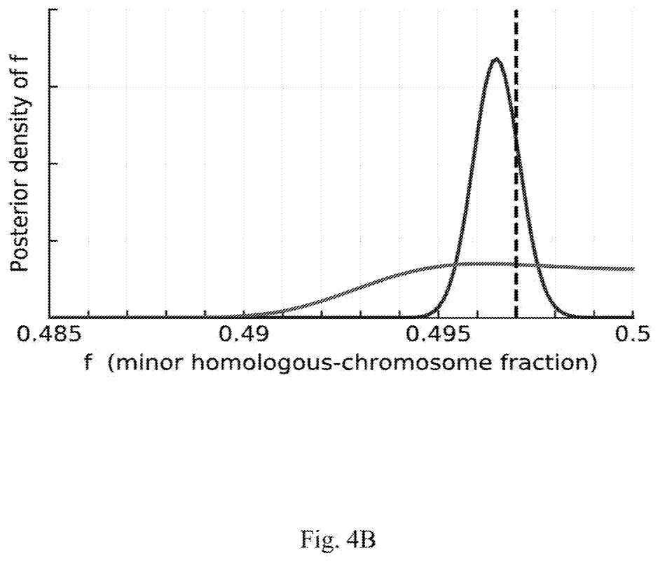

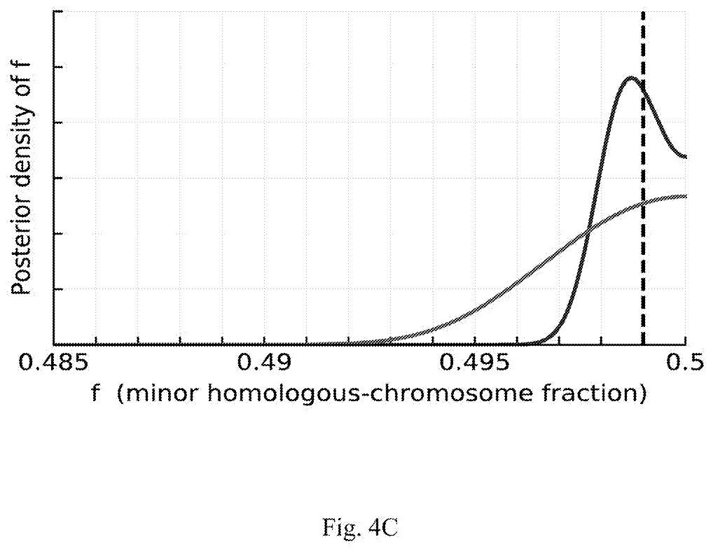

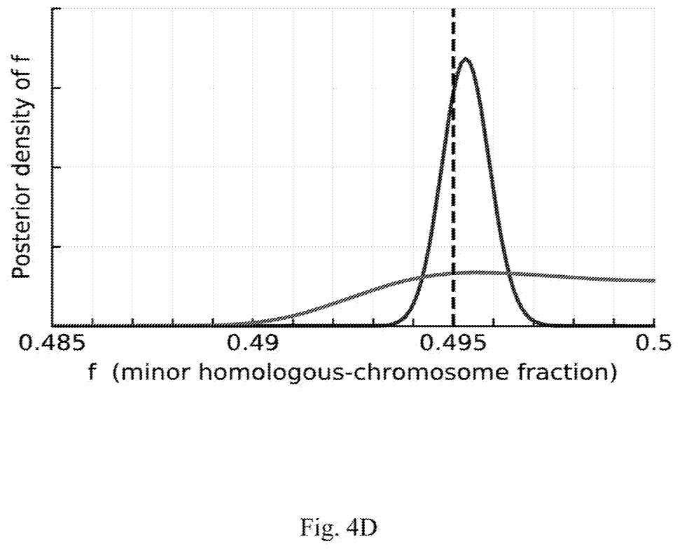

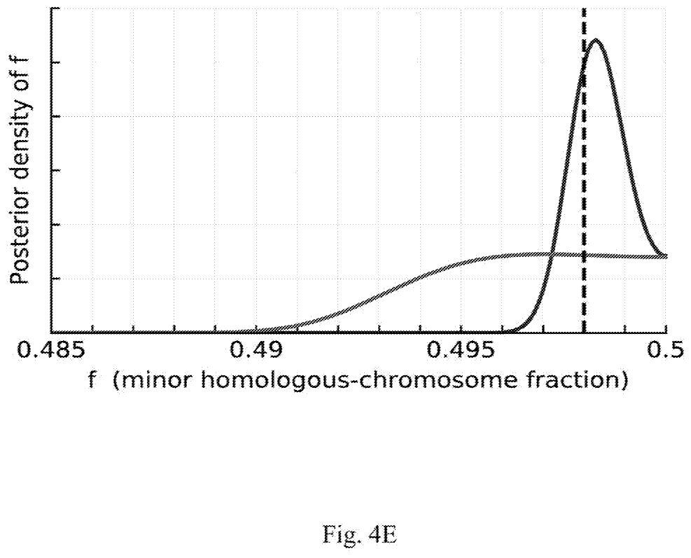

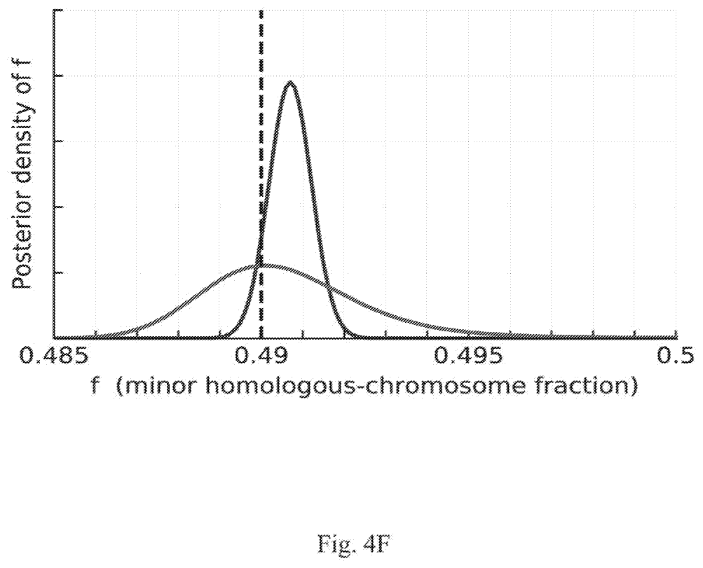

Referring to FIGS. 4A-F, improved detection of allelic imbalance in highly impure tumor DNA samples by combining panel-based statistical phasing with cfDNA-based phasing is illustrated. Curves show inferred posterior probability density functions over the quantity f, the fraction of DNA derived from the minor homologous chromosome. Results obtained using only observed imbalances in allelic depth at heterozygous SNP sites to infer segmental allelic imbalance are compared with those obtained by combining panel-based haplotype phasing with tumor allelic depth. The combined approach yields densities substantially more concentrated at the true f values (dashed vertical lines). This effect is especially pronounced as f approaches 0.5 (perfect allelic balance). These results were obtained by simulating 100× coverage of a genomic segment containing 10,000 heterozygous SNP sites and a probability of panel-based switch-error of 1 in 1,000 SNPs.

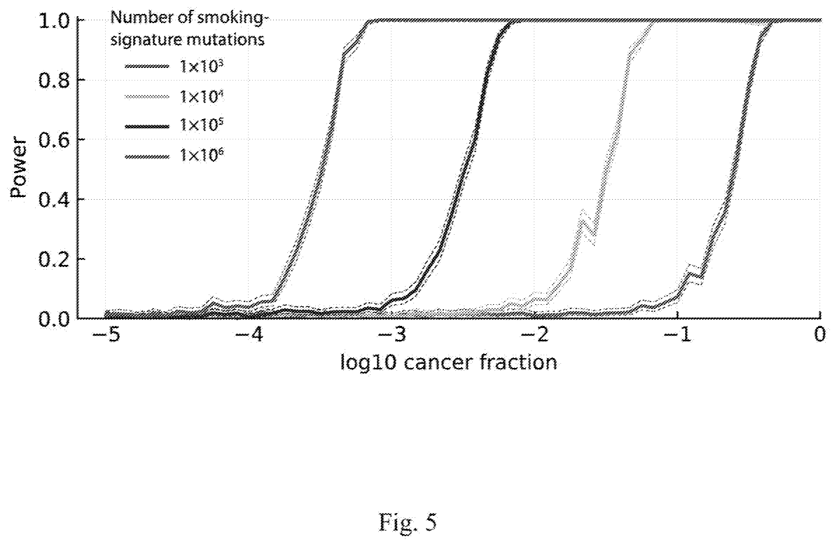

Referring to FIG. 5, power to detect de novo subclinical cancer using mutational signature analysis is illustrated. Even without a paired tumor tissue or high-shed cfDNA sample, low-level cancer clones can still be identified at clinically relevant sensitivity using Bayesian methods for mutational signature analysis. This will allow them to be applied in a cancer-screening setting, or when no reference tumor-genome is available. Results are based on simulated 100× whole-genome sequencing with an error-rate of 1 in 1,000 sequenced bases (with 300M total spurious variants due to sequencing-errors expected in the data). The indicated number of mutations generated by the smoking signature were simulated. Each point on the curves was generated by repeating the simulation 1,000 times and computing the fraction of runs where cancer was detected. The detection threshold was selected by holding the false-positive rate fixed at 0.01. Dashed lines around each curve represent 99% credible intervals.

As set out herein, even without a reference tumor genome, sensitive detection of cancer in cfDNA is possible using statistical approaches. A flexible and robust statistical analysis toolkit is provided to detect minute somatic clones consistent with subclinical cancer in cfDNA samples using moderate depth whole-genome sequencing. In order to optimally integrate evidence from multiple classes of genomic alterations so that sensitivity is maximized, statistical methods are provided integrating evidence from multiple cancer-associated mutational processes using a hierarchical Bayesian model.

In various embodiments, ultrasensitive detection of de novo somatic clones using aneuploidy is provided. Aneuploidy is a hallmark of nearly all cancers, and is pervasive in epithelial cancers. Statistical frameworks are provided to use somatic copy number alterations (SCNAs) in two ways to detect de novo clonal expansions: (a) by the resulting imbalance in the allelic depth—the number of reads supporting the alternate (variant) and reference allele at heterozygous SNPs (hets), and (b) by the effect on the total number of reads at each locus, quantified in terms of total copy-ratio (tCR).

The allelic imbalance model may be described as follows. At het h, the number of alt reads is distributed as aₕ˜Binomial(Nₕ, fₕ), where Nₕ is the total read count (alt+ref) and fₕ is the fraction of copies in the sample that contain the alt allele. In particular, in copy neutral regions, aₕ˜Binomial(Nₕ, 1/2). More generally, for all hets within a region of constant copy number, fₕ is either ϕ or 1−ϕ depending on the phase, where ϕ or 1−ϕ are the fractions of copies of that region coming from each homologous chromosome. To model this, a time-inhomogeneous hidden Markov model (HMM) with a Binomial emission distribution is used, with the hidden state representing both the phase and the value of ϕ. For each cancer type t, an HMM is defined with a transition matrix sequence informed by previous data on commonly amplified/deleted regions, and informed by a patient-specific estimate of phase using a panel-based haplotype-phasing method such as SHAPEIT2. Maximum likelihood estimates of the set of ϕ values are computed using the Baum-Welch algorithm. For a patient with alt read counts a=(a₁, . . . , a_{H}) at hets h=1, . . . , H, the marginal likelihood p(a|t) of cancer type t is computed using a specialized Laplace approximation, summing over all possible sequences of hidden states and integrating over the ϕ parameters. Total log copy-ratios r=(r₁, . . . , r_{B}) for bins b=1, . . . , B can be incorporated into the HMM using a Gaussian emission model.

Combining tumor allelic-imbalance with panel-based phasing dramatically improves the concentration of the posterior distribution over f as illustrated in FIG. 4, demonstrating that this technique improves sensitivity to confidently identify aneuploid genomic segments in cryptic somatic clones. We will further extend these methods to incorporate prior information from genetic recombination maps tailored to the inferred ancestry of the input sample. Such information can be incorporated into our MINI by increasing the state-transition probability corresponding to switch-errors in regions of high meiotic recombination.

In various embodiments, ultrasensitive detection of de novo somatic clones using mutational signatures is provided. The idea behind this technique is to examine all possible genomic variants, even if they have only a single supporting sequencing read. In the case of mutations, this is expected to produce an enormous number of false positives: 3 billion bases×100× coverage×1/1,000 error-rate=300M errors expected. Detection leverages the fact that many lung cancer genomes associated with tobacco smoking will be heavily mutagenized by a well-established mutational process that mutates DNA bases in a highly non-random manner easily appreciated by considering the base substitution within its 5′ and 3′ sequence context.

Specifically, each candidate somatic single-nucleotide substitution (SSNV) can be classified according to the bases involved in the substitution and the bases immediately adjacent on the 5′ and 3′ sides, e.g., _{5′}ACG_{3′}>_{5′}AGG_{3′}, resulting in 96 such classes of SSNV. Different types of cancer exhibit different relative proportions of these SSNV classes. Further, these proportions can be decomposed into characteristic signatures or frequency profiles (w₁ₖ, w₂ₖ, . . . , w_{96,k}) corresponding to common mutational processes k=1, . . . , K.

The number of SSNVs of each class is well modeled by a Poisson distribution—specifically, xᵢ˜Poisson (Σₖ₌₁^{K}wᵢₖθₖ) where xᵢ denotes the number of SSNVs of class i in the sample, and θₖ represents the patient's exposure to mutational process k.

Novel statistical methods are provided to leverage this information to detect cancerous clonal expansions using the presence of signatures that are highly consistent with cancer. More precisely, a Bayesian approach is taken where for each cancer type t a targeted model is defined specifying a subset of active mutational processes and a prior distribution p(θ|t) on the patient's exposure to each active process, which may be informed by covariates such as known environmental exposure or familial history. The marginal likelihood of cancer type t is then p(x|t)=∫p(x|θ)p(θ|t)dθ for a patient with SSNV substitution class counts x=(x₁, . . . , x₉₆). Although exact computation of these integrals is prohibitively complex, a fast algorithm using the Laplace approximation may be applied. This approximation may be validated using Markov chain Monte Carlo (MCMC) sampling.

Power calculations as shown in FIG. 5 demonstrate the utility of this approach for early detection of lung cancer. Variable numbers of mutations from the smoking process are simulated, as well as 300M errors. For realistic numbers of smoking-associated mutations (between 100K and 1M), this technique alone is powered to detect cancer clones as small as 1/1,000 shedding cells as shown in FIG. 5. This technique may be applied to mutational signatures (or combinations thereof) other than tobacco exposure. This technique may be combined with other genomic analyses (for, e.g., aneuploidy) to improve sensitivity.

To evaluate the evidence for clonal expansions, various embodiments use a Bayesian approach to integrate information from all of these models. A patient-specific prior probability of each cancer type t is defined based on covariates and any previous test data that may be available for the patient. The posterior probability is computed of each cancer type t—as well as the null model of no cancer (t=0)—using Bayes' formula: p(t|x, a, r)∝p(x|t)p(a, r|t)p(t), modeling the SSNV class counts x as conditionally independent of the het alt read counts a and the total copy ratios r, given t. From this, the posterior probability of the presence of a cancer clone can easily be computed as Σ_{t≠0}p(t|x, a, r).

In various embodiments, filtering of sequencing artifacts is provided using deep learning. One of the challenges with somatic mutation analysis in the ultrasensitive detection paradigm is that many somatic variants appear at low allele fractions (even a single supporting read), making it difficult to distinguish them from sequencing errors. In practice, this is made even worse because the lack of adequate base-specific error-models from cfDNA WGS data requires an assumption that the per-site error-rate is the genome-wide average of 1/1,000, even though many sites have lower error, and some have more. Furthermore, various properties of the sequencing reads can inform error-probabilities, meaning that even genome-wide base-level models will not be optimal for error suppression.

A bank of filters may be used for read-covariates and a site-level panel-of-normals (PoN). In such approaches, a filter removes potential calls based on a single attribute the filter is designed to detect. However, such filters are hard and do not consider other covariates. If one filter detects something mildly suspicious, but the potential variant passes all other filters easily, it is still removed. Additionally, such filters are generally developed off of general human observations, which may not identify more subtle tells. Another issue is that all the filters are tweaked to work well with a specific kind of sequencer and other sequencers may have a completely different error space. A deep learning model addresses these problems of filter-based approaches.

In various embodiments, a neural network classifier is applies. A neural network learns useful covariates, provided the model is deep enough, allowing the network to detect patterns that may be indicative of sequence errors that humans aren't going to notice. Other benefits include a reduced reliance on a panel of normal genome sequences, which serves to detect rare kinds of errors that aren't covered by filters in the old model. If a neural network is trained well it has no need for such a panel, since it should have learned ways to identify these kinds of errors.

Suitable artificial neural networks include but are not limited to a feedforward neural network, a radial basis function network, a self-organizing map, learning vector quantization, a recurrent neural network, a Hopfield network, a Boltzmann machine, an echo state network, long short term memory, a bi-directional recurrent neural network, a hierarchical recurrent neural network, a stochastic neural network, a modular neural network, an associative neural network, a deep neural network, a deep belief network, a convolutional neural networks, a convolutional deep belief network, a large memory storage and retrieval neural network, a deep Boltzmann machine, a deep stacking network, a tensor deep stacking network, a spike and slab restricted Boltzmann machine, a compound hierarchical-deep model, a deep coding network, a multilayer kernel machine, or a deep Q-network.

Various examples provided herein use a neural network classifier. However, it will be appreciated that a variety of other classifiers are suitable for use according to the present disclosure, including random decision forest, linear classifiers, support vector machines (SVM), or neural networks such as recurrent neural networks (RNN).

In an exemplary embodiment, a deep neural network model learns on the sequence context surrounding a potential somatic variant, as well as some upstream MuTect annotations and summary statistics obtained from the reads at the site of a potential variant. The network is designed to initially process the positional information of the sequence context separately from the read annotations. The sequence context surrounding the potential variant is converted to a 1-hot encoding and then fed through a series of 1-dimensional convolution layers. The goal is to have this section learn specific sequence motifs that can provide information on how to classify a potential variant. The read annotations are fed in as a normalized vector and processed through a series of dense layers. The goal is to have this section learn relationships between annotations that can be useful in classifying a potential variant. The information from these upstream sections of the network is merged and processed through more dense layers. This allows the network to learn relationships between annotations and sequence motifs. The information is then passed through a softmax function producing a vector of length two with elements that sum to one. The first element of the vector is the probability the variant provided is real and the second element is the probability that it is a sequencing artifact. When training the network this output is then given to a binary cross-entropy loss function along with the actual label of the variant, which allows the network to be trained through back-propagation. After the network is trained the greater of the two values identifies how the potential variant is classified.

In an exemplary implementation, gold-standard variant calls from DREAM challenge batches 1 and 3 are used for training and its performance is tested on batches 2 and 4. An accuracy of 97.12% is achieved with a true-positive rate (TPR) of 96.02% and a false-positive rate of 1.79%. This approach therefore has great promise in increasing our ability to sensitively identify cryptic de novo cancer clones using mutational signatures.

The methods described herein may be evaluated by performing in-silico mixing of WGS data from tumor-normal pairs generated, for example, from the Cancer Genome Atlas (TCGA). Aligned reads from tumor-normal BAM pairs may be mixed in variable defined fractions. The resulting mixtures may be analyzed as set forth herein to infer the fraction of tumor DNA present—both using the pure tumor genome as a reference, and using the de novo detection techniques. These mixtures allow generation of validation data for these methods over mixing fractions that might be difficult or expensive to achieve in the laboratory.

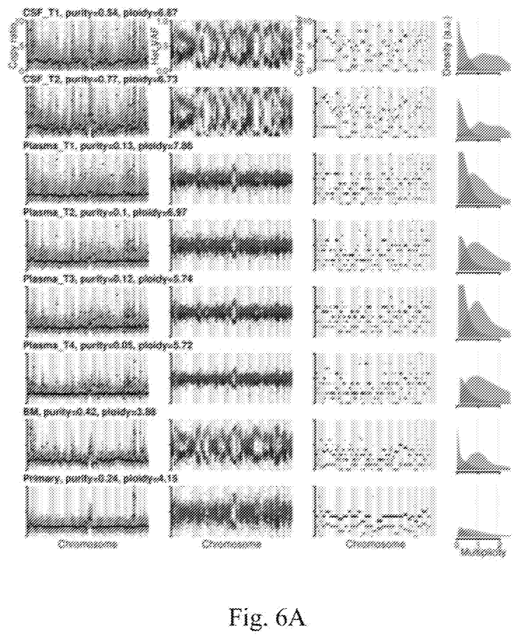

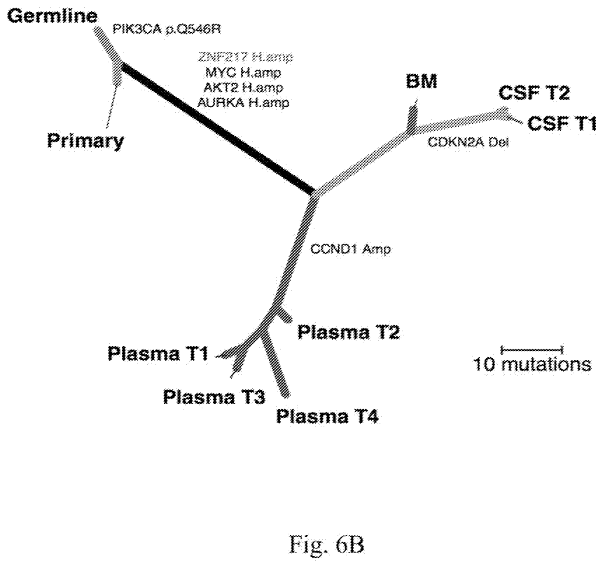

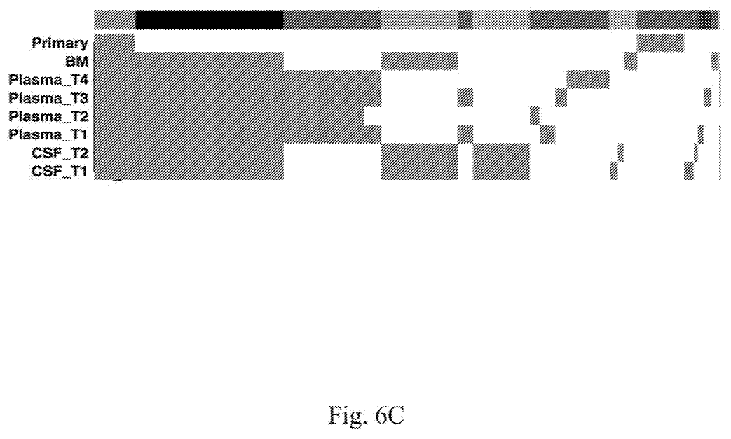

Referring now to FIG. 6, integrative analysis of genomic copy-number and point mutation data across 8 samples is illustrated. In this example CSF, plasma, brain metastasis, and primary tumor samples are drawn from the same patient and subjected to whole-exome sequencing. In the 1st column of FIG. 6A, inference of genome-wide copy-ratios from sequencing data is shown. The black horizontal line indicates segmented copy-ratio values. Colored points show the raw data, with color alternating between orange and blue for adjacent segments. The 2nd column shows inference of allelic copy-ratios from sequencing data. Variant allele fraction (VAF; y-axis) is plotted at all heterozygous SNP sites. Points are colored according to whether they fall on the major (red) or minor (allele). Purple points are in regions of equal allelic copy-numbers. The 3rd column shows inference of absolute allelic copy-number from sequencing data. Horizontal bars indicate absolute copy-numbers (adjusted for sample purity and ploidy). Black corresponds to total copy-number. Pink corresponds to minor-allele copy number. The 4th column shows rescaling of VAF of somatic point mutations to units of multiplicity: the number of alleles per cell. This rescaling adjusts for sample's purity and ploidy and allows subclonal mutations to be identified and quantitated. Green corresponds to mutations present in 100% of the cancer cells in the sequenced sample. Pink corresponds to subclonal mutations present in <100% of cancer cells. In FIG. 6B a phylogenetic tree is depicted derived from these same samples showing that the cfDNA from CSF samples was more related to the brain metastasis, and was divergent from plasma samples and the primary tumor. FIG. 6C shows a matrix of SSNVs (columns) by samples (rows) showing the evidence for the inferred tree.

As described herein, cfDNA is used as a biomarker for residual disease after definitive therapy, or as a pre-diagnostic test to screen for early-stage lung cancers. However, the imperfect sensitivity of existing assays represents a huge roadblock for these pursuits. If 20% of metastatic NSCLC are undetectable in plasma cfDNA (particularly those involving the lungs only), assay sensitivity will likely be inadequate as a screening tool for early stage curable lung cancers.

The use of liquid biopsies as a window to the genetic evolution of central nervous system (CNS) metastases has significant implications for the diagnosis and monitoring of these patients. The genetic drivers of central nervous system (CNS) metastases differ from their matched primary tumors and extracranial metastases in ways that impact clinical decision-making. The results of whole-exome sequencing was conducted in order to construct a global phylogenetic tree relating all cancer subclones detected in each patient. These analyses revealed stark divergence between inter- and extra-cranial disease. This finding presents a major barrier for the use of plasma-based liquid biopsy to effectively inform treatment decisions in patients with CNS metastases, since the cancer clones represented in plasma may by highly divergent from those in the CNS. Furthermore, in patients without extra-cranial disease, plasma may contain only very small amounts of cfDNA, only a small fraction of which is likely to be cancer-derived. Unfortunately, lumbar puncture to collect CSF is an invasive procedure and may not be possible for most patients with CNS metastases. Thus, the capability to access CNS tumor genetics via a peripheral blood (or some other readily accessed biospecimen) would extend the potential benefit of precision targeting of therapy to CNS cancer.

The ultra-sensitive detection methods described herein may be applied to WGS data from paired plasma-CSF cfDNA samples in order to better understand anatomic partitioning of cancer subclones in CNS metastasis.

In the below discussion of an exemplary Allelic CapSeg model, the following terminology is used. For observed data (per segment), a denotes the number of reads supporting alternate base at each SNP and n denotes the total number of reads at each SNP. Model parameters (segment level) includes f∈[0-0.5], the fraction of minor homologous chromosome and τ≥0, the total copy-ratio. The minor homologous chromosome copy-ratio is denoted f_{τ}. The major homologous chromosome copy-ratio is denoted (1−f)τ. f and τ remain independent, even conditionally on the data, unlike SNP arrays. This simplifies inference. The simplest model for the probability of aᵢ alternate reads at SNP i given f is formed from a mixture of 2 binomial distributions:

$$Pr ( {a}_{i} \vert f , {n}_{i} ) = \frac{1}{2} ( Binom ( {a}_{i} \vert f , {n}_{i} ) + Binom ( {a}_{i} \vert ( 1 - f ) , {n}_{i} ) ) \qquad Equation 1$$

The two binomial mixtures represent two possible phases to observe heterozygous SNPs: in phase 1 (alt minor) and phase 2 (ref minor). Using Bayes' rule, this can be inverted to obtain an expression for the likelihood off given data for all the SNPs on the segment:

$$Pr ( f \vert a , n ) ∝ {\prod }_{i} ( \frac{1}{2} ( Binom ( {a}_{i} \vert f , {n}_{i} ) + Binom ( {a}_{i} \vert ( 1 - f ) , {n}_{i} ) ) ) Pr ( f \vert ℋ ) \qquad Equation 2$$

Outliers may also be observed, which can be modeled using a third mixture distribution (uniform on unit interval), having weight ε:

$$Pr ( f \vert a , n ) ∝ {\prod }_{i} ( \frac{1 - ɛ}{2} ( Binom ( {a}_{i} \vert f , {n}_{i} ) + Binom ( {a}_{i} \vert ( 1 - f ) , {n}_{i} ) ) + ɛ ) Pr ( f \vert ℋ ) \qquad Equation 3$$

Adding an outlier component is important to make inferences on f robust to outliers (e.g., genotyping errors). In an exemplary embodiments, ε=0.005.

The quality of this model may be evaluated using normal (germline) samples, where f=0.5.

The simple Binomial sampling model may be further improved by introducing a sample-level model parameter, s_{f}, The average value by which SNPs are skewed in this sample. For example, observed AF=s_{f}×true AF.

This model may be further improved, as can be seen by a quantile-quantile plot of P-values for the SNPs against their expected quantities under the null distribution. The model is inflated, having an excess of highly significant SNPs (lambda=1.34).

The model may be improved by adding an additional sample level parameter v. This parameter allows specification of additional overdispersion using a Beta-Binomial model. Lower values imply more weight in the tails of the distribution, meaning that it expects to see more SNPs with skewed allelic fractions even when f=0.5. This parameter can be interpreted as the number of prior pseudo-observations in the sense implied by the conjugate relationship between the binomial density and with a Beta prior distribution over the true value of p. Adding this parameter allows a much better fit on normal samples, with many values of lambda near 1.0.

In general, binomial sampling of X successes inn trials with probability of success p is given as:

$$X \sim Bin ( n , p ) , the n \\ P ( X = k \vert p , n ) = L ( p \vert k ) = ( \begin{matrix}n \\ k\end{matrix} ) {{p}^{k} ( 1 - p )}^{n - k} \qquad Equation 4$$

In Bayesian modeling, p is considered as a random variable with a prior distribution:

$$\begin{matrix}π ( p \vert α , β ) = Beta ( α , β ) \\ = \frac{{{p}^{α - 1} ( 1 - p )}^{β - 1}}{B ( α , β )}\end{matrix} \qquad Equation 5$$

This leads to the analytically tractable composition integral to obtain the predictive distribution:

$$\begin{matrix}f ( k \vert n , α , β ) = {\int }_{0}^{1} L ( p \vert k ) π ( p \vert α , β ) dp \\ = ( \begin{matrix}n \\ k\end{matrix} ) \frac{1}{B ( α , β )} {\int }_{0}^{1} {{p}^{k + α - 1} ( 1 - p )}^{n - k + β - 1} dp \\ = ( \begin{matrix}n \\ k\end{matrix} ) \frac{B ( k + α , n - k + β )}{B ( α , β )} .\end{matrix} \qquad Equation 6$$

Under maximum entropy assumptions, this distribution represents the optimal integration of information relevant to predict the next (n+1)th trial after observing n prior trials with α successes and β failures. Notice that the above quantity may be calculated directly, without the need for grid or numerical optimization methods—this makes it very easy to use.

To make the optimal inferences regarding the true allelic fraction f in the context of expected bias favoring observation of the reference allele, this distribution is adapted to represent uncertainty about observed a and n values equivalent to having information about f from v observations with α₁ alternate and β₁ reference allele counts in the case of SNPs in phase 1 (alt minor):

α₁=s_{f}fν,

β₁=(1−s_{f}f)ν. Equation 7

and analogous counts α₂ and β₂ for phase 2 (ref minor):

α₂=(1−s_{f}⁻¹f)ν,

β₂=s_{f}⁻¹fν. Equation 8

We then obtain:

$$Pr ( f \vert a , n , {s}_{f} , v ) ∝ ℒ ( f = 0.5 \vert a , n , {\hat{s}}_{f} , \hat{v} ) Pr ( f \vert ℋ ) = {\prod }_{i} ( \frac{1 - ɛ}{2} ( Beta - Binom ( {a}_{i} \vert {α}_{1} , {β}_{1} , {n}_{i} ) + Beta - Binom ( {a}_{i} \vert {α}_{2} , {β}_{2} , {n}_{i} ) ) + ɛ ) Pr ( f \vert ℋ ) \qquad Equation 9$$

If s_{f}=1 then in the limit of ν→00 this is equivalent to the above Binomial model.

Before jointly fitting all segment level parameters τ and f, along with the two sample level parameters s_{f} and ν, the simpler case of normal samples may be considered, where it may be assumed that that τ=1 and f=0.5 for all segments. s_{f} and ν are therefore jointly optimized using coordinate ascent with a numerical optimizer to obtain ŝ_{f} and \hat{ν}.

Fitting this model in ˜100 normal exomes, there is a strong correlation between ŝ_{f} and log \hat{ν}. The quality of this fit indicates that model fitting could be simplified by estimating only ŝ_{f} from the data and then estimating log \hat{ν} by inserting ŝ_{f} into a linear model: log \hat{ν}=âŝ_{f}+\hat{b}, with â and \hat{b} learned in a panel of normal exomes.

This simplification facilitates model fitting in cancer samples, where segment level parameters τ and f must also be fit. Because f and the remaining sample level parameter s_{f} are conditionally independent of τ, they may be optimized separately using coordinate ascent with a numerical optimization.

Even in an aneuploid tumor sample, many segments will be present at allelic balance, that is, both homologous chromosomes have the same copy-ratio. In these cases, f=0.5. However, random fluctuations in the observed data may cause ML estimates of f to be <0.5. From a Bayesian perspective, for a given segment, the free parameter f may not be a justified addition to the simpler model where f=0.5.

We write the evidence for the two models, this quantity represents the extent to which the model fits the data, averaged over all configurations of free parameters weighted by their probability given the data (posterior) multiplied by their prior distributions. For the allelic balance model, there is no free parameter f at the segment level, and the evidence is equivalent to the above data likelihood with f=0.5:

Pr(a,n| [symbol] ₀)= [symbol] (f=0.5|a,n, [symbol] ,\hat{ν}). Equation 10

In the case of the more complex model [symbol] ₁, this evidence calculation requires integration over the posterior distribution of the free parameter f:

$$Pr ( a , n \vert {1}_{} ) = {{\int }_{0}}^{0.5} ℒ ( f \vert a , n , , \hat{v} ) Pr ( f \vert {1}_{} ) df \qquad Equation 11$$

A uniform prior density is specified on f over 0 to 0.5, resulting in Pr(f| [symbol] ₁)=2. Considering [symbol] ₀ and [symbol] ₁ to be the only possible models, Bayes' rule may be applied to obtain:

$$Pr ( {0}_{} \vert a , n ) = \frac{Pr ( a , n \vert {0}_{} ) Pr ( {0}_{} )}{Pr ( a , n \vert {0}_{} ) Pr ( {0}_{} ) + Pr ( a , n \vert {1}_{} ) Pr ( {1}_{} )} , \qquad Equation 12$$

and Pr ( [symbol] ₁|a, n)=1−Pr( [symbol] ₀|a, n). Equal prior probabilities for the two models are specified:

$$Pr ( {0}_{} ) = Pr ( {1}_{} ) = \frac{1}{2} .$$

In DNA, there are four bases: cytosine (C), thymine (T), adenine (A), and guanine (G). Cytosine always pairs with guanine, and thymine always pairs with adenine. Thus, distinguishing one of the two strands of a given DNA molecule, there are four possible base pairs at each point: C-G, G-C, T-A, and A-T.

When considering base pair substitutions at a given point, the convention is to distinguish the strand containing the pyrimidine (C or T) before the substitution has been made. (Cytosine and thymine are pyrimidines, whereas adenine and guanine are purines.) With this convention, there are six possible types of substitutions at any given point:

TABLE 1

| | before | after |
|---|---|---|
| 1 | C-G | A-T |
| 2 | C-G | G-C |
| 3 | C-G | T-A |
| 4 | T-A | A-T |
| 5 | T-A | C-G |
| 6 | T-A | G-C |

Sometimes, these are abbreviated as C>A, C>G, C>T, T>A, T>C, and T>G, denoting only the pre-substitution pyrimidine and what it changes to.

These six classes can be further divided by considering the left-right context, that is, the bases adjacent to the point of substitution. The convention is to label the context in terms of the bases (C, T, A, or G) on the 5′ and 3′ sides on the strand containing the pre-substitution pyrimidine. For instance, in a substitution C>A, the C may be flanked by a T on the 5′ side and a G on the 3′ side:

TABLE 2

| | |
|---|---|
| before TCG | after TAG |

| | | | |
|---|---|---|---|
| 5′ | 3′ | 5′ | 3′ |

There are 4×4=16 different contexts for each of the original six substitution types. Therefore, there are 16×6=96 substitution types when context is taken into account.

At each position in the genome (except for a negligible fraction at chromosome ends), one of the two strands contains a pyrimidine C or T, flanked by bases on the 5′ and 3′ sides, say, X and Y, respectively: i.e., XCY or XTY. This defines 2×4×4=32 possible states at each point. For each such state, a mutation causing a substitution at this point can result in one of three possibilities:

TABLE 3

| before | 1 | 2 | 3 |
|---|---|---|---|
| XCY | XAY | XGY | XTY |
| XTY | XAY | XCY | XGY |

Note that, as before, there are 32×3=96 types of substitution.

Focusing still on one position [symbol] in the genome, assume mutations at [symbol] occur as a time-homogeneous continuous-time Markov process, holding the left-right context fixed (i.e., holding the neighboring base pairs constant). More precisely, when the current state is a, it remains a for an Exp(|Λₐₐ|) amount of time and then transitions to b≠a with probability Λ_{ab}/|Λₐₐ|, where Λ is a 32×32 matrix such that (i) Λ_{ab}≥0 for a a≠b, and (ii) Σ_{b}Λ_{ab}=0. This is equivalent to saying that transitions from a to b occur with rate Λ_{ab}; thus, Λ is called the transition rate matrix.

Let [symbol] denote the state at locus [symbol] at time t, and let [symbol] be the state at [symbol] for the normal (germline) genome of the individual under consideration. Let P_{ab}^{t}=P(Sₗ^{t}=b|Sₜ⁰=a) the probability that the state is b at time t given that the state is a at time 0. From the theory of continuous-time Markov chains, we have that

$${P}^{t} = exp ( t Λ ) = {\sum }_{k = 0}^{∞} \frac{{( t Λ )}^{k}}{k !} \qquad Equation 13$$

where exp(⋅) denotes the matrix exponential. Since the mutation rates Λ_{ab} are very small, it is reasonable to use a first-order Taylor approximation, P^{t}≈I+tΛ.

Let aᵢ and bᵢ denote the starting and ending states, respectively, for each of the substitution types i=1, . . . , 96. Let λ₁=Λ_{aibi}, and define Xᵢ^{T}=#{l: Sₗ⁰=aᵢ, Sₗ^{t}=bᵢ}, i.e., Xᵢ^{t} is the number of positions that undergo substitution i.

Consider all of the positions [symbol] that are in state a at time 0, and to simplify the math, let us assume that no two of these positions are adjacent. Of the 32 states, only four of them can be reached from a: the state can remain at a, or one of three substitutions can occur. Suppose these three substitutions are i=1, 2, 3, so that the starting states are a₁=a₂=a₃=a and the ending states are b₁, b₂, b₃, respectively. Letting n=#{l: sₜ⁰=a}, x₀=x₁+x₂+x₃, and λ₀=λ₁+λ₂+λ₃, we have

$$ℙ ( {X}_{1 : 3}^{t} = {x}_{1 : 3} \vert {S}^{0} = {s}^{0} ) = \frac{n !}{( n - {x}_{0} ) ! {x}_{1} ! {x}_{2} ! {x}_{3} !} {( {P}_{aa}^{t} )}^{n - {x}_{0}} {\prod }_{i = 1}^{3} {( {P}_{{a}_{i} {b}_{i}}^{t} )}^{{x}_{i}} \approx \frac{n !}{( n - {x}_{0} ) ! {x}_{1} ! {x}_{2} ! {x}_{3} !} {( 1 + {t λ}_{0} )}^{n - {x}_{0}} {( {t λ}_{1} )}^{{x}_{1}} {( {t λ}_{2} )}^{{x}_{2}} {( {t λ}_{3} )}^{{x}_{3}} \qquad Equation 14$$

Since n is large and tλ₀ is on the order of 1/n, then letting c=ntλ₀ we have (1−tλ₀)ⁿ=(1−c/n)n≈e^{-c}=exp(−ntλ₀) and (1−tλ₀)^{-xi}=(1−c/n)^{-x0}≈1 when xₒ<<n, which is the case with high probability. Plugging these into Equation 14 yields

$$\approx \frac{n !}{( n - {x}_{0} ) ! {x}_{1} ! {x}_{2} ! {x}_{3} !} exp ( - {nt λ}_{0} ) {( {t λ}_{1} )}^{{x}_{1}} {( {t λ}_{2} )}^{{x}_{2}} {( {t λ}_{3} )}^{{x}_{3}} = \frac{n ! {n}^{- {x}_{0}}}{( n - {x}_{0} ) !} {\prod }_{i = 1}^{3} exp ( - {nt λ}_{i} ) \frac{{( {nt λ}_{i} )}^{{x}_{i}}}{{x}_{i} !} \qquad Equation 15$$

By Stirling's approximation,

$$\frac{n ! {n}^{- {x}_{0}}}{( n - {x}_{0} ) !} \sim \frac{\sqrt{2 π n} {( n / e )}^{n} {n}^{- {x}_{0}}}{\sqrt{2 π ( n - {x}_{0} )} {( ( n - {x}_{0} ) / e )}^{n - {x}_{0}}} = \sqrt{\frac{n}{n - {x}_{0}}} \frac{{e}^{- {x}_{0}} {n}^{n}}{{( n - {x}_{0} )}^{n}} \frac{{( n - {x}_{0} )}^{{x}_{0}}}{{n}^{{x}_{0}}} \to 1 \qquad Equation 16$$

as n→∞. Hence, we have

$$ℙ ( {X}_{1 : 3}^{t} = {x}_{1 : 3} \vert {S}^{0} = {s}^{0} ) \approx {\prod }_{i = 1}^{3} Poisson ( {x}_{i} \vert {n}_{i} {t λ}_{i} )$$

when n is large, x₀<<n, and tλ₀ is on the order of 1/n.

For each of the 32 states a, the same approximation applies to the set of positions starting in state a. Modeling these 32 sets independently, we have

$$ℙ ( {X}_{1 : 96}^{t} = {x}_{1 : 96} \vert {S}^{0} = {s}^{0} ) \approx {\prod }_{i = 1}^{96} Poisson ( {x}_{i} \vert {n}_{i} {t λ}_{i} )$$

where nᵢ=#{ [symbol] : sₜ⁰=aᵢ}. In other words, the counts of the 96 substitution types are approximately distributed as independent Poisson random variables with rates nᵢtλᵢ.

The preceding derivation ignores the fact that a substitution at one position changes the context of the two adjacent positions. However, since it is rare for mutations to occur at two adjacent positions, this should be negligible.

Suppose counts xᵢⱼ are obtained for subjects j=1, . . . , J, for substitution types i=1, . . . , I,

where I=96. The derivation above suggests using the following model:

Poisson(xᵢⱼ|nᵢⱼλᵢⱼtⱼ) Equation 19

where tⱼ is the age (or exposure time) of subject j, and nᵢⱼ is the number of positions that are in state aᵢ in the normal genome of subject j, out of all positions that were measured, which maybe a subset of the genome due to low sequencing depth or exome sequencing for example.

Each subject is exposed to multiple mutational processes, e.g., UV radiation, smoking, replication errors, etc., each of which has a different rate profile across the 96 substitution types. Since rates are additive in a continuous-time Markov process, it is natural to model the subject-specific mutation rates λᵢⱼ as linear combinations of these mutational process rate profiles, with nonnegative weights depending on the exposure of the subject to each process. Thus, we assume a representation of the form

$${λ}_{ij} {t}_{j} = {\sum }_{k = 1}^{K} {r}_{ik} {θ}_{jk} \qquad Equation 20$$

where the weight θⱼₖ>0 is the exposure of subject j to process k, and (r₁ₖ, . . . , r₁ₖ) is the rate profile for mutational process k.

The exposures θⱼₖ are measured in units of time, and the rates rⱼₖ are measured in mutations per time unit for a single position. If we measure time in years, then the parameters have the following interpretation:

nᵢⱼrᵢₖ is the expected number of substitutions of type i in subject j due to one year of exposure to process k.

θⱼₖ is the number of years that subject j has been exposed to process k.

Since the rates for single position are exceedingly small, it is convenient to put them on a more interpretable scale by moving a fixed constant from nᵢⱼ to rᵢₖ. Specifically, by moving a factor of 10⁶, one can measure nᵢⱼ in Mbp (millions of basepair positions) and measure rᵢⱼ in mutations/Mbp/year.

Due to sequencing errors and other technical artifacts, occasionally a position will be erroneously counted as mutated. To account for this in the model, we consider the k=1 process to represent technical errors, and we set the corresponding profile to a fixed vector of rates (r₁₁, . . . r_{I1}) based on historical data.

A statistical issue with this particular representation is that there is a non-identifiability between the rᵢₖ's and θⱼₖ's, since arbitrary constants cₖ can be moved between them. This makes it problematic to consistently estimate r and θ from x data alone. One way of dealing with this is to normalize the r's by enforcing the constraint Σᵢ₌₁^{j}rᵢₖ=1 for all k.

However, from a Bayesian perspective, this non-identifiability is not a fundamental problem since the posterior distribution is still well defined. Additional data on process-specific mutation rates (such as experimental data) can be used to help estimate rᵢₖ, resolving the non-identifiability. A practical disadvantage of enforcing the normalization constraint is that it complicates inference somewhat when the start counts nᵢⱼ vary across subjects j. We explore both normalized and unnormalized approaches.

Often, covariates such as age, sex, smoking habits, family history, UV exposure, and so on, are available on each subject. Later, we construct a regression-based prior on the θ's that takes into account subject-specific covariates. Further, we present an algorithm for inferring the regression coefficients.

First, we present a simplified model that is interesting in its own right, and illustrates some key aspects of the inference algorithm we propose. Later, we will augment the model by (i) introducing indicator variables to allow θⱼₖ values to be exactly 0 (enabling inference for K as well as inferring which processes are active in which subjects), (ii) incorporating the start counts nᵢⱼ, and (iii) using a regression model for the prior on θⱼₖ in order to handle covariates.

We describe two versions of the model: a normalized version with Σᵢrᵢₖ=1, and an unnormalized version. Suppose

Xᵢⱼ|r,θ˜Poisson(Σₖ₌₁^{K}rᵢₖθⱼₖ) independently for i=1, . . . ,I, j=1, . . . ,J, θⱼₖ˜Gamma(a,b) independently for j=1, . . . J, k=1, . . . ,K, (rᵢₖ, . . . ,r_{Ik})˜Dirichlet(α1, . . . ,αI) independently for k=1, . . . ,K, Equation 21

for the normalized version, or

rᵢₖ˜Gamma(α,β) independently for i=1, . . . ,I, k=1, . . . ,K, Equation 22

for the unnormalized version. The likelihood for this model is

$$p ( x \vert r , θ ) = {\prod }_{i = 1}^{I} {\prod }_{j = 1}^{J} p ( {x}_{ij} \vert r , θ ) = {\prod }_{i , j} exp ( - {\sum }_{k} {r}_{ik} {θ}_{jk} ) \frac{{( {\sum }_{k} {r}_{ik} {θ}_{jk} )}^{{x}_{ij}}}{{x}_{ij} !} \qquad Equation 23$$

Due to the sum over k in the factor (Σₖrᵢₖθⱼₖ)^{x}^{ᵢⱼ}, it seems that there do not exist conditionally conjugate priors for the r's and θ's. Interestingly, however, by introducing a well-chosen auxiliary variable, we can find conditionally conjugate priors.

Let us introduce

Yᵢⱼ=(Yᵢⱼ₁, . . . ,Yᵢⱼₖ)|x,r,θ˜Multinomial(xᵢⱼ,(qᵢⱼ₁, . . . ,q_{ijK})) Equation 24

for i=1, . . . , I and j=1, . . . , J, where qᵢⱼₖ=rᵢₖθⱼₖ/Σₖ₌₁^{K}rᵢₖθⱼₖ. Then

$$p ( {y}_{ij} \vert x , r , θ ) = \frac{{x}_{ij} !}{{\prod }_{k} {y}_{ijk} !} {\prod }_{k = 1}^{K} {q}_{ijk}^{{y}_{ijk}} = \frac{{x}_{ij} !}{{( {\sum }_{k} {r}_{ik} {θ}_{jk} )}^{{x}_{ij}}} {\prod }_{k = 1}^{K} \frac{{( {r}_{ik} {θ}_{jk} )}^{yijk}}{{y}_{ijk} !} \qquad Equation 25$$

Since Σₖyᵢⱼₖ for all (yᵢₖ₁, . . . , y_{ijK}) in the support of this distribution. Therefore,

$$p ( x \vert r , θ ) p ( y \vert x , r , θ ) = {\prod }_{i , j} p ( {x}_{ij} \vert r , θ ) p ( {y}_{ij} \vert x , r , θ ) = {\prod }_{i , j , k} exp ( - {r}_{ik} {θ}_{jk} ) \frac{{( {r}_{ik} {θ}_{jk} )}^{{y}_{ijk}}}{{y}_{ijk} !} , \qquad Equation 26$$

which is, incidentally, equal to Π_{i,j,k} Poisson(yᵢⱼₖ|rᵢₖθⱼₖ). Thus, the auxiliary variable Y has the effect of breaking up the sum over k, and as a result it is easy to do inference using Gibbs sampling on the augmented joint distribution.

The augmented joint distribution is p(x, y, r, θ)=p(x|r, θ)p(y|x, r, θ)p(r)p(θ). The full conditionals are derived as follows.

Updating y. Sample from the multinomial distribution in Equation 24.

Updating θ. For each j and k,

$$p ( {θ}_{jk} \vert ⋯ ) ∝ p ( x \vert r , θ ) p ( y \vert x , r , θ ) p ( {θ}_{jk} ) ∝ ( {\prod }_{i} {{e}^{- {r}_{ik} {θ}_{jk}} ( {r}_{ik} {θ}_{jk} )}^{{y}_{ijk}} ) {θ}_{jk}^{a - 1} {e}^{- b {θ}_{jk}} ∝ {θ}_{jk}^{A - 1} {e}^{- B {θ}_{jk}} ∝ Gamma ( {θ}_{jk} \vert A , B ) \qquad Equation 27$$

where A=a+Σᵢ₌₁^{I}yᵢⱼₖ and B=b+Σᵢ₌₁^{I}rᵢₖ.

Updating r (normalized case).

$$p ( {r}_{Ik} , … , {r}_{Ik} \vert ⋯ ) ∝ p ( x \vert r , θ ) p ( y \vert x , r , θ ) p ( {r}_{Ik} , … , {r}_{Ik} ) ∝ ( {\prod }_{i , j} {{e}^{- {r}_{ik} {θ}_{jk}} ( {r}_{ik} {θ}_{jk} )}^{{y}_{ijk}} ) {\prod }_{i} {r}_{ik}^{{a}_{i} - 1} {∝}^{( a )} ( {\prod }_{i , j} {r}^{{y}_{ijk}^{ik}} ) {\prod }_{i} {r}_{ik}^{{a}_{k} - 1} = {\prod }_{i} {r}_{ik}^{{A}_{i} - 1} ∝ Dirichlet ( {r}_{Ik} , … , {r}_{Ik} \vert {A}_{1} , … , {A}_{l} ) \qquad Equation 28$$

where Aᵢ=αᵢ+Σⱼ₌₁^{J} [symbol] , and step (a) holds since Σᵢrᵢₖ=1 and thus Σᵢe^{-r}^{ᵢₖ}^{θ}^{ⱼₖ}=e^{-θ}^{ⱼₖ}Σᵢ^{r}^{ᵢₖ}=e^{-θ}^{ⱼₖ}, which does not depend on r.

Updating r (unnormalized case).

$$p ( {r}_{ik} \vert ⋯ ) ∝ p ( x \vert r , θ ) p ( y \vert x , r , θ ) p ( {r}_{Ik} ) ∝ ( {\prod }_{j} {{e}^{- {r}_{ik} {θ}_{jk}} ( {r}_{ik} {θ}_{jk} )}^{{y}_{ijk}} ) {r}_{ik}^{α - 1} {e}^{- β {r}_{ik}} ∝ {r}_{ik}^{A - 1} {e}^{- {Br}_{ik}} ∝ Gamma ( {r}_{ik} \vert A , B ) \qquad Equation 29$$

where A=α+Σⱼ₌₁^{J} [symbol] and B=β+Σⱼ₌₁^{J}θⱼₖ.

To accurately recover the true values of r and θ, it is essential to infer K (the number of mutational processes). A principled Bayesian approach would be to place a prior on K and simply consider the posterior distribution on K. However, this tends to be complicated and computationally burdensome, involving dimension-changing moves or using indicator variables to include or exclude a subset of processes.

Automatic relevance determination (ARD) is an expedient alternative that effectively selects a subset of processes by driving the coefficients of any unneeded processes to be close to zero. For some models, ARD can be implemented by maximization of the marginal likelihood (also known as type-II maximum likelihood). However, for the NMF models we are interested in, the marginal likelihood does not have a closed form. Nonetheless, ARD can still be performed via a particular choice of data-dependent hyperprior that we describe here. The basic idea is to choose a hyperprior that favors having few active processes, and the key is to calibrate the strength of the hyperprior to match the corresponding likelihood, so that it is not too strong and not too weak.

First, as usual in ARD, we parameterize the model in a way that gives each process k a common scale. In the r-normalized version of the model, we modify the prior on θ:

θⱼₖ˜Gamma(α,α/μₖ) Equation 30

and in the unnormalized version, we also modify the prior on r:

θⱼₖ˜Gamma(α,α/μₖ)

rᵢₖ˜Gamma(α,α/μₖ), Equation 31

with a common mean μₖ= [symbol] (θⱼₖ|μₖ)= [symbol] (rᵢₖ|μₖ) for all parameters involved in process k. Then, in both versions we use a hyperprior on the means: μₖ˜InverseGamma(a₀, b₀) for k=1, . . . , K. The full conditional distribution for μₖ is then, for the normalized version,

$$p ( {μ}_{k} \vert ⋯ ) ∝ ( {\prod }_{j} p ( {θ}_{jk} \vert {p}_{k} ) ) p ( {μ}_{k} ) ∝ ( {\prod }_{j} \text{?} \text{?} ) \text{?} \text{?} ∝ Inverse Gamma ( {μ}_{k} \vert A , B ) \\ \text{?} \text{indicates text missing or illegible when filed} \qquad Equation 32$$

where A=a₀+Jₐ and B=b₀+aΣᵢθⱼₖ, and for the unnormalized version,

$$p ( {μ}_{k} \vert ⋯ ) ∝ ( {\prod }_{i} p ( {r}_{ik} \vert {μ}_{k} ) ) ( {\prod }_{j} p ( {θ}_{jk} \vert {μ}_{k} ) ) p ( \text{?} ) ∝ ( \text{?} {μ}_{k}^{- α} \text{?} ) ( {\prod }_{j} {μ}_{k}^{- α} \text{?} ) \text{?} \text{?} ∝ Inverse Gamma ( {μ}_{k} \vert A , B ) \\ \text{?} \text{indicates text missing or illegible when filed} \qquad Equation 33$$

where A=a₀+I_{α}+Ja and B=b₀+aΣᵢrᵢₖ+aΣᵢθⱼₖ. The full conditionals for θⱼₖ and rᵢₖ are modified to use α/μₖ or α/μₖ instead of b or β.

The key is to choose a₀ and b₀ appropriately, so as (i) to favor small values of μk and (ii) to have appropriately calibrated strength so that the hyperprior will be just strong enough to drive down μk for any unneeded processes. To see how to do this, we will focus on the normalized version; the unnormalized version is similar. Since the prior mean is [symbol] (μₖ)=b₀/(a₀−1), in order to accomplish b₀=ε(a₀−1), we can choose for some relatively small ε, so that [symbol] (μₖ)−ε. For (ii), note that the full conditional for μk has mean

$$\frac{B}{A - 1} = \frac{{b}_{0} + a {\sum }_{j} {θ}_{jk}}{{a}_{0} + Ja - 1} = \frac{{a}_{0} - 1}{{a}_{0} - 1 + Ja} ( \frac{{b}_{0}}{{a}_{0} - 1} ) + \frac{Ja}{{a}_{0} - 1 + Ja} ( \frac{1}{J} {\sum }_{j = 1}^{J} {θ}_{jk} ) \qquad Equation 34$$

a convex combination of the prior mean b₀/(a₀−1) and the average

$$\frac{1}{J} {\sum }_{j = 1}^{J} {θ}_{jk} .$$

Thus, in order to make the prior have commensurate strength, we need to choose to be the same order of magnitude as Jₐ. Empirically, just choosing a₀=Jₐ+1 seems to work fine.

For processes k that are unneeded to fit the data, the parameters μk will be driven to θ, or more precisely, driven to ≈ε. Consequently, ε should be chosen to be significantly smaller than the values of θⱼₖ that one expects to encounter.

Here, we put together the pieces from the previous sections, to provide the Markov chain Monte Carlo (MCMC) algorithm for this basic model.

Input: x∈ [symbol] ₀^{J×J}, K, ε, a, and α

Output: Samples of θ∈ [symbol] _{>0}^{J×K}, r∈ [symbol] _{>0}^{t×K}, and μ∈ [symbol] _{<0}^{K}.

Set a₀=Jₐ+1 (normalized) or a₀=Ia+Ja+1 (unnormalized), and set b₀=ε(n₀−1)|

Initialize θ, r, and μ randomly (e.g., by sampling from the prior).

Iteratively repeat the following steps to obtain MCMC samples:

1. Sample [symbol] ˜Multinomial(xᵢⱼ, (qᵢⱼ₁, . . . , q_{ijK})) where qᵢⱼₖ=rᵢₖθⱼₖ/Σₖ₌₁^{K} r_{ik′}θ_{jk′} for i=1, . . . , I and j=1, . . . , J.

2. Sample θⱼₖ˜Gamma(A,B) where A=a+Σᵢ₌₁^{J}yᵢⱼₕ and B=a/μₖ+Σᵢ₌₁^{J}rᵢₖ for j=1, . . . , J and k=1, . . . , K.

3. (Normalized version) Sample (rᵢₖ, . . . , rₗₖ)˜Dirichlet(A₁, . . . , Aₜ), where Aₜ=a+Σⱼ₌₁^{J}yᵢⱼₖ, for k=1, . . . , K.

(Unnormalized version) Sample rᵢₖ˜Gamma(A, B) where A=a+Σⱼ₌₁^{J}yᵢⱼₖ and for B=a/μₖ+Σⱼ₌₁^{J}θⱼₖ, i=1, . . . , I and k=1, . . . , K.

4. (Normalized version) Sample μₖ˜InverseGamma(A, B) where A=a₀+Jₐ and B=b₀+aΣⱼ₌₁^{J} θⱼₖ, for k=1, . . . , K.

(Unnormalized version) Sample μₖ˜InverseGamma(A, B) where A=a₀+Iₐ+Jₐ and B=b₀+aΣⱼ₌₁^{J} θⱼₖ, for k=1, . . . , K.

To briefly demonstrate this model, we apply it to simulated data. Data for three different scenarios are generated: (a) (I, J, K₀)=(96,100,5), (b) (I, J, K₀)=(96,200,10), and (c) (I, J, K₀)=(96,400,20). For each scenario, we generate 10 simulated data sets by sampling true parameters θⱼₖ⁰˜Gamma(1, 0.5) and rᵢₖ⁰˜Gamma(0.5, 0.5) independently, and then sampling data xᵢⱼ˜Poisson(Σₖ₌₁^{Ka}rᵢₖ⁰θⱼₖ⁰) for i=1, . . . , I and j=1, . . . , J independently given the parameters.

For the model, we choose a=1, a=0.5, and set K to be (a) 10, (b) 20, and (c) 30 for

the three scenarios, respectively. We use ε=0.01 for the ARD tolerance parameter. For illustration purposes, we only use the normalized version of the model; the unnormalized version yields similar results. On each data set, we run the MCMC chain for 2000 iterations total, discarding the first 1000 as burn-in.

The model is symmetric up to permutations of the labels k, so label switching is a potential issue complicating estimation of the parameters. However, empirically we do not observe any label switching, so averaging across MCMC samples provides interpretable estimates of the parameters, even though in theory one would need to account for the possibility of label switching.

To estimate K₀, we apply a threshold to the estimated hyperparameters \hat{μ}k, keeping only those processes k such that \hat{μ}k>4ε.

To facilitate comparison with the true parameters (which are not identifiable due to the possibility of moving a constant cₖ between rᵢₖ⁰ and θⱼₖ⁰ we normalize the true parameters by setting θⱼₖ^{N}=cₖθⱼₖ^{θ} and rᵢₖ^{N}=rᵢₖ^{0̅}/cₖ where c̅ₖ̅=Σᵢr_{μk}^{-θ}.

As a benchmark, we compare results with the ARD NMF algorithm of Tan and Fevotte (2013) (specifically, their [symbol] ¹ algorithm for Poisson data), with their parameters set to a=5 and their suggested choice of b. We use the same values of K as for our algorithm. The Tan-Févotte algorithm seeks a maximum a posteriori (MAP) estimate. ARD NMF results for scenario (a) are given in Table 4.

TABLE 4

| | χ²(\hat{r}, | χ²(r^{N}, | rmse(\hat{r}, | rmse(\hat{θ}, | time |
|---|---|---|---|---|---|
| | r^{N}) | \hat{r}) | r^{N}) | θ^{N}) | (sec) |
| Our algorithm | 15.85 | 0.849 | 0.00155 | 25.27 | 50.26 |
| Tan-Fevotte | 984.98 | ∞ | 0.00186 | 35.77 | 2.55 |

ARD NMF results for scenario (b) are given in Table 5.

TABLE 5

| | χ²(\hat{r}, | χ²(r^{N}, | rmse(\hat{r}, | rmse(\hat{θ}, | time |
|---|---|---|---|---|---|
| | r^{N}) | \hat{r}) | r^{N}) | θ^{N}) | (sec) |
| Our algorithm | 961.3 | 7.01 | 0.00191 | 40.7 | 162.9 |
| Tan-Fevotte | 25809.7 | ∞ | 0.00303 | 120.0 | 16.08 |

ARD NMF results for scenario (c) are given in Table 6

TABLE 6

| | χ²(\hat{r}, | χ²(r^{N}, | rmse(\hat{r}, | rmse(\hat{θ}, | time |
|---|---|---|---|---|---|
| | r^{N}) | \hat{r}) | r^{N}) | θ^{N}) | (sec) |
| Our algorithm | 96.89 | 6.17 | 0.00471 | 101.5 | 503.6 |
| Tan-Fevotte | 18353.1 | ∞ | 0.00745 | 225.1 | 35.26 |

The results shown are averaged over the 10 randomly generated data sets for each scenario. In scenarios (a) and (b), both algorithms estimated the number of processes correctly in all 10 data sets, i.e., (a) \hat{K}=K₀=5 and (b) \hat{K}=K₀=10. In scenario (c), the estimates in the 10 runs were \hat{K}=20, 19, 21, 20, 20, 20, 19, 20, 19, 19 for our algorithm, and \hat{K}=18, 19, 19, 20, 19, 19, 18, 19, 18, 18 for the Tan-Févotte algorithm.

The distance metrics are defined as follows: χ²(p, q) is the chi-squared distance averaged across the columns, i.e.,

$${X}^{2} ( p , q ) = \frac{1}{K} {\sum }_{k = 1}^{K} {\sum }_{i = 1}^{I} {( {p}_{ik} - {q}_{ik} )}^{2} / {q}_{ik} .$$

Similarly, rmse(x, y) is the root-mean-squared error averaged across the columns, i.e.,

$$rmse ( xy ) = \frac{1}{K} {\sum }_{k = 1}^{K} {( \frac{1}{J} {\sum }_{j = 1}^{J} {( {x}_{jk} - {y}_{jk} )}^{2} )}^{1 / 2} .$$

Since the order of the columns is not identifiable, to compute the distances we first permute the columns to minimize the distance, using a greedy algorithm to avoid searching over all K! permutations. In cases where \hat{K}≠K₀, we pad the smaller matrix with dummy columns filled with 1/I for r and 0 for θ.

Our MCMC algorithm is consistently much more accurate in estimating r and θ than the Tan-Févotte MAP algorithm. For estimating K, the two methods seem to be comparable in accuracy. The reason why the Tan-Févotte estimates perform so poorly with respect to x²(r^{N}, \hat{r}) is that a number of its \hat{r}_{μk} values are very small or equal to 0, causing the chi-squared distance to blow up.

A further advantage of our MCMC algorithm is that it provides uncertainty quantification rather than simply point estimates. For example, the MCMC samples can be used to construct posterior credible intervals for quantities of interest.

A copy ratio is the relative concentration of copies of a given locus in a given sample. In other words, it is the proportion of copies of a given locus out of all copies of observed loci in a sample, divided by the corresponding proportion in a normal sample. Total copy ratio refers to the copy ratio including copies from both homologous chromosomes, as opposed to haplotype-specific copy ratio.

In order to estimate copy ratios from sequencing data, it is necessary to account for the fact that different loci have different propensities of being observed, due to the physics of the measurement technology. Further, a significant reduction in copy ratio estimation error can be obtained by measuring typical noise profiles from a large panel of normal samples, and subtracting off the projection onto this set of noise profiles.

Let nₛₗ:=number of reads of locus [symbol] in sample s. This is the observed data. Later, in the point mutation model, we will also consider the number of reference versus alternate reads. (Note: We use the notation a:=b to mean that a is defined to be b.)

Let Cₛ:=number of cells in sample s, for s=1, . . . , S. (Unobserved)

Let pₛₖ:=proportion of cells in samples from population k, for s=1, . . . , S, k=1, . . . , K. (Unobserved)

Let qₖₘ:=number of copies of segment m in each cell from population k, for k=1, . . . , K, m=1, . . . , M. (Unobserved for cancer populations, 2 for normal diploid populations)

Let tₖᵢ:=1 if population i is an ancestor of population k, otherwise tₖᵢ:=0 (for k=1, . . . , K, i=1, . . . , K). (Unobserved) We take the convention that the set of ancestors of k includes k itself, so tₖₖ=1. Note that this defines a phylogenetic tree with each population as a node.

Let mₗ:=index of the segment containing locus [symbol] , for [symbol] =1, . . . , L (Unobserved)

Let us define the normal population to be indexed by k=1.

We would like to infer the matrices P=(pₛₖ), Q=(qₖₘ), and T=(tₖᵢ), and the segmentation assignments mₑ. Estimating the copy ratios is the first step toward doing that.

One approach is to construct a generative model, and perform posterior inference given the nₛₗ's. However, standard inference techniques such as MCMC and variational Bayes do not seem to work well on this problem, since the space of Q's and T's is large and is complicated by strong dependencies and constraints, making the space difficult to explore.

The alternative explored here is to incrementally infer what we can from what we know, chipping away at the problem until we arrive at sets of plausible values of P, Q, and T. The plan is then to use these inferred values to construct an importance sampling distribution that will yield fully Bayesian inferences.

Cₛpₛₖ=number of cells in sample s from population k.

Cₛpₛₖqₖₘ=number of copies of segment m from population k, in samples.

Then the true concentration of locus l in sample s is

$$\frac{{\sum }_{k = 1}^{K} {C}_{s} {p}_{sk} \text{?}}{\text{?} {\sum }_{k = 1}^{K} {C}_{s} {p}_{sk} \text{?}} = \frac{{\sum }_{k = 1}^{K} {p}_{sk} \text{?}}{\text{?} {\sum }_{k = 1}^{K} {p}_{sk} \text{?}} \\ \text{?} \text{indicates text missing or illegible when filed} \qquad Equation 35$$

Dividing this by the true concentration of locus l for a normal sample, namely 1/L, yields the true copy ratio,

$${r}_{s 𝓁} := \frac{{\sum }_{k = 1}^{K} {p}_{sk} \text{?}}{\frac{1}{L} {\sum }_{{𝓁}^{′} = 1}^{L} {\sum }_{k = 1}^{K} {p}_{sk} \text{?}} \\ \text{?} \text{indicates text missing or illegible when filed} \qquad Equation 36$$

However, we do not get to observe the true copy ratio directly, since only a fraction of copies are observed as reads. Note that, by definition,

$$\frac{1}{L} {\sum }_{𝓁 = 1}^{K} \text{?} = 1 , \\ \text{?} \text{indicates text missing or illegible when filed}$$

so we only need to estimate the [symbol] up to a multiplicative constant, say ψₛ, such that [symbol] =ψₛΣₖ₌₁^{K} [symbol] . In particular, we have

$${r}_{s 𝓁} {∝}_{𝓁} {\sum }_{k = 1}^{K} {p}_{sk} \text{?} \\ \text{?} \text{indicates text missing or illegible when filed} \qquad Equation 37$$

where [symbol] denotes proportionality with respect to [symbol] .

Let [symbol] :=proportion of copies of locus [symbol] that are read (i.e., observed) in sample s.

Then [symbol] =Σₖ₌₁^{K} [symbol] =number of copies of locus [symbol] read in sample s. This is our observed number of reads, from above.

[symbol] = [symbol] Σₖ₌₁^{K} [symbol] =total number of reads in sample s, assuming each read contains only one locus.

So, from our observed data, we can compute the empirical copy ratio,

$${\hat{r}}_{s 𝓁}^{( 1 )} := \frac{{n}_{s 𝓁}}{\frac{1}{L} {\sum }_{{𝓁}^{′} = 1}^{L} {n}_{s {𝓁}^{′}}} = \frac{{γ}_{s 𝓁} {\sum }_{k = 1}^{K} {p}_{sk} {q}_{{km}_{i}}}{\frac{1}{L} {\sum }_{{𝓁}^{′} = 1}^{L} {γ}_{s {𝓁}^{′}} {\sum }_{k = 1}^{K} {p}_{sk} {q}_{{km}_{i}}} {α }_{𝓁} {γ}_{s 𝓁} {\sum }_{k = 1}^{K} {p}_{sk} {q}_{{km}_{i}} \qquad Equation 38$$

Note that this eliminates Cₛ, one of our unknowns.

However, comparing this with the true copy ratio, we see that we are still left with the issue of [symbol] varying across different loci [symbol] . As a first step toward mitigating this, we could divide by the empirical copy ratio for a normal sample s′, in which everything but the γ's cancel,

$${\hat{r}}_{{s}^{′} 𝓁}^{( 1 )} := \frac{{n}_{{s}^{′} 𝓁}}{\frac{1}{L} {\sum }_{{𝓁}^{′} = 1}^{L} {n}_{{s}^{′} 𝓁}} = \frac{{γ}_{{s}^{′} 𝓁}}{\frac{1}{L} {\sum }_{{𝓁}^{′} = 1}^{L} {γ}_{{s}^{′} {𝓁}^{′}}} {α}_{𝓁} {γ}_{{s}^{′} 𝓁} . \qquad Equation 39$$

However, there is still the issue that [symbol] ≠ [symbol] due to randomness. This noise can be reduced somewhat by averaging across a panel of normal samples s′∈S_{N},

$$\frac{1}{\vert {S}_{N} \vert} {\sum }_{{s}^{′} ϵ {S}_{N}}^{ } {\hat{r}}_{{s}^{′} 𝓁}^{( 1 )} = \frac{1}{\vert {S}_{N} \vert} {\sum }_{{s}^{′} ϵ {S}_{N}}^{ } \frac{{γ}_{{s}^{′} 𝓁}}{\frac{1}{L} {\sum }_{{𝓁}^{′} = 1}^{L} {γ}_{{s}^{′} 𝓁}} \approx \frac{1}{\vert {S}_{N} \vert} {\sum }_{{s}^{′} ϵ {S}_{N}}^{ } {ψγ}_{{s}^{′} 𝓁} \approx ψ𝔼 ( {γ}_{{s}^{′} 𝓁} ) {α}_{𝓁} 𝔼 ( {γ}_{{s}^{′} 𝓁} ) \qquad Equation 40$$

Here, the first approximation is by applying the law of large numbers to the denominators:

$$\frac{1}{L} {\sum }_{{𝓁}^{′} = 1}^{L} {γ}_{{s}^{′} {𝓁}^{′}} \to 1 / ψ as L \to ∞ ,$$

where 1/ψ is defined to be this limit, and it is assumed that [symbol] ˜ [symbol] independently for some distributions [symbol] . If L>>|S_{N}| then this is approximation is reasonable. The second approximation is by applying the law of large numbers as |S_{N}|→∞.

Thus, we could estimate rₛₗ by

$${\hat{r}}_{s 𝓁}^{( 2 )} := \frac{{\hat{r}}_{s 𝓁}^{( 1 )}}{\frac{1}{\vert {S}_{N} \vert} {\sum }_{{s}^{′} {ϵS}_{N}} {\hat{r}}_{{s}^{′} 𝓁}^{( 1 )}} \frac{{γ}_{s 𝓁}}{𝔼 ( {γ}_{s 𝓁} )} {\sum }_{k = 1}^{K} {p}_{sk} {q}_{{km}_{e}} , \qquad Equation 41$$

where [symbol] means approximately proportional to, as a function of [symbol] .

Still, there is quite a bit of noise in [symbol] / [symbol] . One more step is used to remove some of this noise. Note that if s′ is a normal sample, then [symbol] ≈ [symbol] / [symbol] .

Thus, the vectors represent \hat{r}ₛ⁽²⁾:=(\hat{r}_{s′1}⁽²⁾, . . . , r_{s′L}⁽²⁾)^{T}∈ [symbol] ^{L}, for s′∈S_{N}, represent directions (or, noise profiles) in L-dimensional space that are typical for samples of the random vector (γₛ₁/ [symbol] (yₛ₁), . . . , y_{sL}/ [symbol] (y_{sL}))^{T}∈ [symbol] ^{L}.

The idea is to remove any component of our cancer copy ratio profile estimate \hat{r}ₛ⁽²⁾=(\hat{r}ₛ₁⁽²⁾, . . . , \hat{r}_{sL}⁽²⁾)^{T} that can be explained by the noise profiles. Specifically, what is done is to center the data at zero (subtract off the mean), take the projection of our centered estimate onto the space spanned by the noise profiles, and then subtract this projection from the centered estimate. Another way of viewing this is that we are computing the residuals after regressing the estimate onto the noise profiles.

The mathematical details are as follows. For notational convenience, let's reindex the samples so that the normal samples are numbered s′=1, . . . , |S_{N}| (and the cancer sample s is not one of these). Let [symbol] = [symbol] −1 (i.e., subtract off the sample mean over s′), and form the matrix A=(α_{ts′})∈ [symbol] ^{L×|S}^{_{N}}^{|}.

Likewise, let [symbol] = [symbol] −1, where s is our cancer sample, and denote [symbol] =( [symbol] . . . , [symbol] )^{T}.

Let A⁺:=(A^{T}A)⁻¹A^{T} (i.e., A⁺ is the pseudoinverse of A). Then AA+ y is the projection of y onto the column space of A (i.e., onto the span of the mean-centered noise profiles). The residuals, then, are

\hat{r}ₛ⁽³⁾:= [symbol] −AA⁺ [symbol] =(I−AA⁺) [symbol] Equation 42

The vector \hat{r}ₛ⁽³⁾=(\hat{r}ₛ₁⁽³⁾, . . . , \hat{r}_{sL}⁽³⁾)^{T} is an improved estimate of total copy ratios for cancer sample s. This is repeated for each cancer sample s, using the same matrix A from the panel of normals.

The total copy ratio estimates [symbol] are then used (along with the alt/ref read counts at heterozygous loci) to obtain haplotype-specific copy ratio estimates. The haplotype-specific copy ratio estimates are, in turn, used to choose segment assignments [symbol] . This allelic CapSeg method is described further above.

From the segment assignments [symbol] and the locus-level total copy ratio estimates [symbol] , we can improve our estimates of total copy ratio by combining the estimates within each segment, since the true total copy ratio is constant within each segment (assuming the segmentation is valid). Thus, we can estimate total copy ratio for all loci in segment m as

$${\hat{r}}_{sm} α \frac{1}{\vert {L}_{m} \vert} {\sum }_{𝓁ϵ {L}_{m}} {\hat{r}}_{s 𝓁}^{( 3 )} \qquad Equation 43$$

where Lₘ:={ [symbol] : [symbol] =m} is the set of loci in segment m, and the constant of proportionality is determined by the constraint that

$$\frac{1}{L} {\sum }_{𝓁 = 1}^{L} {\hat{r}}_{{sm}_{𝓁}} = 1.$$

The standard error of this average (adjusted for multiplying the constant of proportionality), say \hat{σ}ₛₘ, can be used as a rough estimate of the standard deviation of the estimator \hat{r}ₛₘ.

Using a probabilistic model, or at least some form of shrinkage, can provide substantial improvement in copy ratio estimation accuracy. In high-dimensional estimation problems like this, empirical averages are often not very accurate (e.g., the classic example is Stein's paradox). A natural choice would be a Poisson model with sample-specific effects and locus-specific effects, along with some form of noise reduction using the normal panel.

Also, subtracting off the noise projection doesn't make complete sense to me. Since the noise [symbol] / [symbol] is multiplicative rather than additive, it seems like it would make more sense to divide by something rather than subtract something.

In order to separate the populations, as a next step toward recovering P, Q, and T, we will consider the estimated total copy ratios \hat{r}ₛₘ as our input data, along with estimates of the standard deviation \hat{σ}ₛₘ of the \hat{r}ₛₘ estimators.

Indexed by segment m, the true total copy ratios are rₛₘ:=ψₛΣₖ₌₁^{K}pₛₖqₖₘ, where ψₛ is such that

$$\frac{1}{L} {\sum }_{𝓁 = 1}^{L} {r}_{{sm}_{𝓁}} = 1.$$

Thus, defining ∈ₛₘ:=\hat{r}ₛₘ−rₛₘ, we have

$${\hat{r}}_{sm} = {ψ}_{s} {\sum }_{k = 1}^{K} {p}_{sk} {q}_{km} + {ɛ}_{sm} \qquad Equation 44$$

where the error εₛₘ should be on the order of \hat{σ}ₛₘ. Equivalently, in matrix notation,

R=ΨPQ+εᵢ Equation 45

letting \hat{R}:=(\hat{r}ₛₘ) and Ψ:=diag(ψ).

Let d₁ₘ:=2, and for k=2, . . . , K, let dₖₘ:=qₖₘ−qₚₐ_{ₖ}ₘ where paₖ is the parent of population k in the tree T. (k=1 is the normal population.) In other words, dₖₘ is the change in segment m's total copy number that occurred when population k arose from its parent population. We refer to dₖₘ as the copy delta at population k, for segment m.

Then qₖₘ=Σᵢ₌₁^{K} tₖᵢdᵢₘ. Therefore,

$${\sum }_{k = 1}^{K} {p}_{sk} {q}_{km} = {\sum }_{k = 1}^{K} {p}_{sk} {\sum }_{i = 1}^{K} {t}_{ki} {d}_{im} = {\sum }_{i = 1}^{K} {d}_{im} {\sum }_{k = 1}^{K} {p}_{sk} {t}_{ki} . \qquad Equation 46$$

Defining bₛᵢ=Σₖ₌₁^{K} pₛₖtₖᵢ, we therefore have

$${\sum }_{k = 1}^{K} {p}_{sk} {q}_{km} = {\sum }_{k = 1}^{K} {b}_{sk} {d}_{km} \qquad Equation 47$$

Equivalently, in matrix notation, Q=TD, where D=(dₖₘ). Therefore,

PQ=PTD=BD Equation 48

where B:=PT.

Plugging this into the equation for \hat{r}ₛₘ above, we get

$${\hat{r}}_{sm} = {\sum }_{k = 1}^{K} {ψ}_{s} {b}_{sk} {d}_{km} + {ɛ}_{sm} . \qquad Equation 49$$

Or, in matrix form, \hat{R}=ΨBD+ε.

The B matrix has a natural interpretation, namely, bₛₖ is the proportion of cells in sample s that are descended from population k (i.e., that belong to a population descended from k). We refer to the b's as descended cell fractions (DCFs).

For any given segment m, the copy deltas d₁ₘ, . . . , d_{Km} should be approximately independent across the different populations. This is not exactly true, since the copy number cannot be negative, and if it reaches zero then it cannot become positive after that. However, the idea is that d₁ₘ, . . . , d_{Km} are close enough to independent to be exploited to obtain a reasonable estimate in practice.

If d₁ₘ, . . . , d_{Km} are independent, and are not Gaussian distributed, then the problem of

recovering ΨB and D given \hat{R} is precisely the independent components analysis (ICA) problem.

Note that since bₛ₁=1 for all s, we can solve for Ψ and B given the product ΨB. Also, by definition, the first row of D is d₁=(2,2, . . . ,2,2), so we only need to infer the remaining rows. To simplify notation a bit, let's define A:=ΨB.

For this problem, ICA algorithms do not work well for recovering the full matrices A and D. In part, this is because the inverse transformations (the w's below) are often not orthogonal, as assumed in standard ICA, and in part it is because we have additional information about the distribution of the copy deltas dₖₘ.

Single-unit ICA may be applied to recover one row of D at a time. An estimate of D then directly yields an estimate of A via a pseudoinverse calculation.

First we look at how to estimate a single row of D using single-unit ICA. Then we show how to estimate A given an estimate of D. Finally, we describe how to find the remaining rows of D by using residual \hat{R} matrices.

The first step is to standardize the data matrix \hat{R} to have zero mean and covariance matrix equal to the identity, considering each column as an input vector. Specifically, let zₘ:=(\hat{r}₁ₘ, . . . , \hat{r}_{Sm})^{T} be the mth column of \hat{R}. Let C be the empirical covariance matrix of the z's, i.e.,

$$C \text{:} = \frac{1}{M} {\sum }_{m = 1}^{M} ( {z}_{m} - \overline{z} ) {( {z}_{m} - \overline{z} )}^{T} ,$$

where

$$\overline{z} = \frac{1}{M} {\sum }_{m = 1}^{M} {z}_{m} .$$

Factorize C as C=UVU^{T}, where the columns of U are the eigenvectors of C, and V is the diagonal matrix with the corresponding eigenvalues on the diagonal. Then, letting

xₘ:=UV^{-1/2}U^{T}(zₘ−z̅) Equation 50

we have that x₁, . . . , x_{M}∈ [symbol] ^{S} are linearly transformed versions of the columns of \hat{R} with zero empirical mean and empirical covariance equal to the identity.

After this standardization step, single-unit ICA searches for a vector w∈ [symbol] ^{S} with ∥w∥=1 such that the empirical distribution of w^{T}x₁, . . . , w^{T}x_{M}∈ [symbol] is as non-Gaussian as possible. This is done using a rapidly converging approximate Newton's method optimization initialized at a random w. Non-Gaussianity is measured by an approximation to negentropy.

A linear combination of independent variables is more Gaussian than each of the uncombined variables. As a result, Gaussianity should be minimized when w extracts one of the original rows of D (recall that d₁ₘ, . . . , d_{Km} are assumed to be roughly independent). More precisely, when Gaussianity is minimized, (w^{T}x₁, . . . , w^{T}x_{M}) should be approximately equal to one of the rows of D, up to a multiplicative factor of undetermined scale and sign.

To infer the scale a of the multiplicative factor, we use the fact that the entries of D are integers, mostly zeros or near zero. So in order for (θw^{T}x₁, . . . , θw^{T}x_{M}) to approximate a row of D, its entries should look like samples from a Gaussian mixture with components centered at the integers, with the mixture weights favoring the components near 0. Thus, we use the likelihood under such a Gaussian mixture model to infer the scale a and also to score the quality of w.

The single-unit ICA procedure does not always yield a high-quality w, since there are usually multiple local optima in the optimization. Thus, we generate a number of candidate w's by running the procedure with different randomly chosen initializations.

The candidate w's are then ranked by the mixture model-based score. The highest ranking w is selected, and an estimate of a row of D is formed by setting \hat{d}:=([σw^{T̅}x₁], . . . , [σw^{T}x_{M}]), where σ is the estimated scale factor and [y] denotes [symbol] ∈ [symbol] rounded off to the nearest integer. (This may be improved by assigning points to components using the mixture model.)

Given an estimate of the copy delta matrix \hat{D}, even if only for a subset of populations, we can obtain an estimate Â of the columns of ΨB for the corresponding populations.

This is done by first setting Â:=\hat{R}\hat{D}⁺, where \hat{D}⁺ is the pseudoinverse of \hat{D}.

Since the correct sign for each row of \hat{D} is not determined by ICA, we need to infer the appropriate sign for each column of Â. The entries of A must all be nonnegative. Thus, we can simply multiply each column of A by the sign of its average in order to correct the sign for that column. We then force all entries of Â to be nonnegative by setting any remaining negative values to 0. The same sign flips performed on columns of Â are also applied to the corresponding rows of \hat{D} to correct its signs as well.

To estimate the full matrices A and D, we use the procedures described above in a stepwise fashion, adding one population at a time, as follows.

Initialize with only the normal population: Â=(1, . . . 1)^{T}∈ [symbol] ^{S×1} and \hat{D}=(2, . . . , 2)∈ [symbol] ^{I×M}.

Then, repeat the following steps to add populations:

1. Compute the residual copy ratio matrix \hat{R}^{res}=R−Â\hat{D}

2. Apply the single-unit ICA procedure described above to \hat{R}^{res} (instead of \hat{R}), to obtain a new estimated row \hat{d}.

3. Append \hat{d} to \hat{D}, and update Â using the procedure above. Also correct the signs of \hat{D} as described there.

4. Stop adding populations if the newly added column of Â has very small entries and the score (or some other test statistic for fit) is not very good. Otherwise, go to step 1.

Discard any rows of \hat{D} that are low quality (using a criterion based on the score or some other test statistic for fit), and recompute Â as above.

Lastly, solve for the unique \hat{Ψ} and \hat{B} such that \hat{Ψ}\hat{B}=A, \hat{b}ₛ₁=1 for all s, and \hat{Ψ} is diagonal.

Occasionally it happens that at a given step, the newly added column of Â is nearly identical to an existing column. If this occurs (e.g., if the correlation is greater than 0.995), then discard this column and instead use the next highest scoring candidate \hat{d}.

With a minor adjustment, the same algorithm can be applied to haplotype-specific copy ratio (HSCR) data that is phased within each segment (but not necessarily phased across segments).

Let qₖₘ^{h} be the copy number of segment m of haplotype h, for a cell in population k (for h∈{1,2}). Here, it is assumed that within a given segment m, the label h refers to the same haplotype in all populations k, but importantly, the labeling does not have to be consistent from segment to segment.

Then rₛₘ^{h}:=ψₛΣₖ₌₁^{K} pₛₖqₖₘ^{h} is the haplotype-specific copy ratio of haplotype h for sample s and segment m. Here, ψₛ and p^{slc} are the same values as in the case of total copy ratio.

By definition, qₖₘ=qₖₘ¹+qₖₘ², and therefore, rₛₘ=rₛₘ¹+rₛₘ², the total copy ratio is the sum of the HSCRs.

An estimate \hat{r}ₛₘ^{h} of the HSCR rₛₘ^{h} can be obtained from an estimate \hat{r}ₛₘ of the total copy ratio along with alt/ref read counts at heterozygous (non-mutated) loci within the segment. Here, we will assume that estimates \hat{r}ₛₘ^{h} are given, which we will treat as input data, along with estimates \hat{σ}ₛₘ^{h} of the standard deviations of these estimators.

The main adjustment that needs to be made from the case of total copy ratio is that for the normal population k=1, the haplotype-specific copy numbers q₁ₘ^{h} are all equal to 1 rather than 2. So, to define the haplotype-specific copy deltas, we let d₁ₘ^{h}:=1 and for k=2, . . . , K, let dₖₘ^{h}:=qₖₘ^{h}−qₚₐ_{ₖ}ₘ^{h} where pâₖ is the parent of k in the tree T.

Then, just in the case of total copy ratio, in matrix form we have Qₕ=TD^{h} and thus R^{b}=ΨPQ^{h}=ΨBD^{h}. So, letting ∈^{h}=\hat{R}^{h}−R^{h}, we have

\hat{R}^{h}=ΨPQ^{h}+ε^{h}=ΨBD^{h}+ε^{h}. Equation 51

Therefore, in block matrix notation, letting \hat{R}^{HS}:=[\hat{R}¹ \hat{R}²], D^{HS}:=[D¹ D²], and ε^{HS}:=[ε¹ ε²]|

\hat{R}^{HS}=ΨBD^{HS}+ε^{HS} Equation 52

So, to obtain estimates \hat{B} and \hat{D}^{HS}, we can use the same algorithm as in the case of total copy ratio, but with \hat{R}^{HS} as input instead of \hat{R}, and with the first row of \hat{D}^{HS} set to 1 instead of 2.

Another adjustment is to modify the model distribution on haplotype-specific copy deltas to favor somewhat smaller values compared to the model on total copy deltas (since a total copy delta is the sum of the two haplotype-specific copy deltas).

The estimated haplotype-specific copy delta matrix \hat{D}^{HS} is phased across populations but not across segments (unless the input HSCR matrix \hat{R}^{HS} is phased across segments). In other words, \hat{d}ₖₘ¹ and \hat{d}_{k′m}¹ both refer to changes in the copy number of segment m for the same haplotype, however, \hat{d}ₖₘ¹ and \hat{d}_{km′}¹ may refer to changes in the copy number of segment m for different haplotypes.

The DCF matrix B provides a substantial amount of information about the tree T. In this section, we describe how to find the set of all trees that are consistent with an estimated DCF matrix \hat{B}, assuming estimates of the uncertainty in \hat{B} are also given. This is done using a recursive tree construction procedure combined with a sequence of hypothesis tests for the plausibility of parent-children relationships. A threshold for rejecting implausible trees can be adjusted to make the procedure more permissive (find more trees) or less permissive (find fewer trees).

Information about T can be recovered from B do to an inequality between a parent's DCF and the DCFs of its children.

B=PT, i.e., bₛₖ=Σᵢ₌₁^{K}pₛᵢtᵢₖ. Thus, bₛₖ is the proportion of cells in sample s that are descended from population k (i.e., that belong to a population descended from k).

Suppose the children of k are j₁, . . . , j_{N}. Then the sets of cells descended from j₁, . . . , j_{N} are disjoint, and all of these cells are descended from k. Thus,

bₛⱼ_{₁}+ . . . +bₛⱼ_{_{N}}≤bₛₖ Equation 53

for all s=1, . . . , S. In other words, the sum of the children is less or equal to their parent, in terms of DCFs. We refer to this as the child-sum inequality.

The child-sum inequality can also be derived as follows. tᵢₖ=1 if population i is a descendant of population k (including k itself), otherwise tᵢₖ=0. Thus, tᵢₖ= [symbol] (i=k)+tᵢⱼ_{₁}+ . . . +tᵢⱼ_{_{N}}, since (a) i is a descendant of k if either i=k or i is a descendant of a child of k, and (b) the descendants of the children do not overlap. (We use [symbol] for the indicator function, i.e., [symbol] (E) is 1 if E is true, and is 0 otherwise.) Hence, plugging this into the formula for bₛₖ, we have

$${b}_{sk} = {\sum }_{i = 1}^{K} ( ( i = k ) + {\sum }_{n} {t}_{{ij}_{n}} ) = {p}_{sk} + {\sum }_{n = 1}^{N} {\sum }_{i = 1}^{K} {p}_{si} {t}_{{ij}_{n}} = {p}_{sk} + {\sum }_{n = 1}^{N} {b}_{{sj}_{n}} . \qquad Equation 54$$

Note that in particular, the DCFs are non-increasing as we go down the tree, i.e., if i is a descendant of k, then bₛᵢ≤bₛₖ for all s.

It turns out that the child-sum inequality is a necessary and sufficient condition for a tree to be consistent with B, when P is unknown. Suppose B∈[0, 1]^{S×K} with bₛ₁=1 for all s, and T∈{0, 1}^{K×K} is a tree matrix with root 1. Then B and T satisfy the child-sum inequality for all s and k if and only if there exists a matrix P∈[0, 1]^{S×K} such that B=PT. B=PT implies the child-sum inequality. Suppose the child-sum inequality is satisfied for all s and k. For each s and k, define pₛₖ:=bₛₖ=Σₙ₌₁^{N} bₛⱼ_{ₙ} where j₁, . . . , j_{N} are the children of k.

Then pₛₖ≥0 for all s, k, and B=PT.

Here is described an algorithm that produces the set of trees that are consistent with B, when P is unknown. To convey the basic structure of the algorithm, we first consider the case in which B is known exactly.

By definition, we know that node 1 is the root of the true tree T, since it represents the normal population.

Given J⊇{1, . . . , K}, we say that a tree T′ with nodes J is valid if it has root 1 and all of the child-sum inequalities are satisfied with respect to the corresponding columns of B.

Given a rooted tree T′, an extension of T′ is a rooted tree that contains all of the nodes of T′ and exhibits the same ancestor-descendant relationships among these nodes as T′ (but not necessarily the same parent-child relationships).

The problem of finding all valid trees on {1, . . . , K} reduces to the following subproblem: given a tree T^{(k-1)} with nodes 1, . . . , k−1, find all valid extensions to 1, . . . , k (i.e., all valid ways of adding node k to the tree in a way that preserves the existing ancestor-descendant relationships).

This subproblem can be solved using the following recursive procedure, with input T^{(k-1)}. For each i=1, . . . , k−1, consider making k a child of i. Let Jᵢ be the children of i in T^{(k-1)}. For each subset J⊇Jᵢ, consider making J the children of k (and k a child of i), while preserving all other parent-child relationships.

1. If the child-sum inequality would be violated fork (with J being the children of k) or violated for i (with (Jᵢ/J)∪{k} being the children of i), then reject this extension.

2. Otherwise, accept the extension as T^{(k)}. If k=K then we have a complete valid tree, so add T^{(k)} to a running list of complete valid trees. If k<K then recurse to find all valid extensions of T^{(k)} to 1, . . . , k+1.

Several optimizations may be applied to this procedure:

If bₛₖ>bₛᵢ for some s, then we can reject all extensions in which k is a descendant of i. To use this efficiently, we can visit nodes i of T^{(k-1)} in a depth-first traversal, and if bₛₖ>bₛᵢ for some s, then skip to the first non-descendant of i in T^{(k-1)}.

The search over subsets J⊆Jᵢ can be sped up by using the fact that if J is not a valid set of children for k, then no superset J′⊇J is valid either.

This algorithm is guaranteed to generate all valid trees on {1, . . . , K}. Any tree with root 1 can be constructed by starting with 1 as root and incrementally adding nodes k=2, . . . , K by making k a child of some i∈{1, . . . , k−1} and moving a subset of the current children of this i to instead be children of k, while preserving all other parent-child relationships. If there is a valid extension of a tree, then that tree is valid. By construction, any tree generated by the algorithm is valid. Consider any valid tree T on 1, . . . , K. As above, T can be constructed via a sequence of node insertions as in the algorithm, and each of the intermediate trees T⁽¹⁾, . . . , T^{(K-1)} in this sequence is guaranteed to be valid (since T is a valid extension of each of them), so it will not be rejected. Therefore, the algorithm will generate T.

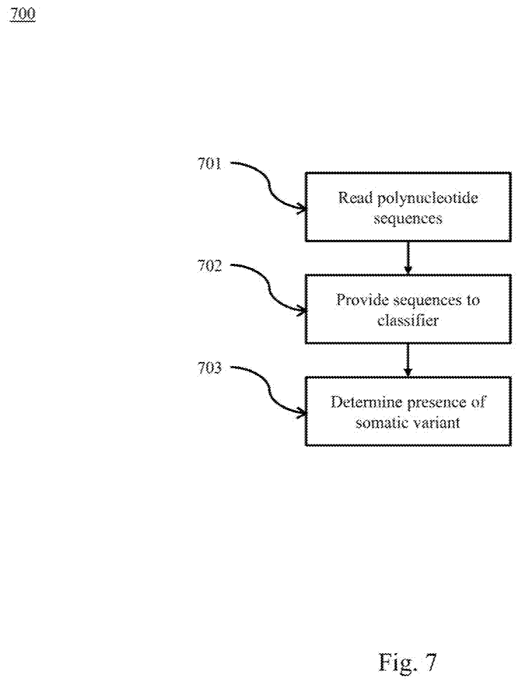

Referring now to FIG. 7, a method of analyzing a sample comprising a plurality of polynucleotides is illustrated. At 701, the plurality of polynucleotides is sequenced to obtain a plurality of sequences. At 702, the plurality of sequences is provided to a trained classifier. The trained classifier is adapted to accept a plurality of sequences and output a class label indicative of the presence of a somatic variant within the plurality of sequences. At 703, a class label is received from the trained classifier indicative of the presence of a somatic variant within the plurality of sequences.

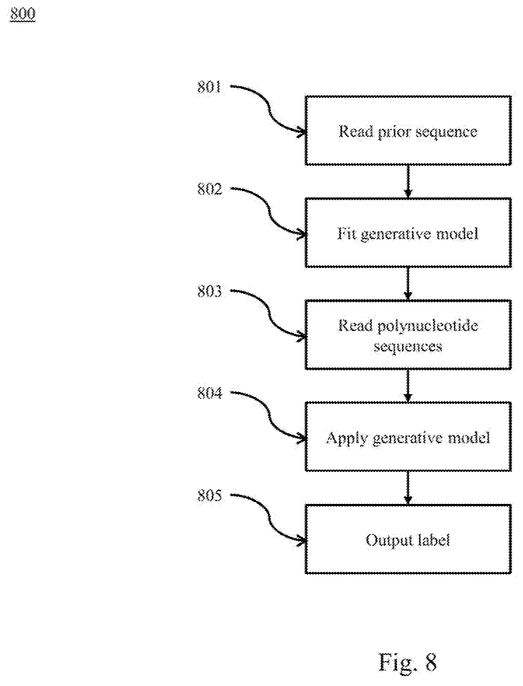

Referring now to FIG. 8, a method of analyzing a sample comprising a plurality of polynucleotides is illustrated. At 801, at least one prior sequence of a polynucleotide associated with a tumor genome is read. At 802, a generative model is fit to the at least one prior sequence. At 803, a plurality of sequences of the plurality of polynucleotides is read. At 804, the generative model is applied to the plurality of sequences to determine a probability that a first somatic clone is present in the plurality of sequences. At 805, based on the probability, a label indicative of the presence of the first somatic clone in the sample is determined.

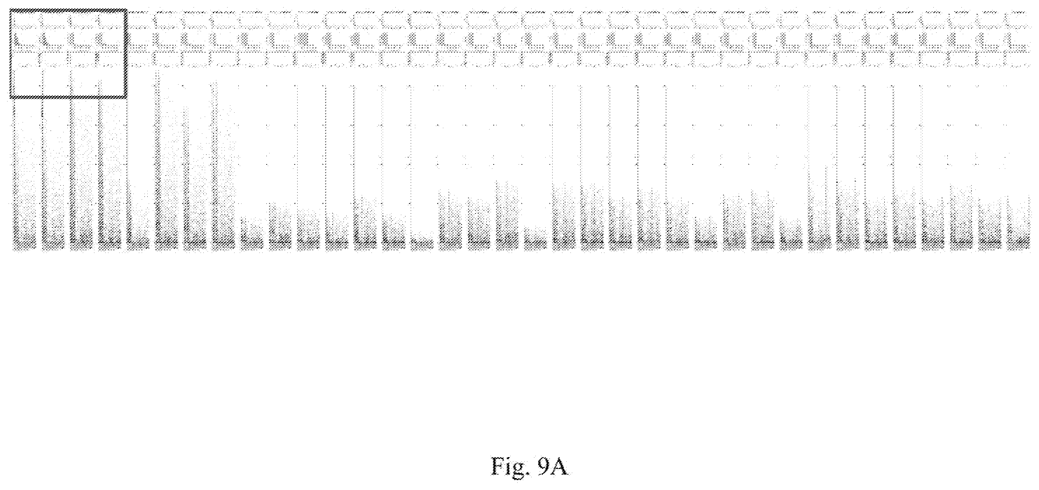

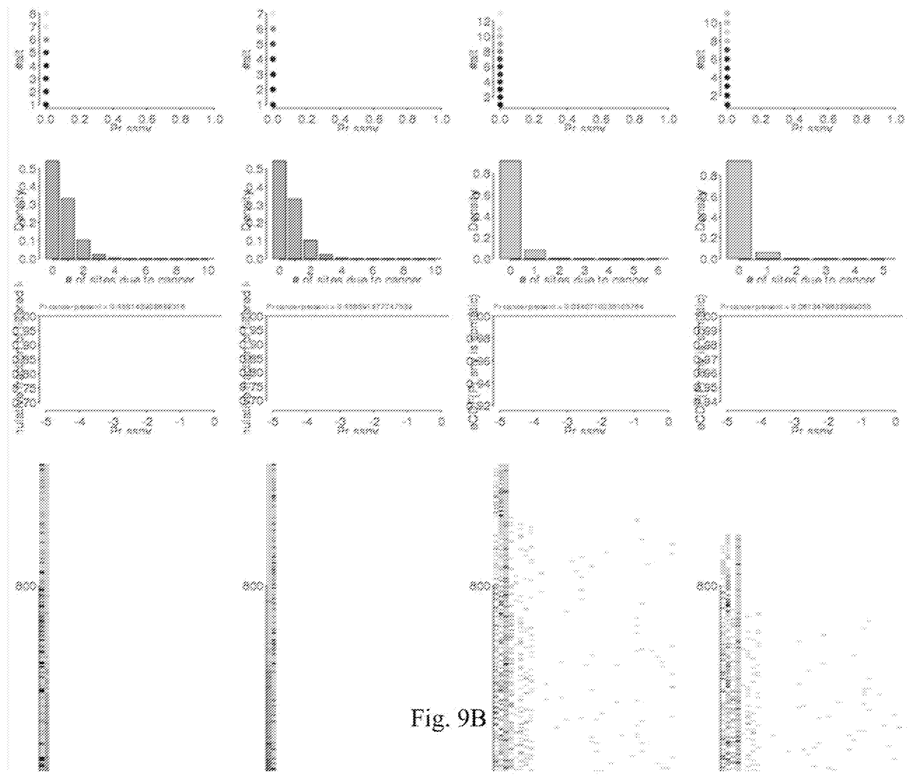

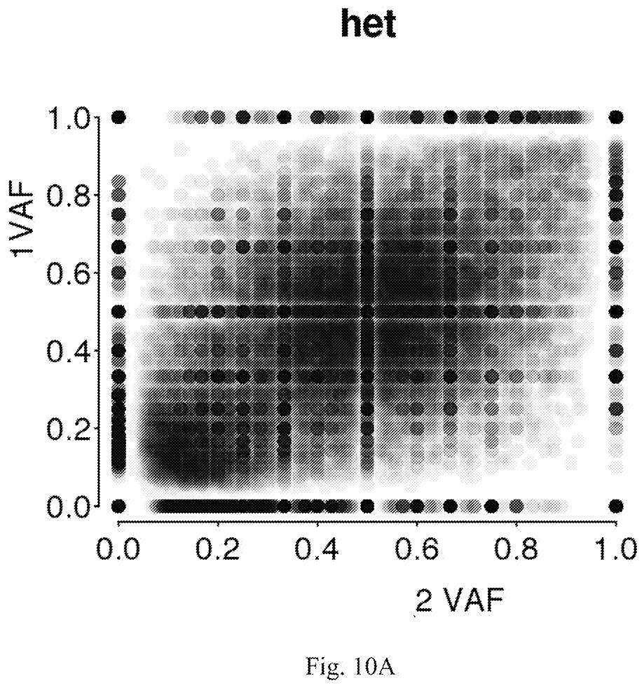

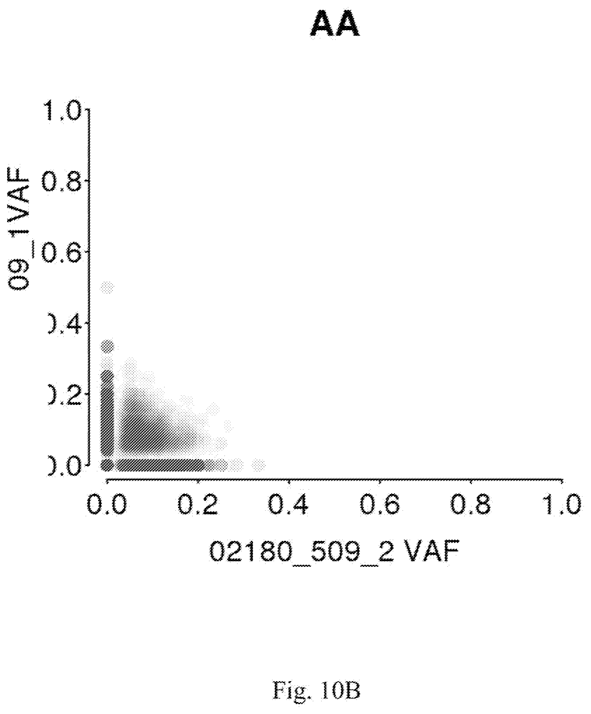

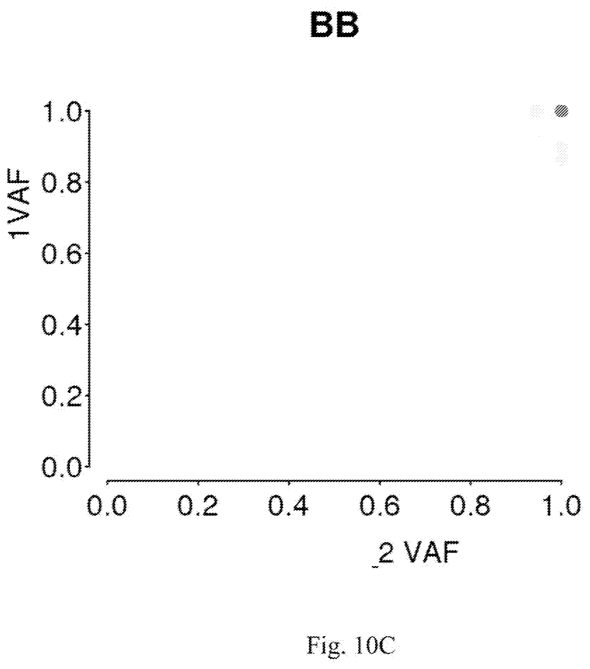

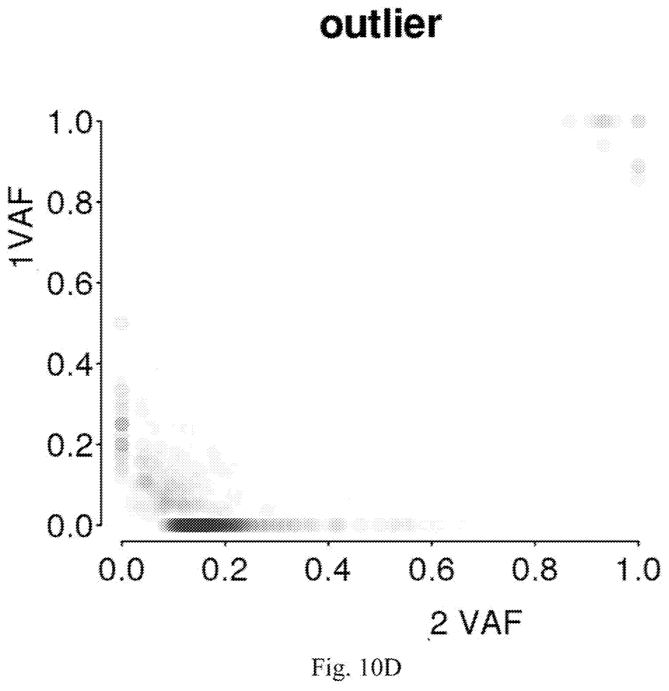

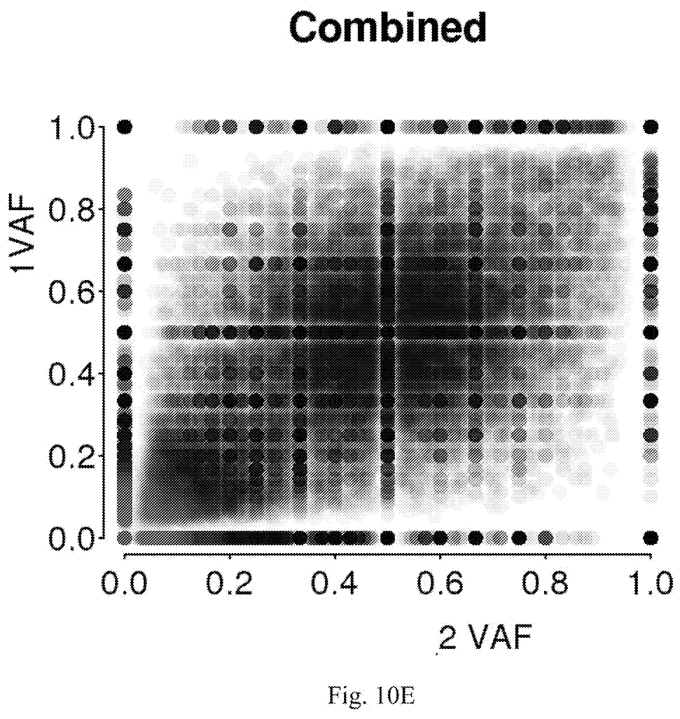

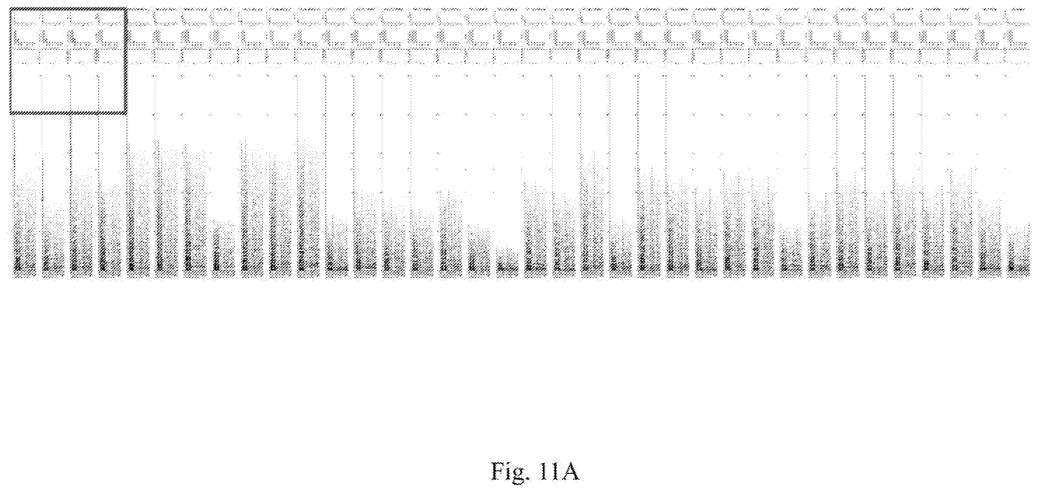

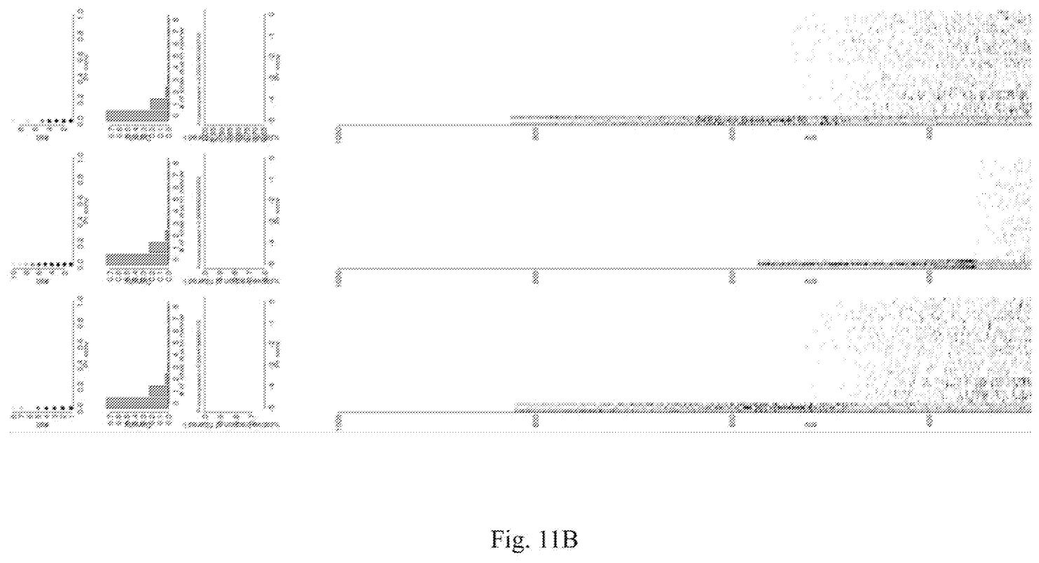

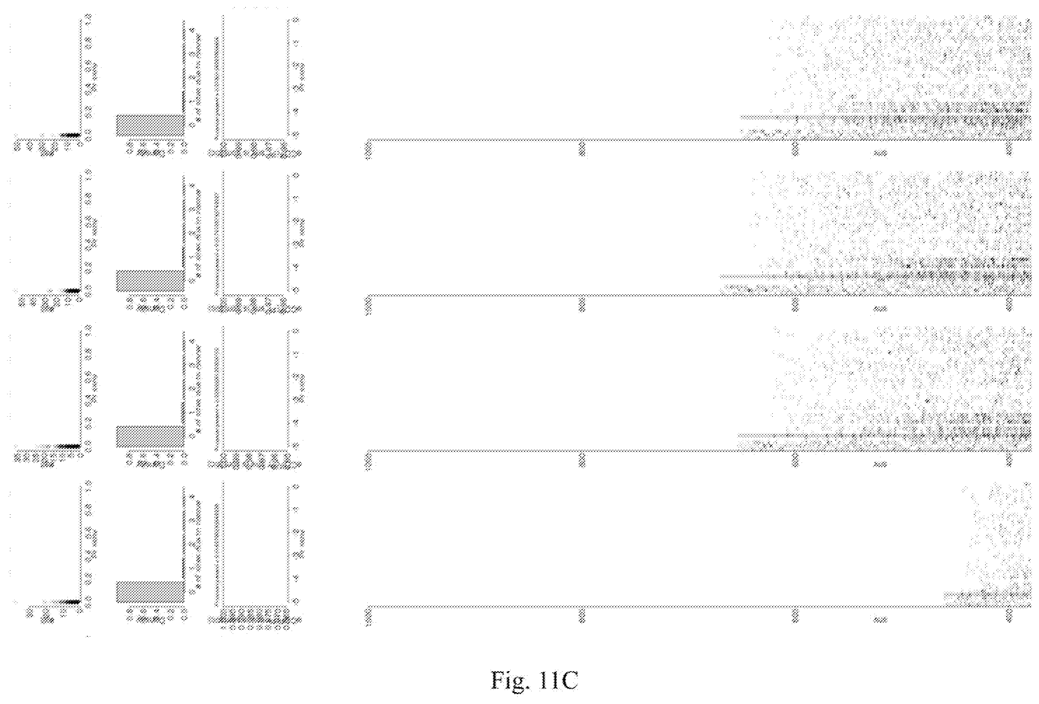

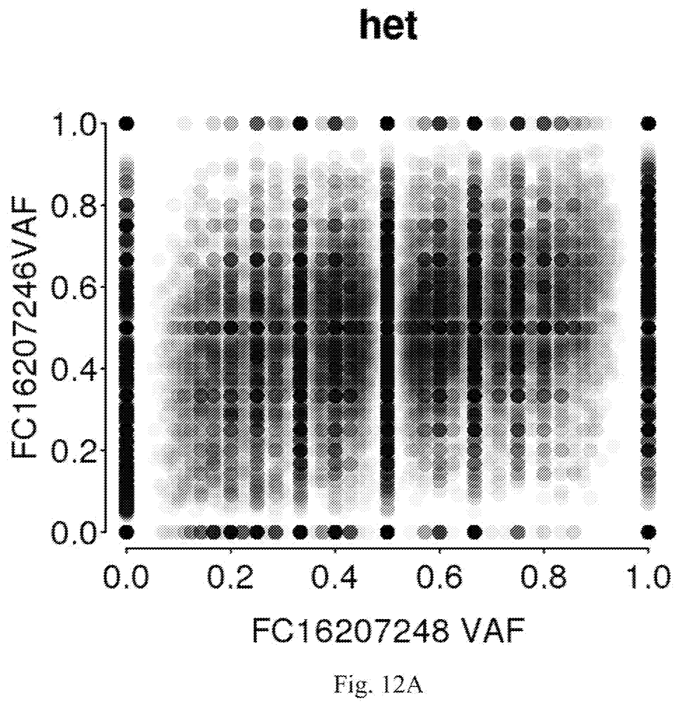

Referring now to FIGS. 9A-B, data illustrating robust detection of subclinical lung cancer in a patient according to embodiments of the present disclosure is provided. In FIG. 10A, each column represents the comparison of one sample to 36 cfDNA and gDNA WGS samples. In each column, the 2nd and 4th rows are relevant. The 4th (elongated) row depicts the number of alternate reads in samples (x-axis) vs. sites (y-axis). Shade indicates the number of reads supporting the site, from 0 (white) to 5 or more (black). The sites shown in each comparison must have been detected as somatic variants in both the test sample and in the test patient. In each comparison, sites are sorted by the probability that they represent somatic variants (sorted to the top of the y-axis of each plot), vs. sites w. high error rates (sorted to the bottom). The test sample increments by column, ranging from 1 to 36 (the total number of samples sequenced). The first two samples are from a single test patient.

FIG. 9B is a detail view of the inset of FIG. 9A. The inset shows the posterior distribution over the number of variant sites that are due to cancer detected in the test sample (second row). Only samples from the test patient show appreciable non-zero probability of cancer detection. Note that data from the test sample is withheld from this calculation if it is from the test patient.

Referring to FIGS. 10A-E, each plot shows a 2D scatter plot of the variant-allele fraction (the fraction of total reads reporting the alternate allele at a given locus; VAF). Sample 2 represents a post-relapse high-tumor-shed state, whereas sample 1 was obtained at a timepoint in which no cancer was detectible in the patient, either by imaging, deep DNA sequencing (ddPCR), or other biomarker assays. Both samples are plasma-derived cfDNA.

Each column corresponds to classification of variants as inherited (het, AA, or BB), or potentially somatic (outlier). The variants labeled as AA may also comprise error-prone sites identified using a model trained on 30 other cfDNA WGS samples. The last column shows all variants together will most-likely class indicated by color. The presence of a large number of somatic mutations (blue points >0) in all both samples demonstrates the robust detection somatic clones. These variants are heavily enriched in smoking-associated mutations (not shown).

Referring now to FIGS. 11A-C, data illustrating robust detection of breast cancer in a patient according to embodiments of the present disclosure is provided. In FIG. 11A, each column represents the comparison of one sample to 36 cfDNA and gDNA WGS samples. In each column, the 2nd and 4th rows are relevant. The 4th (elongated) row depicts the number of alternate reads in samples (x-axis) vs. sites (y-axis). Shade indicates the number of reads supporting the site, from 0 (white) to 5 or more (black). The sites shown in each comparison must have been detected as somatic variants in both the test sample and in the test patient. In each comparison, sites are sorted by the probability that they represent somatic variants (sorted to the top of the y-axis of each plot), vs. sites w. high error rates (sorted to the bottom). The test sample increments by column, ranging from 1 to 36 (the total number of samples sequenced). The first three samples are from a single test patient.

FIGS. 11B-C are detail views of FIG. 11A, with FIG. 11B covering columns 1-3 and FIG. 11C covering columns 4-7. The inset shows the posterior distribution over the number of variant sites that are due to cancer detected in the test sample (2nd row). Only samples from the test patient show appreciable non-zero probability of cancer detection. Note that data from the test sample is withheld from this calculation if it is from the test patient.

Here, the three samples from the test patient represent cfDNA samples from plasma and cerebrospinal fluid of a patient with brain metastasis and leptomeningeal disease from breast cancer.

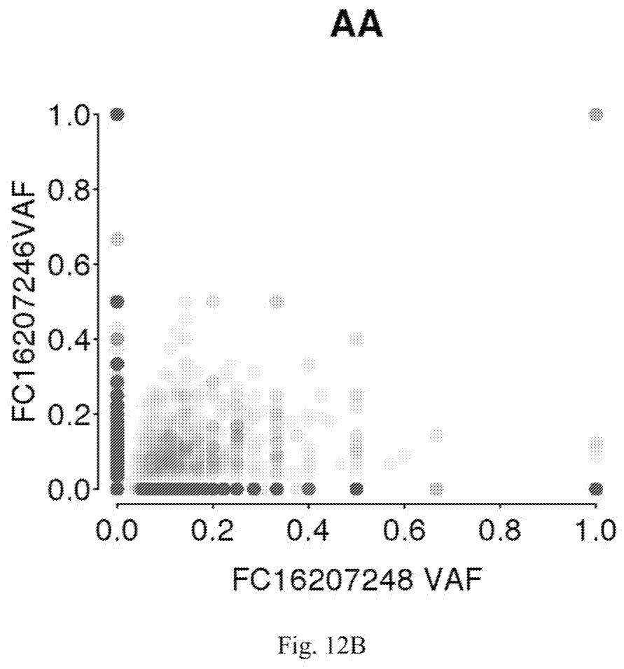

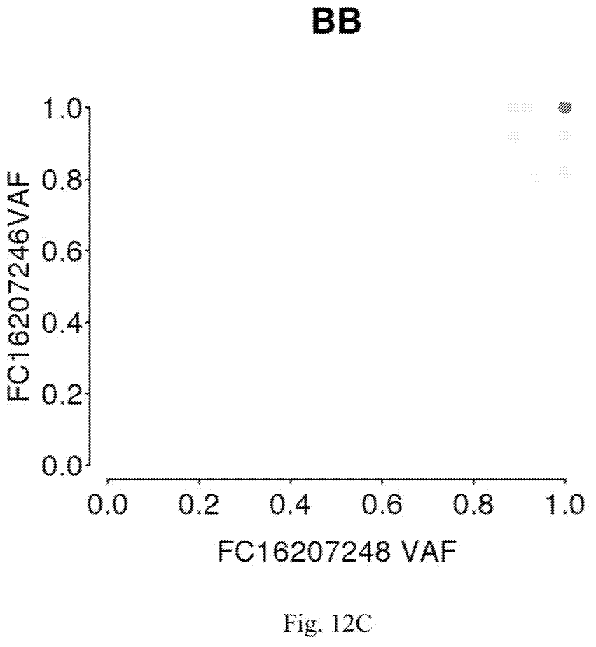

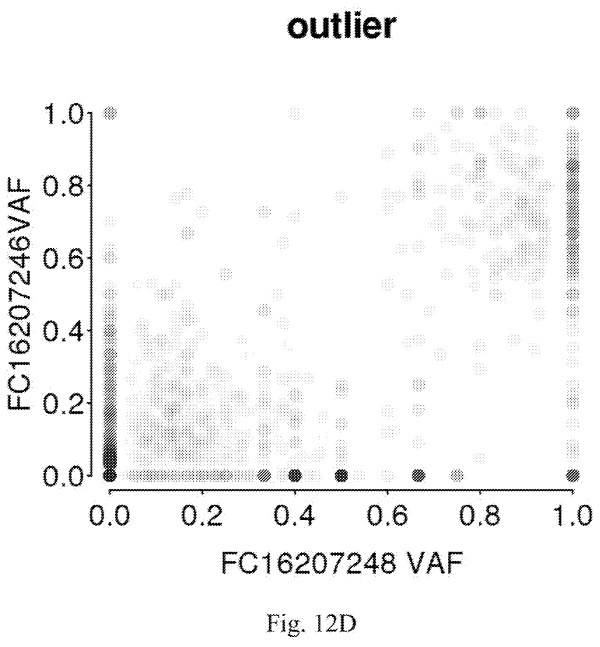

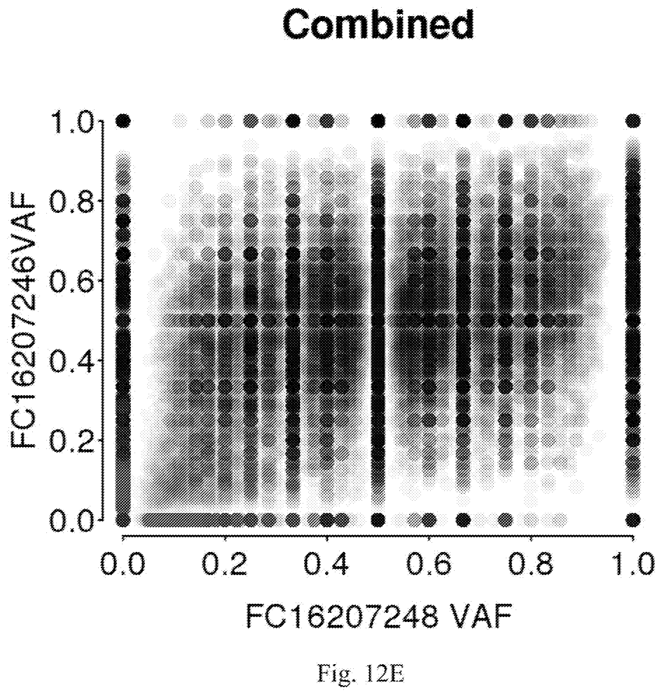

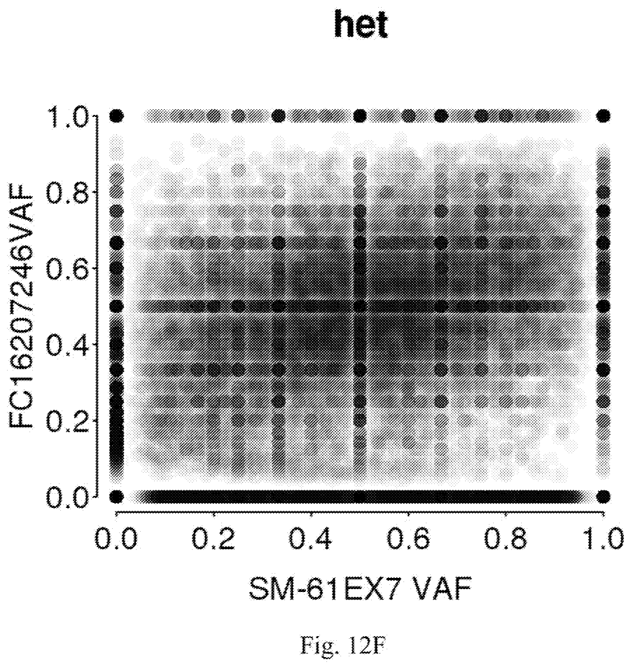

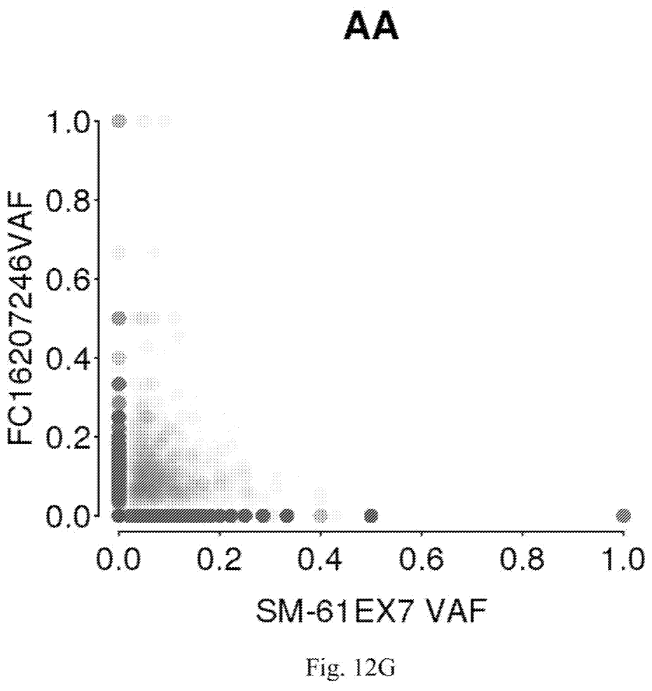

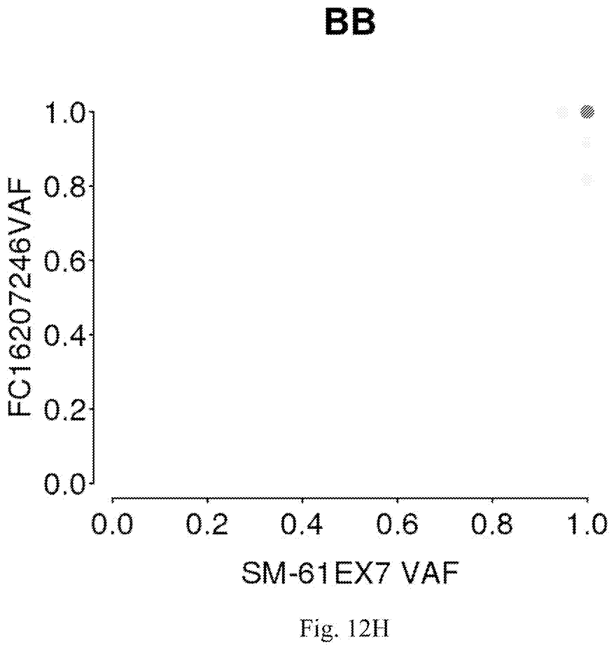

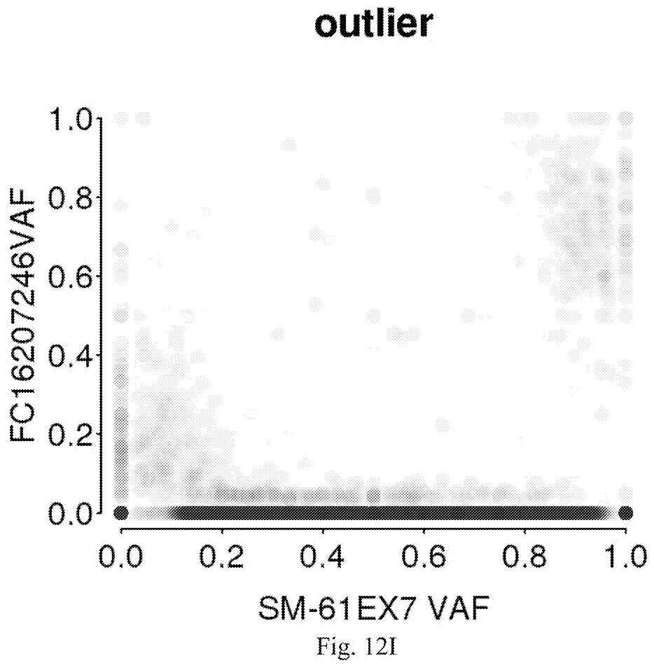

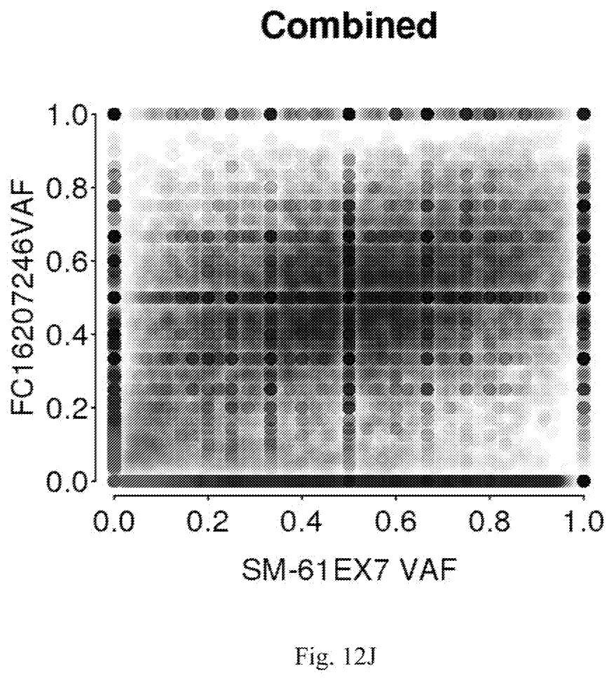

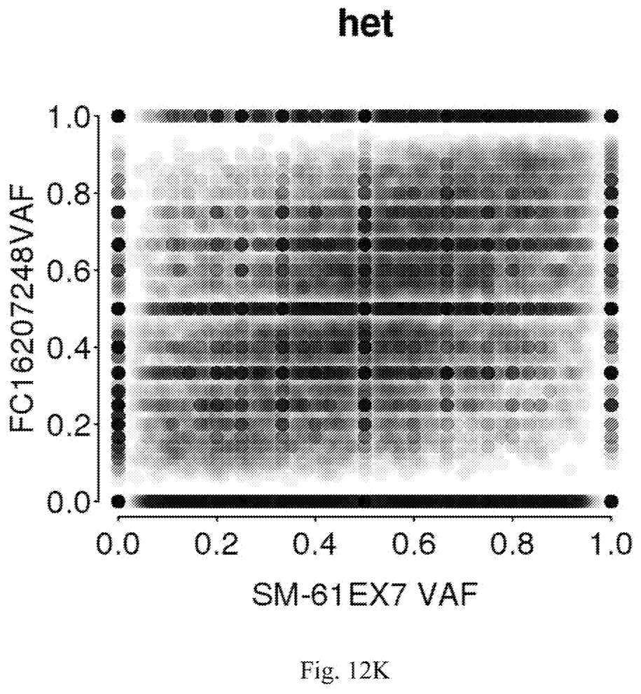

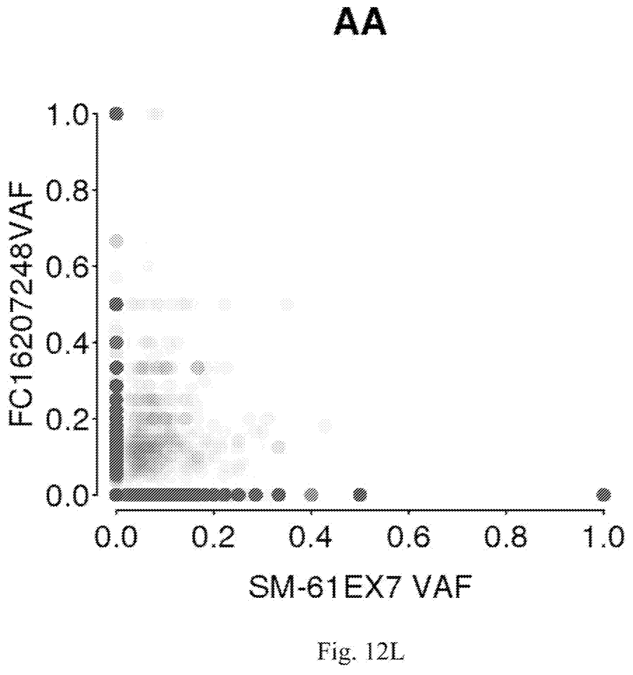

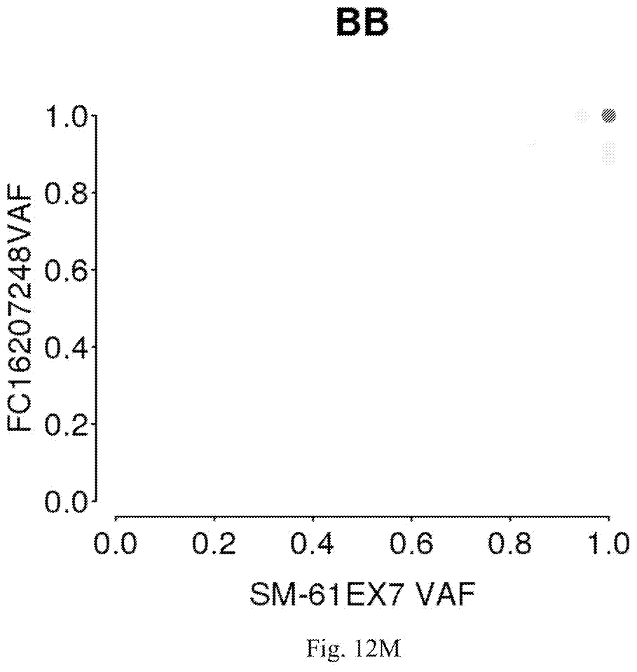

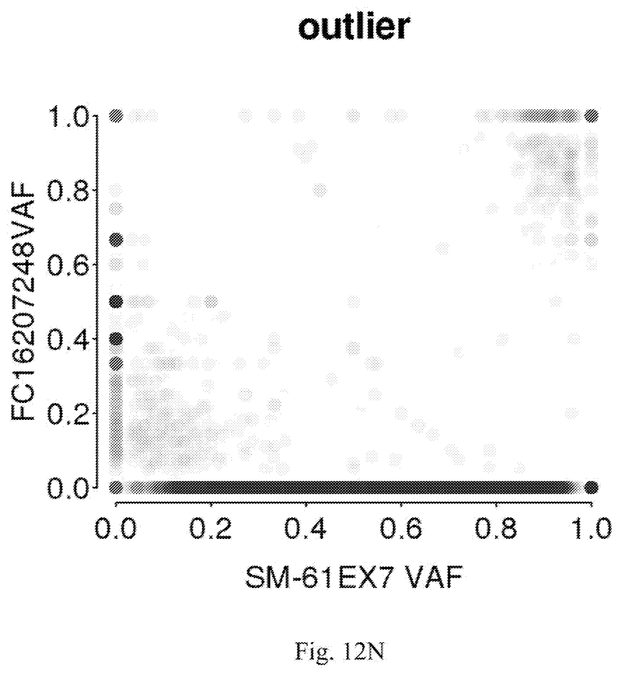

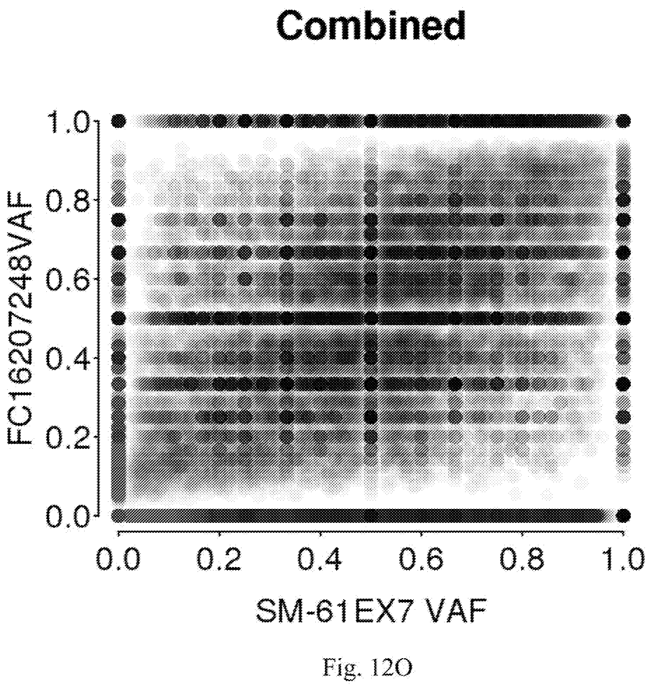

Referring to FIG. 12A-O, each plot shows a 2D scatter plot of the variant-allele fraction (the fraction of total reads reporting the alternate allele at a given locus; VAF). Each row (FIGS. 12A-E; F-J; and K-O) corresponds to a comparison between two samples sequenced from a melanoma test patient. Sample SM-61EX7 is from tissue obtained from surgical resection. The other two samples are from plasma-derived cfDNA.

Each column corresponds to classification of variants as inherited (het, AA, or BB), or potentially somatic (outlier). The last column (FIGS. 12E, J, O) shows all variants together will most-likely class indicated by color. The variants labeled as AA may also comprise error-prone sites identified using a model trained on 30 other cfDNA WGS samples. The presence of a large number of somatic mutations (blue points >0) in all samples demonstrates the robust detection somatic clones. These variants are heavily enriched in UV-associated mutations (not shown).

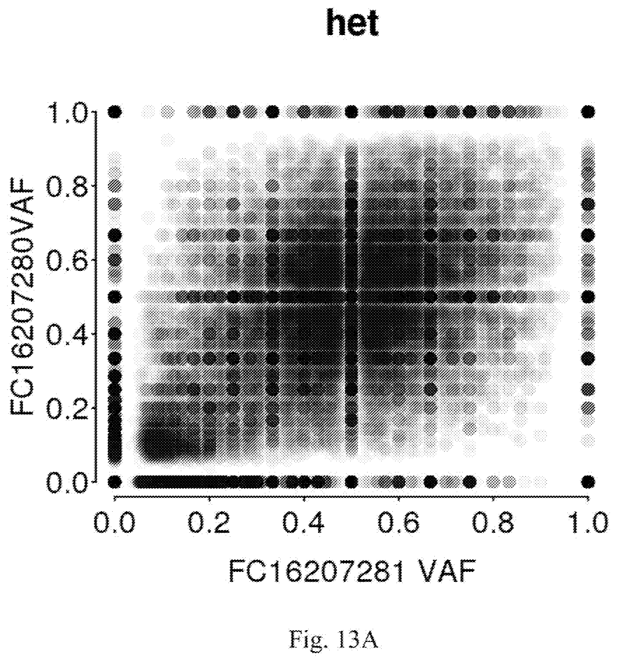

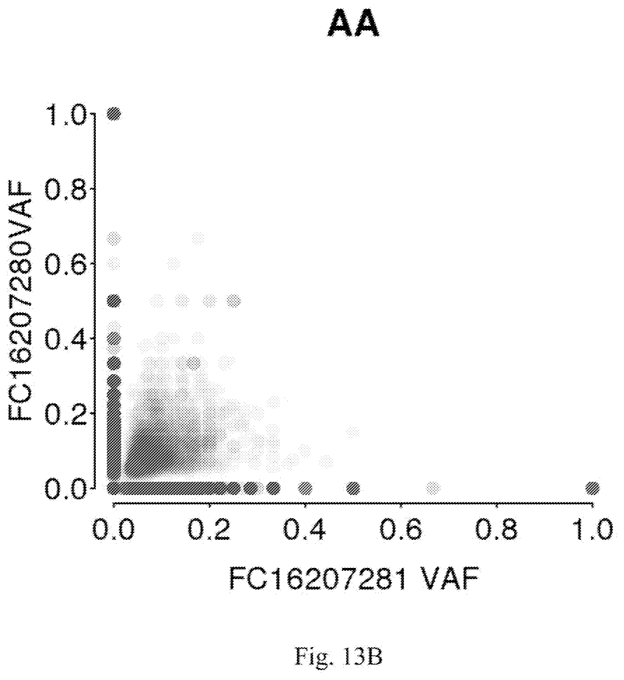

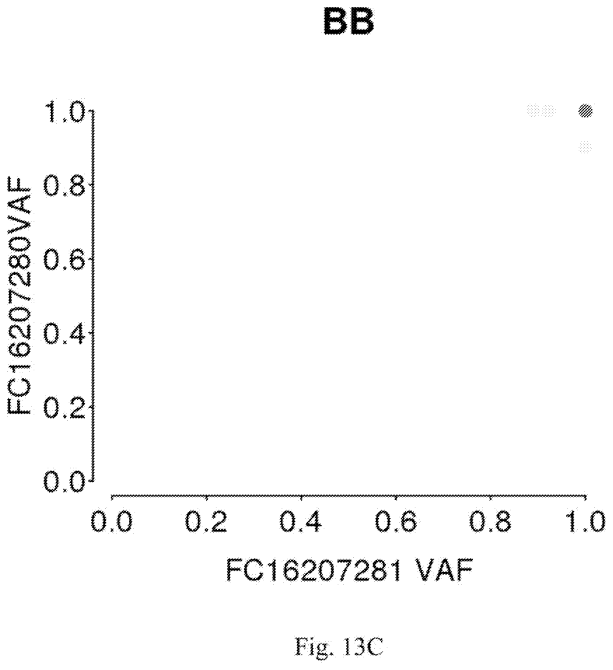

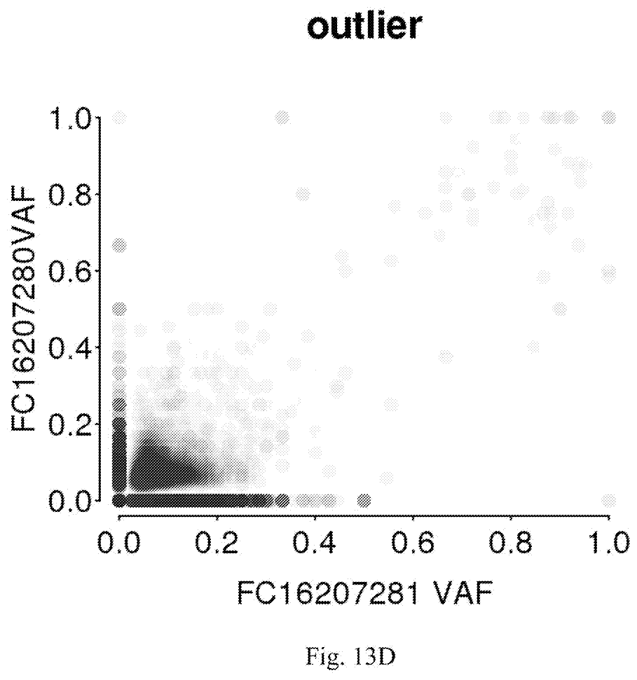

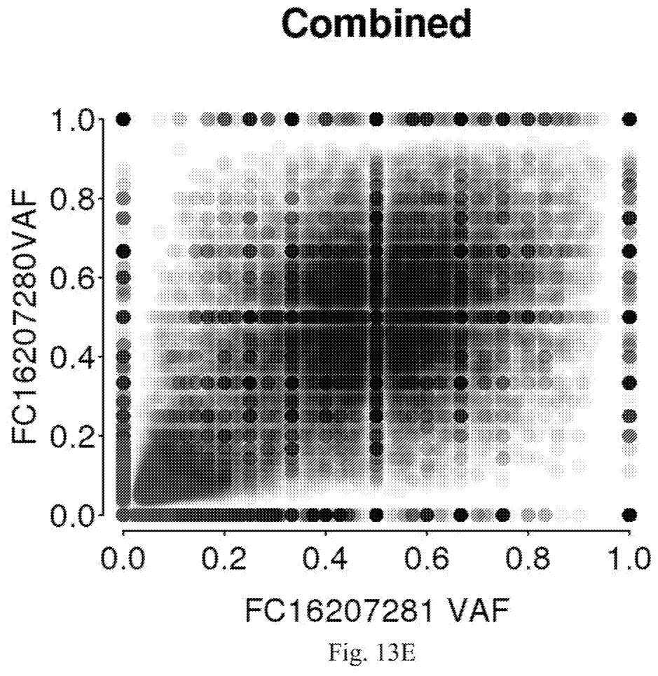

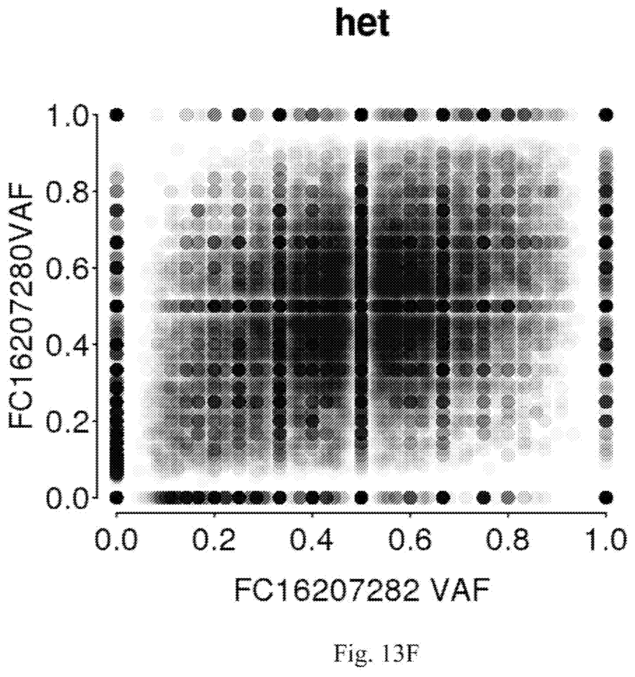

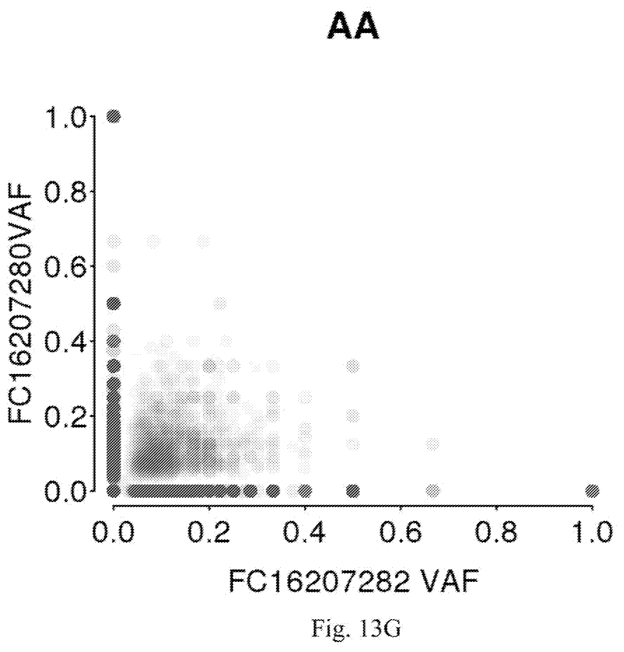

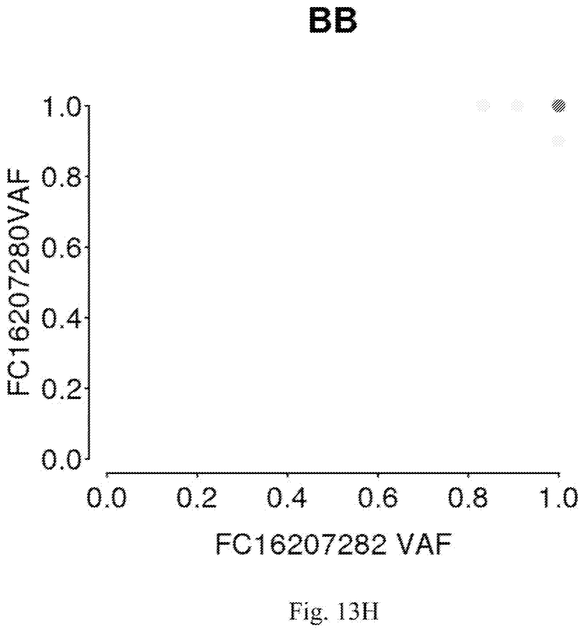

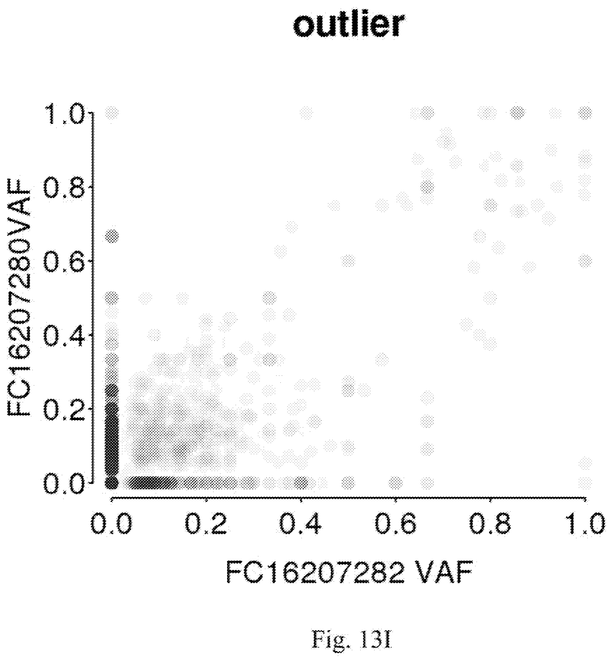

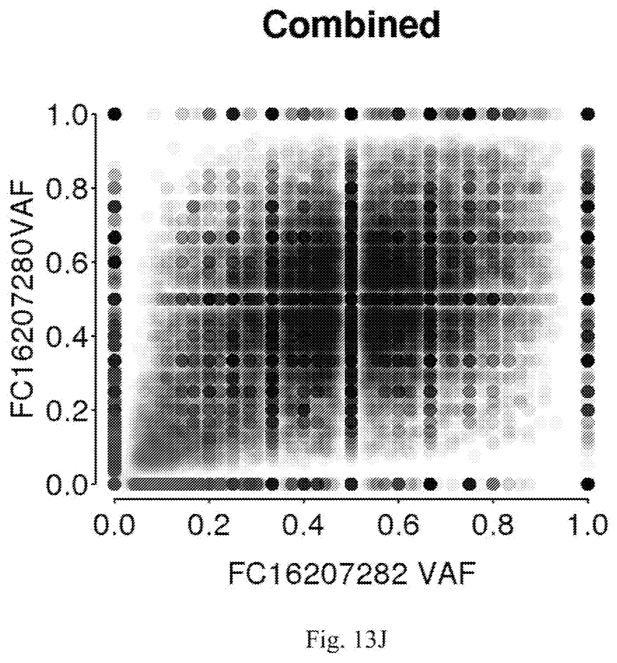

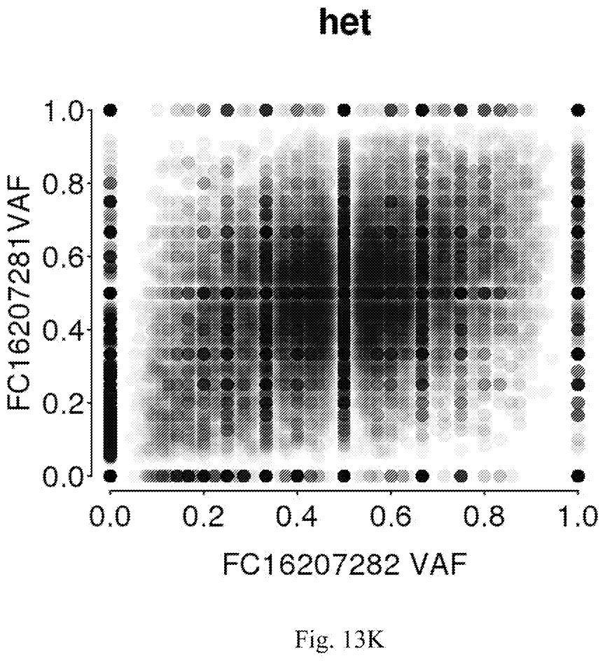

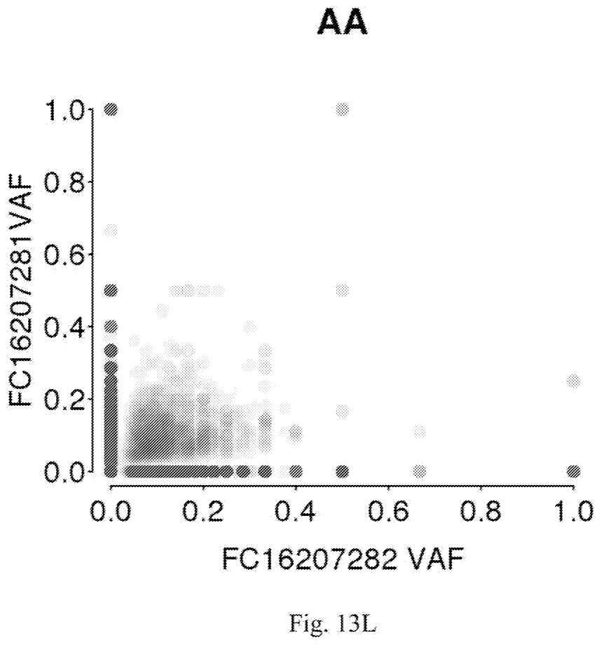

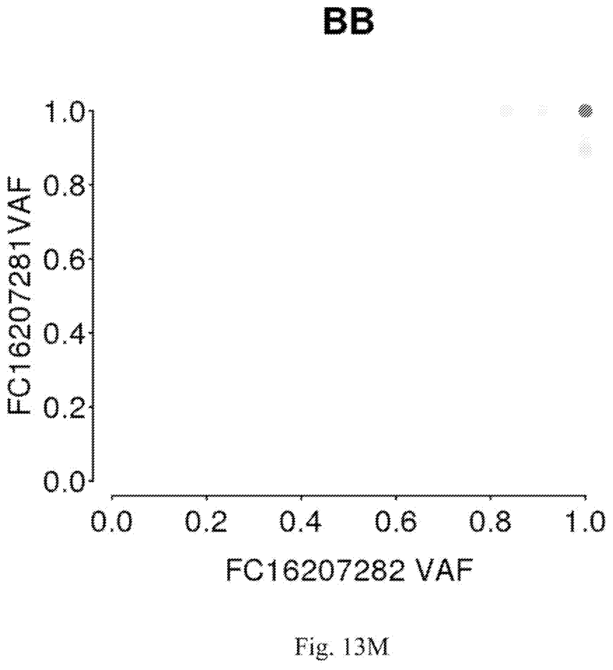

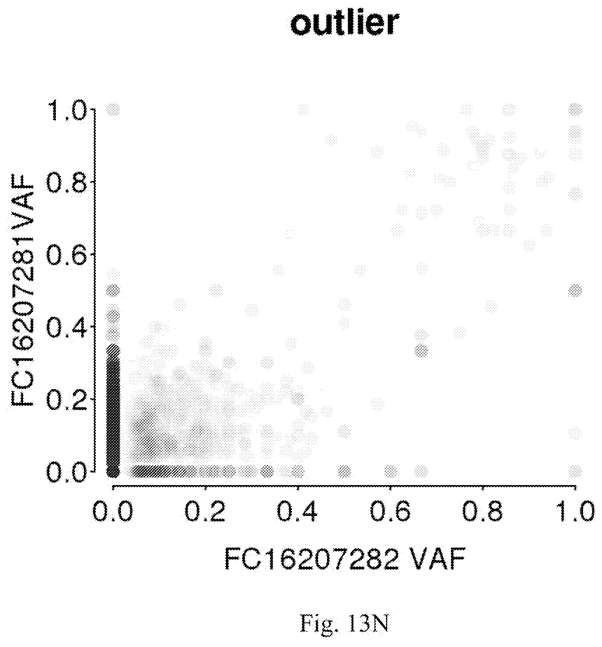

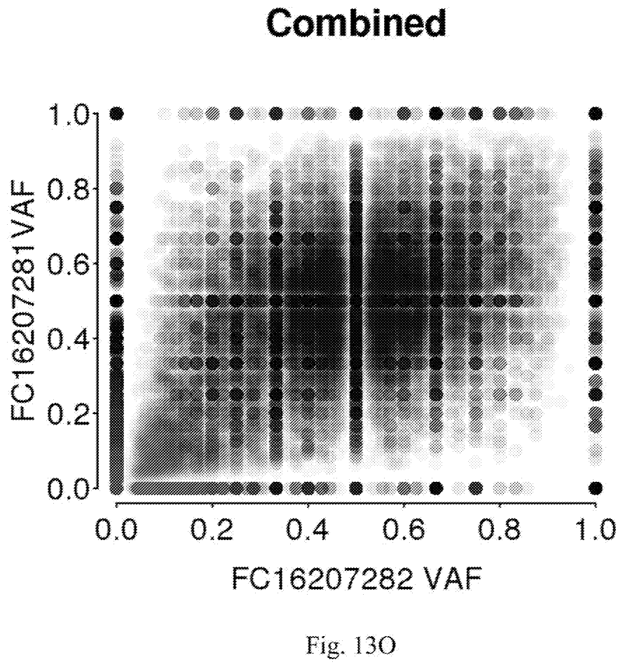

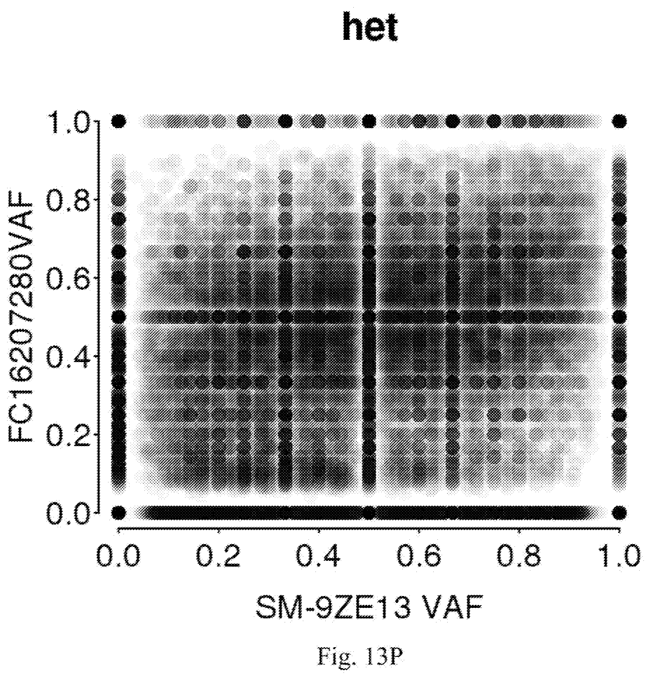

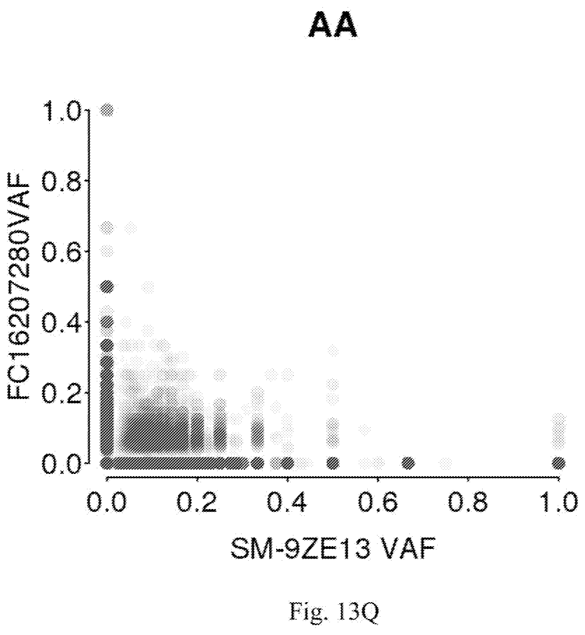

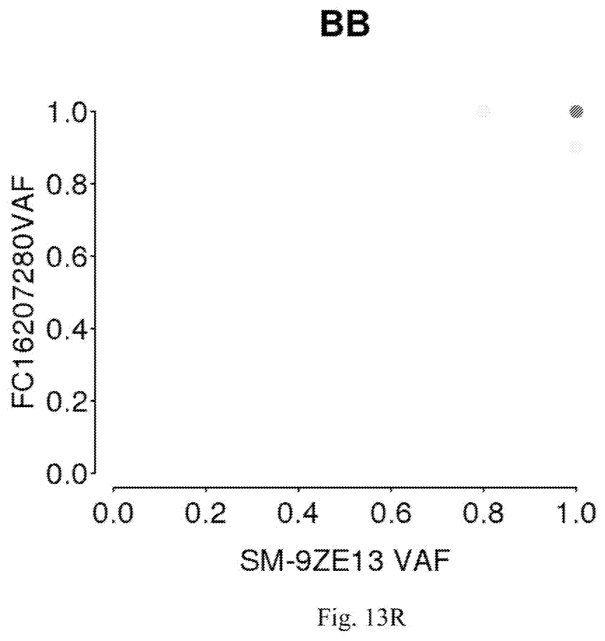

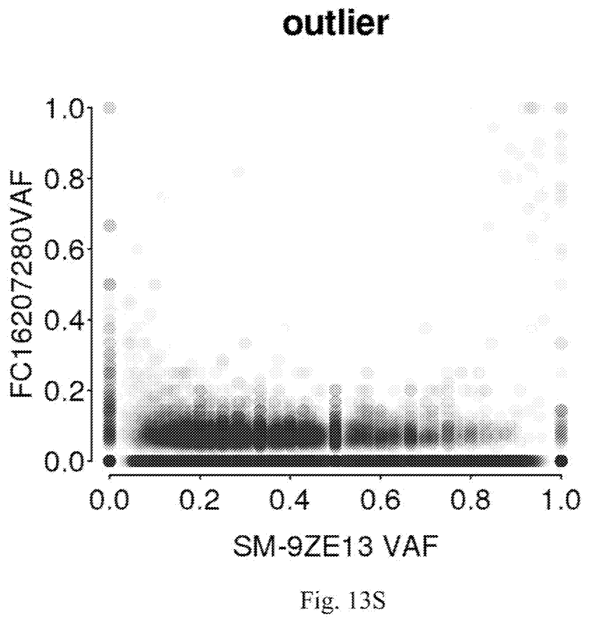

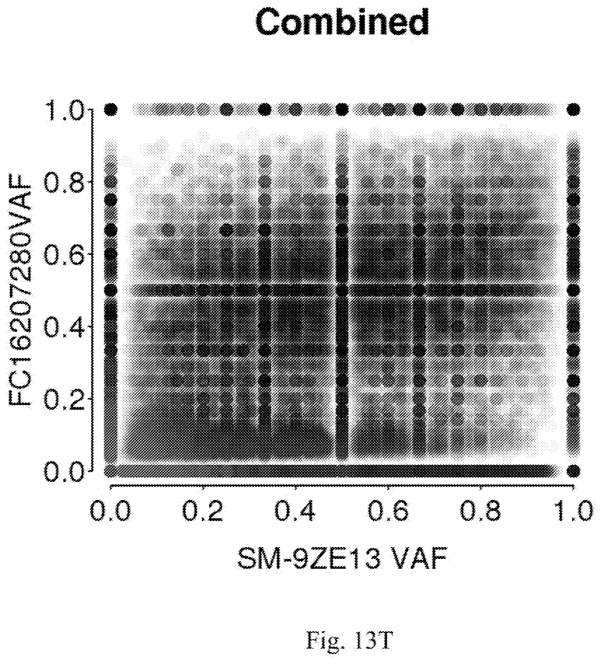

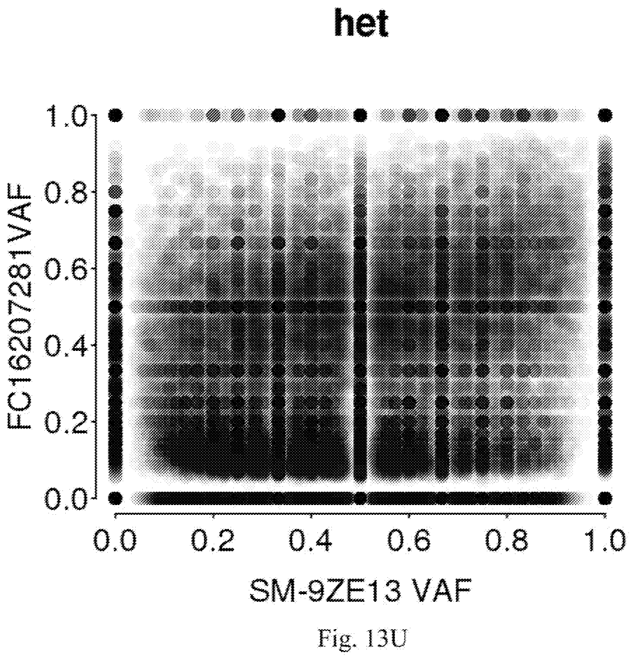

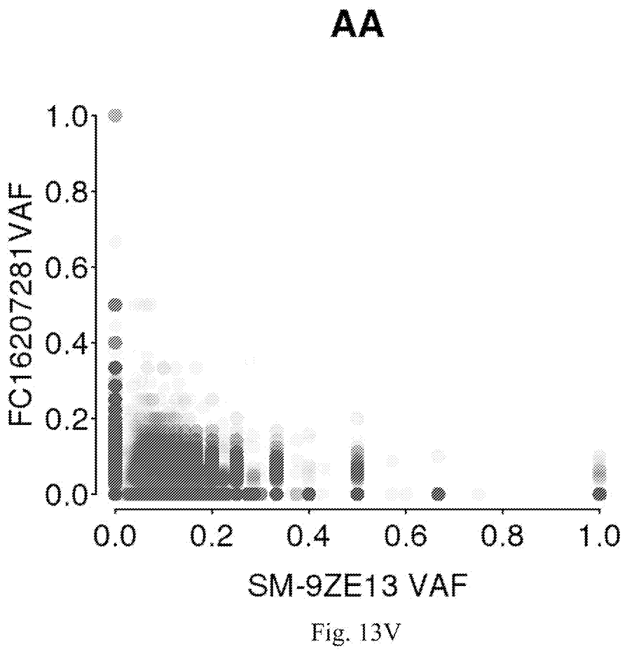

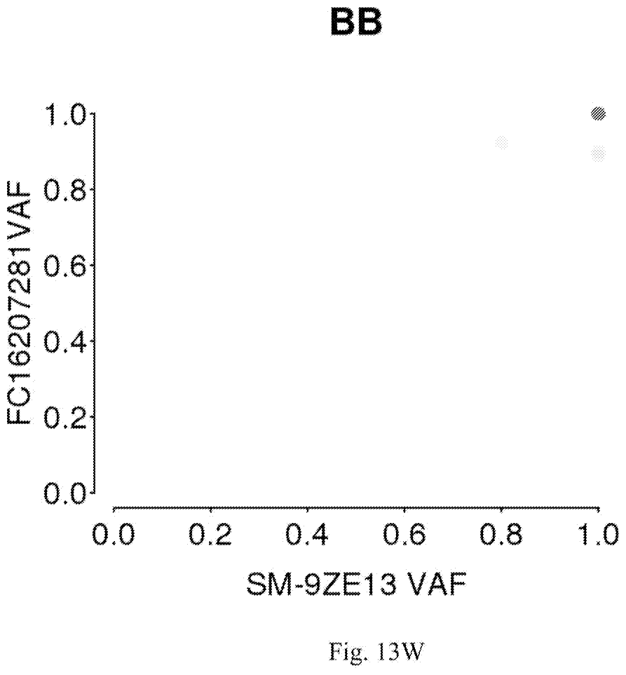

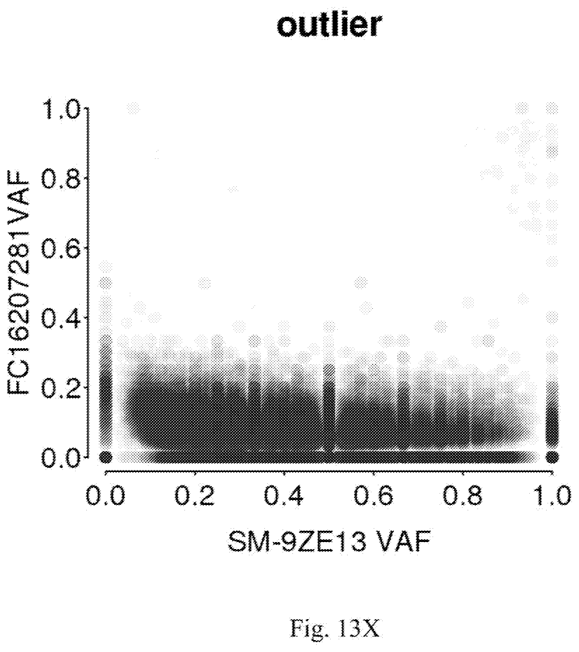

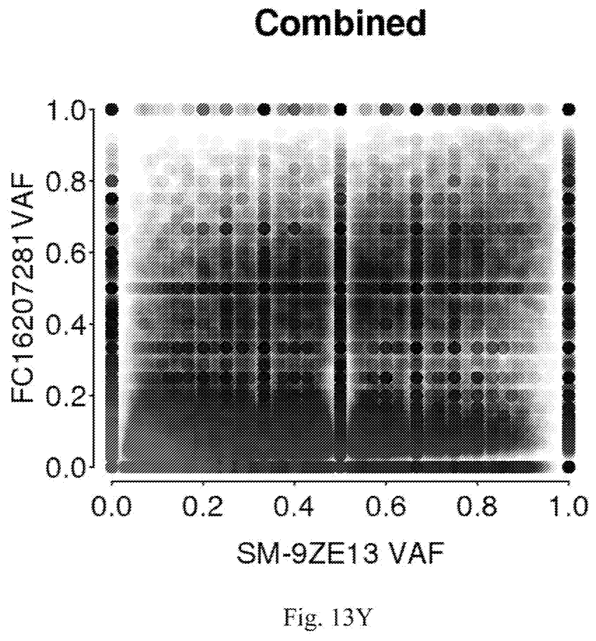

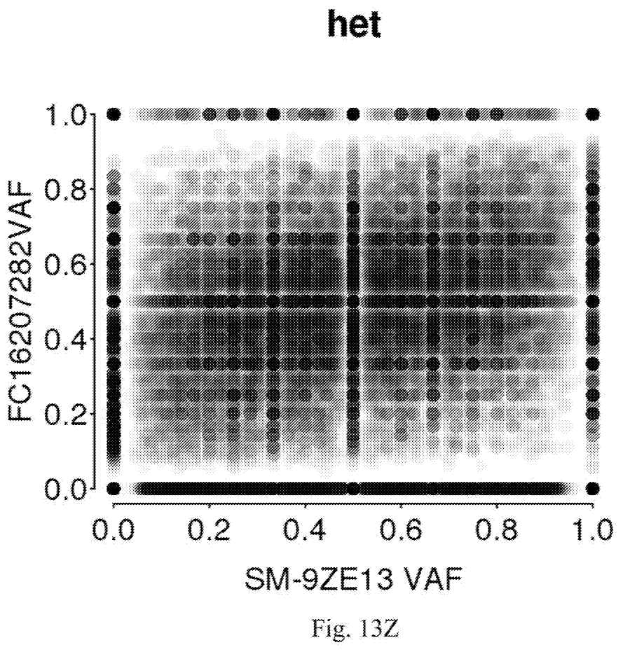

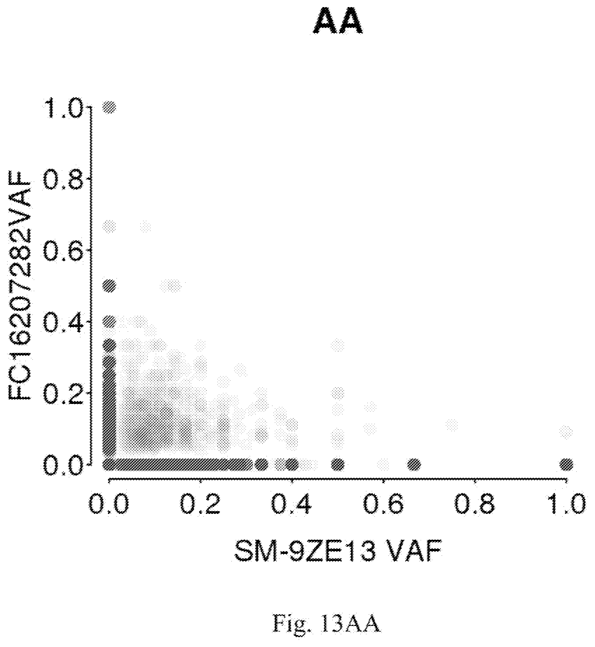

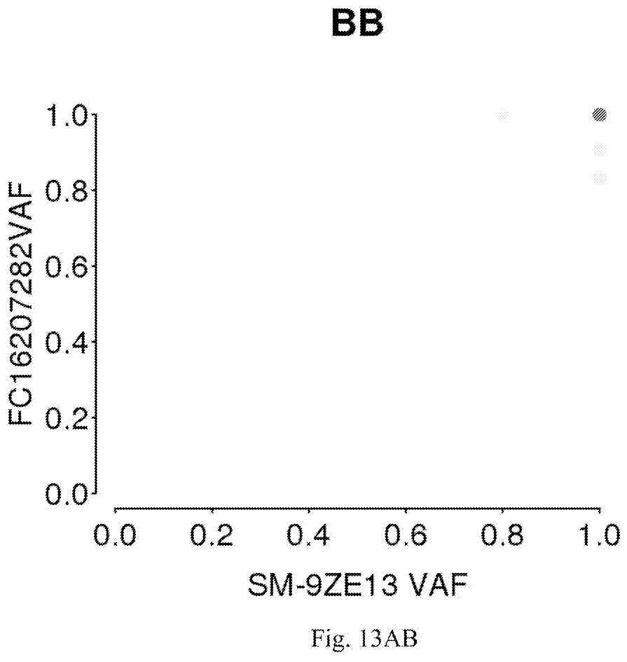

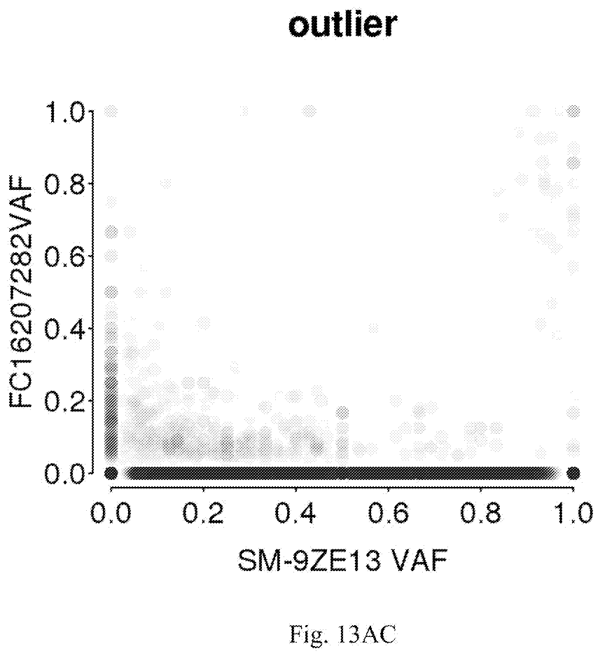

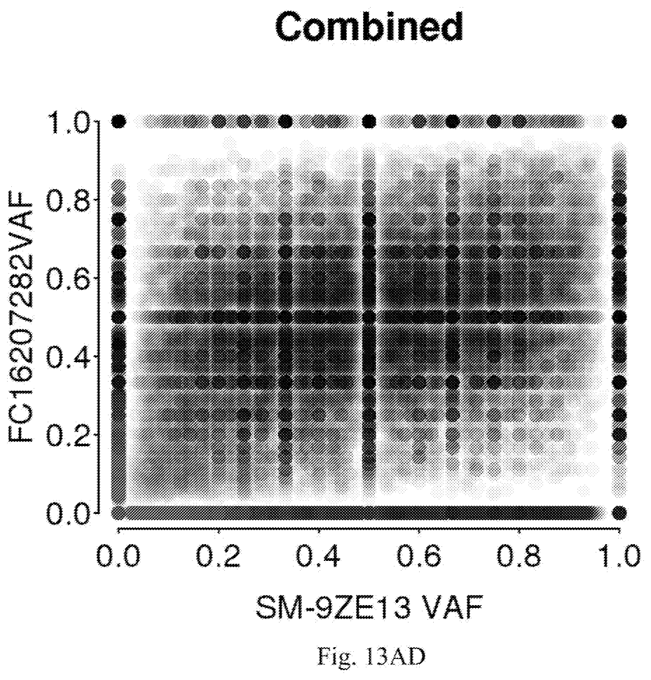

Referring to FIGS. 13A-AD, each plot shows a 2D scatter plot of the variant-allele fraction (the fraction of total reads reporting the alternate allele at a given locus; VAF). Each row (FIGS. 13A-E; F-J; K-O; P-T, U-Y; Z-AD) corresponds to a comparison between two samples sequenced from a melanoma test patient. Sample SM-9ZE13 is from tissue obtained from surgical resection. The other two samples are from plasma-derived cfDNA.

Each column corresponds to classification of variants as inherited (het, AA, or BB), or potentially somatic (outlier). The variants labeled as AA may also comprise error-prone sites identified using a model trained on 30 other cfDNA WGS samples. The last column (FIGS. 13E, J, O, T, Y, AD) shows all variants together will most-likely class indicated by color. The presence of a large number of somatic mutations (blue points >0) in all samples demonstrates the robust detection somatic clones. These variants are heavily enriched in UV-associated mutations (not shown).

In various embodiments, fingerprint mutation detection in a subclinical cancer sample is distinguished from sequencing error using mutational signatures and phylogenetic analysis.

In various embodiments, the mutational signature model is extended beyond 96 single-base-substitution classes to include insertions and deletions, as well as di-nucleotide and multi-nucleotide frequencies associated with mutational processes active in human cancers.

In various embodiments, a statistical model is constructed to estimate the sequencing error rate at any location in the genome, based on local sequence composition and additional covariates. This model also utilizes ground-truth sequencing errors identified by overlapping paired-end reads.

Various embodiments do not rely on a germline gDNA reference sample for a given patient—the multi-sample phylogenetic classification of variants as germline vs. somatic based on VAF combined with the mutational signatures classifier based on sequence context obviates the need for such a reference sample. This is advantageous, as such reference samples are often difficult to obtain, and even when they are available, may be misleading as cancer-derived cfDNA may contaminate the gDNA.

In various embodiments, the strategy of paired whole-genome sequencing is applied where DNA sequencing libraries are constructed using dual-index barcodes that uniquely identify DNA-fragments in the input sample. Detection of cancer-fingerprint mutations in the whole-genome sequencing of a test sample may subsequently be followed-up by ultradeep sequencing of the same library, with targeted hybrid-capture of genomic regions harboring detected fingerprint mutations. This provides another level of validation that the detected fingerprint mutation was not due to sequencing error, and can substantially increase the sensitivity of detection, while not compromising specificity.

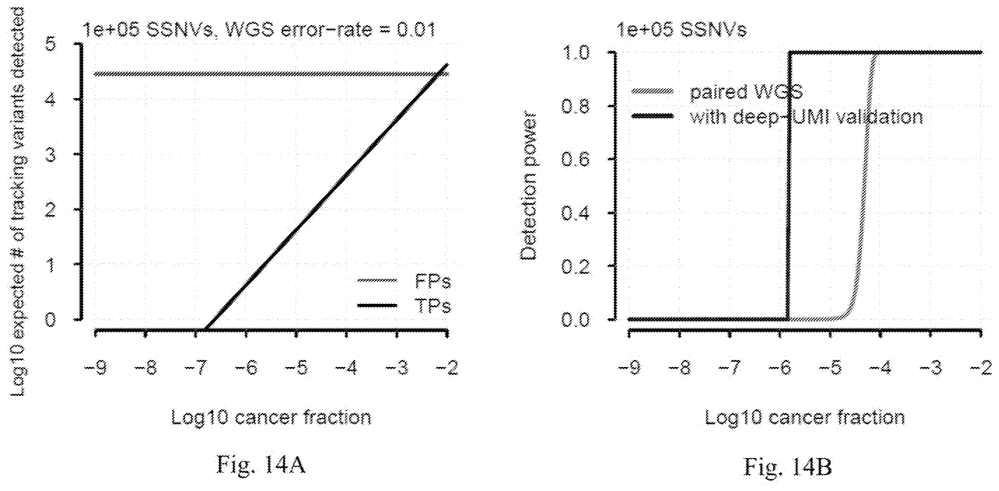

FIG. 14A shows the expected number of fingerprint somatic single-nucleotide substitutions (SSNVs) detected in a theoretical MRD liquid biopsy cfDNA sample, as a function of cancer fraction. The total number of tracking fingerprint substitutions was 100,000. 3.6 ng input DNA was modeled, resulting in 85× unique coverage, with base-level WGS sequencing error rate was assumed to be 1/100. Detected tracking mutations are designated as true-positive (TPs) if they were derived from cancer in the simulation, or false-positives (FPs) if they were detected due to sequencing error.

FIG. 14B shows detection power as a function of cancer fraction using the paired WGS approach, or with additional validation sequencing of all detected mutations using deep targeted UMI sequencing in the original library. Power was calculated using a binomial model and first considering the scenario where all detected fingerprint mutations were false positives in the test WGS sample. In this case, the number of these errors that also produce errors in the followup UMI validation was modeled using a binomial distribution with N equal to the expected number of false-positives and p equal to the deep UMI-sequencing error rate modeled as 1e-4, which accounts for both PCR errors in the 1st cycle of library construction and read-mapping error. The quantile k of this distribution was computed corresponding to 1—the FDR rate—here 1e-4. The number of correctly validated mutations was modeled as a binomial distribution with N equal to the number of expected true positives detected in the paired WGS, and p equal to 1—the UMI-error rate. Power was computed as the probability of detecting at least k fingerprint mutations, which would cause rejection of the null-hypothesis that all deep-UMI-validated mutations were due to errors at the desired FDR.

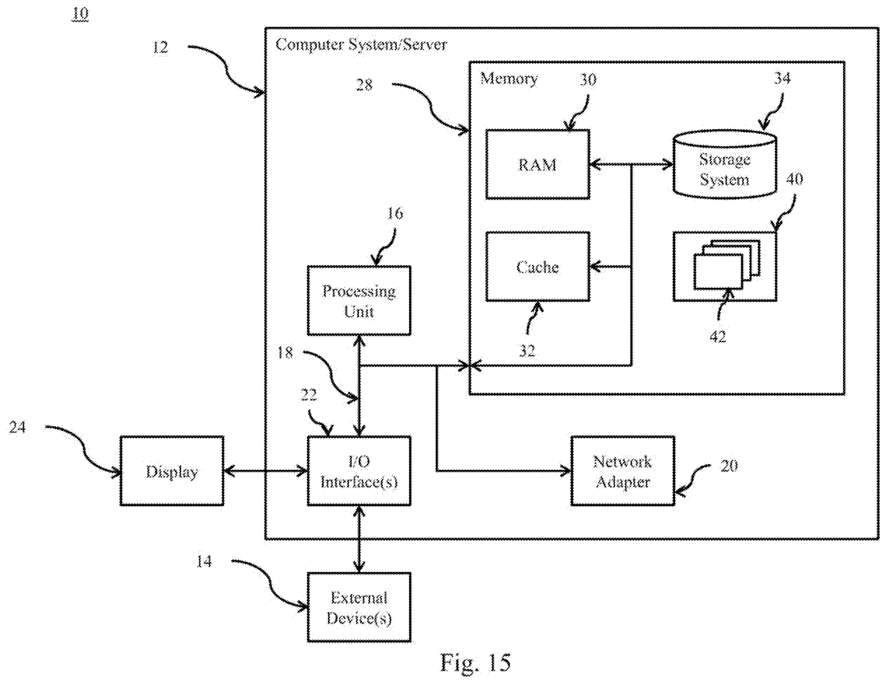

Referring now to FIG. 15, a schematic of an example of a computing node is shown. Computing node 10 is only one example of a suitable computing node and is not intended to suggest any limitation as to the scope of use or functionality of embodiments described herein. Regardless, computing node 10 is capable of being implemented and/or performing any of the functionality set forth hereinabove.

In computing node 10 there is a computer system/server 12, which is operational with numerous other general purpose or special purpose computing system environments or configurations. Examples of well-known computing systems, environments, and/or configurations that may be suitable for use with computer system/server 12 include, but are not limited to, personal computer systems, server computer systems, thin clients, thick clients, handheld or laptop devices, multiprocessor systems, microprocessor-based systems, set top boxes, programmable consumer electronics, network PCs, minicomputer systems, mainframe computer systems, and distributed cloud computing environments that include any of the above systems or devices, and the like.

Computer system/server 12 may be described in the general context of computer system-executable instructions, such as program modules, being executed by a computer system. Generally, program modules may include routines, programs, objects, components, logic, data structures, and so on that perform particular tasks or implement particular abstract data types. Computer system/server 12 may be practiced in distributed cloud computing environments where tasks are performed by remote processing devices that are linked through a communications network. In a distributed cloud computing environment, program modules may be located in both local and remote computer system storage media including memory storage devices.

As shown in FIG. 15, computer system/server 12 in computing node 10 is shown in the form of a general-purpose computing device. The components of computer system/server 12 may include, but are not limited to, one or more processors or processing units 16, a system memory 28, and a bus 18 that couples various system components including system memory 28 to processor 16.

Bus 18 represents one or more of any of several types of bus structures, including a memory bus or memory controller, a peripheral bus, an accelerated graphics port, and a processor or local bus using any of a variety of bus architectures. By way of example, and not limitation, such architectures include Industry Standard Architecture (ISA) bus, Micro Channel Architecture (MCA) bus, Enhanced ISA (EISA) bus, Video Electronics Standards Association (VESA) local bus, Peripheral Component Interconnect (PCI) bus, Peripheral Component Interconnect Express (PCIe), and Advanced Microcontroller Bus Architecture (AMBA).

Computer system/server 12 typically includes a variety of computer system readable media. Such media may be any available media that is accessible by computer system/server 12, and it includes both volatile and non-volatile media, removable and non-removable media.

System memory 28 can include computer system readable media in the form of volatile memory, such as random access memory (RAM) 30 and/or cache memory 32. Computer system/server 12 may further include other removable/non-removable, volatile/non-volatile computer system storage media. By way of example only, storage system 34 can be provided for reading from and writing to a non-removable, non-volatile magnetic media (not shown and typically called a “hard drive”). Although not shown, a magnetic disk drive for reading from and writing to a removable, non-volatile magnetic disk (e.g., a “floppy disk”), and an optical disk drive for reading from or writing to a removable, non-volatile optical disk such as a CD-ROM, DVD-ROM or other optical media can be provided. In such instances, each can be connected to bus 18 by one or more data media interfaces. As will be further depicted and described below, memory 28 may include at least one program product having a set (e.g., at least one) of program modules that are configured to carry out the functions of embodiments of the disclosure.

Program/utility 40, having a set (at least one) of program modules 42, may be stored in memory 28 by way of example, and not limitation, as well as an operating system, one or more application programs, other program modules, and program data. Each of the operating system, one or more application programs, other program modules, and program data or some combination thereof, may include an implementation of a networking environment. Program modules 42 generally carry out the functions and/or methodologies of embodiments as described herein.

Computer system/server 12 may also communicate with one or more external devices 14 such as a keyboard, a pointing device, a display 24, etc.; one or more devices that enable a user to interact with computer system/server 12; and/or any devices (e.g., network card, modem, etc.) that enable computer system/server 12 to communicate with one or more other computing devices. Such communication can occur via Input/Output (I/O) interfaces 22. Still yet, computer system/server 12 can communicate with one or more networks such as a local area network (LAN), a general wide area network (WAN), and/or a public network (e.g., the Internet) via network adapter 20. As depicted, network adapter 20 communicates with the other components of computer system/server 12 via bus 18. It should be understood that although not shown, other hardware and/or software components could be used in conjunction with computer system/server 12. Examples, include, but are not limited to: microcode, device drivers, redundant processing units, external disk drive arrays, RAID systems, tape drives, and data archival storage systems, etc.

The present disclosure may be embodied as a system, a method, and/or a computer program product. The computer program product may include a computer readable storage medium (or media) having computer readable program instructions thereon for causing a processor to carry out aspects of the present disclosure.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present disclosure may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Smalltalk, C++ or the like, and conventional procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present disclosure.

Aspects of the present disclosure are described herein with reference to flowchart illustrations and/or block diagrams of methods, apparatus (systems), and computer program products according to embodiments of the disclosure. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present disclosure. In this regard, each block in the flowchart or block diagrams may represent a module, segment, or portion of instructions, which comprises one or more executable instructions for implementing the specified logical function(s). In some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

The descriptions of the various embodiments of the present disclosure have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

What is claimed is:

1. A method of analyzing a sample comprising a plurality of polynucleotides, the method comprising:
receiving a plurality of sequences of the plurality of polynucleotides;
providing the plurality of sequences to a trained classifier, the trained classifier adapted to accept a plurality of sequences and output a class label indicative of the presence of a somatic variant within the plurality of sequences;
receiving from the trained classifier a class label indicative of the presence of a somatic clone within the plurality of sequences.

2. The method of claim 1, further comprising:
based on the label, indicating whether a cancer clone is present in the sample.

3. The method of claim 1, further comprising:
based on the label, indicating whether a clonal expansion is present in the sample.

4. The method of claim 1, wherein the label has an associated probability.

5. The method of claim 1, further comprising:
providing clinical data to the trained classifier, the clinical data being related to an originator of the sample.

6. The method of claim 1, wherein the clinical data comprises an indication of smoking by the originator of the sample.

7. The method of claim 1, wherein the clinical data comprises family history of the originator of the sample.

8. The method of claim 1, further comprising:
providing population data to the trained classifier, the population data being related to an originator of the sample.

9. The method of claim 1, wherein the plurality of sequences comprise a plurality of somatic variants.

10. The method of claim 1, wherein the trained classifier comprises an artificial neural network.

11. The method of claim 1, wherein the trained classifier comprises a regression model.

12. The method of claim 1, wherein the trained classifier comprises a random decision forest.

13. The method of claim 1, wherein the trained classifier comprises an SVM.

14. The method of claim 10, wherein the artificial neural network is a convolutional neural network.

15. The method of claim 10, wherein the artificial neural network is a recurrent neural network.

16. The method of claim 1, wherein the sample comprises blood.

17. The method of claim 1, wherein the sample comprises cerebrospinal fluid.

18. The method of claim 1, wherein the plurality of polynucleotides comprises DNA.

19. The method of claim 1, wherein the plurality of polynucleotides comprises methylated DNA.

20. The method of claim 1, further comprising:
providing fragment lengths of the plurality of sequences to the trained classifier.

21. The method of claim 1, wherein the plurality of polynucleotides comprises RNA.

22. The method of claim 1, wherein the sequencing is at a depth of 100× or less.

23. The method of claim 1, wherein the sequencing is at a depth of 85× or less.

24. The method of claim 1, wherein the sequencing is at a depth of about 20× to about 85×.

25. The method of claim 1, wherein the sequencing is at a depth of about 20× to about 100×.

26. The method of claim 1, further comprising sequencing the plurality of polynucleotides to obtain the plurality of sequences.

27. A system comprising:
a computing node comprising a computer readable storage medium having program instructions embodied therewith, the program instructions executable by a processor of the computing node to cause the processor to perform a method comprising:
sequencing the plurality of polynucleotides to obtain a plurality of sequences;
providing the plurality of sequences to a trained classifier, the trained classifier adapted to accept a plurality of sequences and output a class label indicative of the presence of a somatic variant within the plurality of sequences;
receiving from the trained classifier a class label indicative of the presence of a somatic variant within the plurality of sequences.

28. The system of claim 27, the method further comprising:
based on the label, indicating whether a cancer clone is present in the sample.

29. The system of claim 27, the method further comprising:
based on the label, indicating whether a clonal expansion is present in the sample.

30. The system of claim 27, wherein the label has an associated probability.

31. The system of claim 27, the method further comprising:
providing clinical data to the trained classifier, the clinical data being related to an originator of the sample.

32. The system of claim 27, wherein the clinical data comprises an indication of smoking by the originator of the sample.

33. The system of claim 27, wherein the clinical data comprises family history of the originator of the sample.

34. The system of claim 27, the method further comprising:
providing population data to the trained classifier, the population data being related to an originator of the sample.

35. The system of claim 27, wherein the plurality of sequences comprise a plurality of somatic variants.

36. The system of claim 27, wherein the trained classifier comprises an artificial neural network.

37. The system of claim 27, wherein the trained classifier comprises a regression model.

38. The system of claim 27, wherein the trained classifier comprises a random decision forest.

39. The system of claim 27, wherein the trained classifier comprises an SVM.

40. The system of claim 36, wherein the artificial neural network is a convolutional neural network.

41. The system of claim 36, wherein the artificial neural network is a recurrent neural network.

42. The system of claim 27, wherein the sample comprises blood.

43. The system of claim 27, wherein the sample comprises cerebrospinal fluid.

44. The system of claim 27, wherein the plurality of polynucleotides comprises DNA.

45. The system of claim 27, wherein the plurality of polynucleotides comprises methylated DNA.

46. The system of claim 27, the method further comprising:
providing fragment lengths of the plurality of sequences to the trained classifier.

47. The system of claim 27, wherein the plurality of polynucleotides comprises RNA.

48. The system of claim 27, wherein the sequencing is at a depth of 100× or less.

49. The system of claim 27, wherein the sequencing is at a depth of 85× or less.

50. The system of claim 27, wherein the sequencing is at a depth of about 20× to about 85×.

51. The system of claim 27, wherein the sequencing is at a depth of about 20× to about 100×.

52. The system of claim 27, further comprising sequencing the plurality of polynucleotides to obtain the plurality of sequences.

53. A computer program product for analyzing a sample comprising a plurality of polynucleotides, the computer program product comprising a computer readable storage medium having program instructions embodied therewith, the program instructions executable by a processor to cause the processor to perform a method comprising:
sequencing the plurality of polynucleotides to obtain a plurality of sequences;
providing the plurality of sequences to a trained classifier, the trained classifier adapted to accept a plurality of sequences and output a class label indicative of the presence of a somatic variant within the plurality of sequences;
receiving from the trained classifier a class label indicative of the presence of a somatic variant within the plurality of sequences.

54. The computer program product of claim 53, the method further comprising:
based on the label, indicating whether a cancer clone is present in the sample.

55. The computer program product of claim 53, the method further comprising:
based on the label, indicating whether a clonal expansion is present in the sample.

56. The computer program product of claim 53, wherein the label has an associated probability.

57. The computer program product of claim 53, the method further comprising:
providing clinical data to the trained classifier, the clinical data being related to an originator of the sample.

58. The computer program product of claim 53, wherein the clinical data comprises an indication of smoking by the originator of the sample.

59. The computer program product of claim 53, wherein the clinical data comprises family history of the originator of the sample.

60. The computer program product of claim 53, the method further comprising:
providing population data to the trained classifier, the population data being related to an originator of the sample.

61. The computer program product of claim 53, wherein the plurality of sequences comprise a plurality of somatic variants.

62. The computer program product of claim 53, wherein the trained classifier comprises an artificial neural network.

63. The computer program product of claim 53, wherein the trained classifier comprises a regression model.

64. The computer program product of claim 53, wherein the trained classifier comprises a random decision forest.

65. The computer program product of claim 53, wherein the trained classifier comprises an SVM.

66. The computer program product of claim 62, wherein the artificial neural network is a convolutional neural network.

67. The computer program product of claim 62, wherein the artificial neural network is a recurrent neural network.

68. The computer program product of claim 53, wherein the sample comprises blood.

69. The computer program product of claim 53, wherein the sample comprises cerebrospinal fluid.

70. The computer program product of claim 53, wherein the plurality of polynucleotides comprises DNA.

71. The computer program product of claim 53, wherein the plurality of polynucleotides comprises methylated DNA.

72. The computer program product of claim 53, the method further comprising:
providing fragment lengths of the plurality of sequences to the trained classifier.

73. The computer program product of claim 53, wherein the plurality of polynucleotides comprises RNA.

74. The computer program product of claim 53, wherein the sequencing is at a depth of 100× or less.

75. The computer program product of claim 53, wherein the sequencing is at a depth of 85× or less.

76. The computer program product of claim 53, wherein the sequencing is at a depth of about 20× to about 85×.

77. The computer program product of claim 53, wherein the sequencing is at a depth of about 20× to about 100×.

78. The computer program product of claim 53, further comprising sequencing the plurality of polynucleotides to obtain the plurality of sequences.

79. A method of analyzing a sample comprising a plurality of polynucleotides, the method comprising:
reading at least one prior sequence of a polynucleotide associated with a tumor genome;
fitting a generative model to the at least one prior sequence;
receiving a plurality of sequences of the plurality of polynucleotides;
applying the generative model to the plurality of sequences to determine a probability that a first somatic clone is present in the plurality of sequences;
based on the probability, determining a label indicative of the presence of the first somatic clone in the sample.

80. The method of claim 79, wherein the label has an associated probability.

81. The method of claim 79, wherein the generative model comprises a linear-Gaussian model.

82. The method of claim 79, wherein the generative model comprises a linear-negative binomial model.

83. The method of claim 79, wherein the generative model comprises a latent factor model.

84. The method of claim 79, wherein the generative model comprises a factor analysis model.

85. The method of claim 79, further comprising
inferring a phylogenetic tree from the at least one prior sequence.

86. The method of claim 79, further comprising:
updating the generative model based on the plurality of sequences.

87. The method of claim 86, wherein the at least one prior sequence comprises a second somatic clone, the method further comprising:
based on the updated generative model, determining a probability that the sample contains the second somatic clone.

88. The method of claim 86, wherein the at least one prior sequence comprises a second somatic clone, the method further comprising:
based on the updated generative model, determining a probability that the sample contains a descendent of the second somatic clone.

89. The method of claim 86, wherein the at least one prior sequence comprises a second somatic clone, the method further comprising:
based on the updated generative model, determining a probability that the sample contains a third somatic clone related to the second somatic clone within the phylogenetic tree.

90. The method of claim 86, further comprising:
based on the updated generative model, determining a probability that the sample shares at least one somatic mutation with the at least one prior sequence.

91. The method of claim 86, further comprising:
based on the updated generative model, determining a probability that the sample shares at least one clonal expansion with the at least one prior sequence.

92. The method of claim 79, further comprising:
based on the label, indicating whether a cancer clone is present in the sample.

93. The method of claim 79, further comprising:
based on the label, indicating whether a clonal expansion is present in the sample.

94. The method of claim 79, wherein the label has an associated probability.

95. The method of claim 79, wherein the plurality of sequences comprise a plurality of somatic variants.

96. The method of claim 79, wherein the sample comprises blood.

97. The method of claim 79, wherein the sample comprises cerebrospinal fluid.

98. The method of claim 79, wherein the plurality of polynucleotides comprises DNA.

99. The method of claim 79, wherein the plurality of polynucleotides comprises methylated DNA.

100. The method of claim 79, wherein the plurality of polynucleotides comprises RNA.

101. The method of claim 79, wherein the sequencing is at a depth of 100× or less.

102. The method of claim 79, wherein the sequencing is at a depth of 85× or less.

103. The method of claim 79, wherein the sequencing is at a depth of about 20× to about 85×.

104. The method of claim 79, wherein the sequencing is at a depth of about 20× to about 100×.

105. The method of claim 79, further comprising sequencing the plurality of polynucleotides to obtain the plurality of sequences.

106. A system comprising:
a computing node comprising a computer readable storage medium having program instructions embodied therewith, the program instructions executable by a processor of the computing node to cause the processor to perform a method comprising:
reading at least one prior sequence of a polynucleotide associated with a tumor genome;
fitting a generative model to the at least one prior sequence;
receiving a plurality of sequences of the plurality of polynucleotides;
applying the generative model to the plurality of sequences to determine a probability that a first somatic clone is present in the plurality of sequences;
based on the probability, determining a label indicative of the presence of the first somatic clone in the sample.

107. The system of claim 106, wherein the label has an associated probability.

108. The system of claim 106, wherein the generative model comprises a linear-Gaussian model.

109. The system of claim 106, wherein the generative model comprises a linear-negative binomial model.

110. The system of claim 106, wherein the generative model comprises a latent factor model.

111. The system of claim 106, wherein the generative model comprises a factor analysis model.

112. The system of claim 106, the method further comprising
inferring a phylogenetic tree from the at least one prior sequence.

113. The system of claim 106, the method further comprising:
updating the generative model based on the plurality of sequences.

114. The system of claim 113, wherein the at least one prior sequence comprises a second somatic clone, the method further comprising:
based on the updated generative model, determining a probability that the sample contains the second somatic clone.

115. The system of claim 113, wherein the at least one prior sequence comprises a second somatic clone, the method further comprising:
based on the updated generative model, determining a probability that the sample contains a descendent of the second somatic clone.

116. The system of claim 113, wherein the at least one prior sequence comprises a second somatic clone, the method further comprising:
based on the updated generative model, determining a probability that the sample contains a third somatic clone related to the second somatic clone within the phylogenetic tree.

117. The system of claim 113, further comprising:
based on the updated generative model, determining a probability that the sample shares at least one somatic mutation with the at least one prior sequence.

118. The system of claim 113, further comprising:
based on the updated generative model, determining a probability that the sample shares at least one clonal expansion with the at least one prior sequence.

119. The system of claim 106, the method further comprising:
based on the label, indicating whether a cancer clone is present in the sample.

120. The system of claim 106, the method further comprising:
based on the label, indicating whether a clonal expansion is present in the sample.

121. The system of claim 106, wherein the label has an associated probability.

122. The system of claim 106, wherein the plurality of sequences comprise a plurality of somatic variants.

123. The system of claim 106, wherein the sample comprises blood.

124. The system of claim 106, wherein the sample comprises cerebrospinal fluid.

125. The system of claim 106, wherein the plurality of polynucleotides comprises DNA.

126. The system of claim 106, wherein the plurality of polynucleotides comprises methylated DNA.

127. The system of claim 106, wherein the plurality of polynucleotides comprises RNA.

128. The system of claim 106, wherein the sequencing is at a depth of 100× or less.

129. The system of claim 106, wherein the sequencing is at a depth of 85× or less.

130. The system of claim 106, wherein the sequencing is at a depth of about 20× to about 85×.

131. The system of claim 106, wherein the sequencing is at a depth of about 20× to about 100×.

132. The system of claim 106, further comprising sequencing the plurality of polynucleotides to obtain the plurality of sequences.

133. A computer program product for analyzing a sample comprising a plurality of polynucleotides, the computer program product comprising a computer readable storage medium having program instructions embodied therewith, the program instructions executable by a processor to cause the processor to perform a method comprising:
reading at least one prior sequence of a polynucleotide associated with a tumor genome;
fitting a generative model to the at least one prior sequence;
receiving a plurality of sequences of the plurality of polynucleotides;
applying the generative model to the plurality of sequences to determine a probability that a first somatic clone is present in the plurality of sequences;
based on the probability, determining a label indicative of the presence of the first somatic clone in the sample.

134. The computer program product of claim 133, wherein the label has an associated probability.

135. The computer program product of claim 133, wherein the generative model comprises a linear-Gaussian model.

136. The computer program product of claim 133, wherein the generative model comprises a linear-negative binomial model.

137. The computer program product of claim 133, wherein the generative model comprises a latent factor model.

138. The computer program product of claim 133, wherein the generative model comprises a factor analysis model.

139. The computer program product of claim 133, the method further comprising inferring a phylogenetic tree from the at least one prior sequence.

140. The computer program product of claim 133, the method further comprising:
updating the generative model based on the plurality of sequences.

141. The computer program product of claim 140, wherein the at least one prior sequence comprises a second somatic clone, the method further comprising:
based on the updated generative model, determining a probability that the sample contains the second somatic clone.

142. The computer program product of claim 140, wherein the at least one prior sequence comprises a second somatic clone, the method further comprising:
based on the updated generative model, determining a probability that the sample contains a descendent of the second somatic clone.

143. The computer program product of claim 140, wherein the at least one prior sequence comprises a second somatic clone, the method further comprising:
based on the updated generative model, determining a probability that the sample contains a third somatic clone related to the second somatic clone within the phylogenetic tree.

144. The computer program product of claim 140, further comprising:
based on the updated generative model, determining a probability that the sample shares at least one somatic mutation with the at least one prior sequence.

145. The computer program product of claim 140, further comprising:
based on the updated generative model, determining a probability that the sample shares at least one clonal expansion with the at least one prior sequence.

146. The computer program product of claim 133, the method further comprising:
based on the label, indicating whether a cancer clone is present in the sample.

147. The computer program product of claim 133, the method further comprising:
based on the label, indicating whether a clonal expansion is present in the sample.

148. The computer program product of claim 133, wherein the label has an associated probability.

149. The computer program product of claim 133, wherein the plurality of sequences comprise a plurality of somatic variants.

150. The computer program product of claim 133, wherein the sample comprises blood.

151. The computer program product of claim 133, wherein the sample comprises cerebrospinal fluid.

152. The computer program product of claim 133, wherein the plurality of polynucleotides comprises DNA.

153. The computer program product of claim 133, wherein the plurality of polynucleotides comprises methylated DNA.

154. The computer program product of claim 133, wherein the plurality of polynucleotides comprises RNA.

155. The computer program product of claim 133, wherein the sequencing is at a depth of 100× or less.

156. The computer program product of claim 133, wherein the sequencing is at a depth of 85× or less.

157. The computer program product of claim 133, wherein the sequencing is at a depth of about 20× to about 85×.

158. The computer program product of claim 133, wherein the sequencing is at a depth of about 20× to about 100×.

159. The computer program product of claim 133, further comprising sequencing the plurality of polynucleotides to obtain the plurality of sequences.

160. A method of analyzing a sample comprising a plurality of polynucleotides, the method comprising:
receiving a plurality of sequences of the plurality of polynucleotides;
identifying one or more inherited variant and one or more somatic variant among the plurality of sequences;
providing the one or more inherited variant to a first trained classifier;
providing the one or more somatic variant to a second trained classifier;
determining by the first and second trained classifier the presence of aneuploidy in the plurality of polynucleotides.

161. The method of claim 160, wherein the plurality of sequences comprise a plurality of somatic variants.

162. The method of claim 160, wherein the first or second trained classifier comprises an artificial neural network.

163. The method of claim 160, wherein the first or second trained classifier comprises a regression model.

164. The method of claim 160, wherein the first or second trained classifier comprises a random decision forest.

165. The method of claim 160, wherein the first or second trained classifier comprises an SVM.

166. The method of claim 162, wherein the artificial neural network is a convolutional neural network.

167. The method of claim 162, wherein the artificial neural network is a recurrent neural network.

168. The method of claim 160, wherein the sample comprises blood.

169. The method of claim 160, wherein the sample comprises cerebrospinal fluid.

170. The method of claim 160, wherein the plurality of polynucleotides comprises DNA.

171. The method of claim 160, wherein the plurality of polynucleotides comprises methylated DNA.

172. The method of claim 160, wherein the plurality of polynucleotides comprises RNA.

173. The method of claim 160, wherein the sequencing is at a depth of 100× or less.

174. The method of claim 160, wherein the sequencing is at a depth of 85× or less.

175. The method of claim 160, wherein the sequencing is at a depth of about 20× to about 85×.

176. The method of claim 160, wherein the sequencing is at a depth of about 20× to about 100×.

177. The method of claim 160, further comprising sequencing the plurality of polynucleotides to obtain the plurality of sequences.

178. A system comprising:
a computing node comprising a computer readable storage medium having program instructions embodied therewith, the program instructions executable by a processor of the computing node to cause the processor to perform a method comprising:
receiving a plurality of sequences of the plurality of polynucleotides;
identifying one or more inherited variant and one or more somatic variant among the plurality of sequences;
providing the one or more inherited variant to a first trained classifier;
providing the one or more somatic variant to a second trained classifier;
determining by the first and second trained classifier the presence of aneuploidy in the plurality of polynucleotides.

179. The system of claim 178, wherein the plurality of sequences comprise a plurality of somatic variants.

180. The system of claim 178, wherein the first or second trained classifier comprises an artificial neural network.

181. The system of claim 178, wherein the first or second trained classifier comprises a regression model.

182. The system of claim 178, wherein the first or second trained classifier comprises a random decision forest.

183. The system of claim 178, wherein the first or second trained classifier comprises an SVM.

184. The system of claim 180, wherein the artificial neural network is a convolutional neural network.

185. The system of claim 180, wherein the artificial neural network is a recurrent neural network.

186. The system of claim 178, wherein the sample comprises blood.

187. The system of claim 178, wherein the sample comprises cerebrospinal fluid.

188. The system of claim 178, wherein the plurality of polynucleotides comprises DNA.

189. The system of claim 178, wherein the plurality of polynucleotides comprises methylated DNA.

190. The system of claim 178, wherein the plurality of polynucleotides comprises RNA.

191. The system of claim 178, wherein the sequencing is at a depth of 100× or less.

192. The system of claim 178, wherein the sequencing is at a depth of 85× or less.

193. The system of claim 178, wherein the sequencing is at a depth of about 20× to about 85×.

194. The system of claim 178, wherein the sequencing is at a depth of about 20× to about 100×.

195. The system of claim 178, further comprising sequencing the plurality of polynucleotides to obtain the plurality of sequences.

196. A computer program product for analyzing a sample comprising a plurality of polynucleotides, the computer program product comprising a computer readable storage medium having program instructions embodied therewith, the program instructions executable by a processor to cause the processor to perform a method comprising:
receiving a plurality of sequences of the plurality of polynucleotides;
identifying one or more inherited variant and one or more somatic variant among the plurality of sequences;
providing the one or more inherited variant to a first trained classifier;
providing the one or more somatic variant to a second trained classifier;
determining by the first and second trained classifier the presence of aneuploidy in the plurality of polynucleotides.

197. The computer program product of claim 196, wherein the plurality of sequences comprise a plurality of somatic variants.

198. The computer program product of claim 196, wherein the trained classifier comprises an artificial neural network.

199. The computer program product of claim 196, wherein the trained classifier comprises a regression model.

200. The computer program product of claim 196, wherein the trained classifier comprises a random decision forest.

201. The computer program product of claim 196, wherein the trained classifier comprises an SVM.

202. The computer program product of claim 198, wherein the artificial neural network is a convolutional neural network.

203. The computer program product of claim 198, wherein the artificial neural network is a recurrent neural network.

204. The computer program product of claim 196, wherein the sample comprises blood.

205. The computer program product of claim 196, wherein the sample comprises cerebrospinal fluid.

206. The computer program product of claim 196, wherein the plurality of polynucleotides comprises DNA.

207. The computer program product of claim 196, wherein the plurality of polynucleotides comprises methylated DNA.

208. The computer program product of claim 196, wherein the plurality of polynucleotides comprises RNA.

209. The computer program product of claim 196, wherein the sequencing is at a depth of 100× or less.

210. The computer program product of claim 196, wherein the sequencing is at a depth of 85× or less.

211. The computer program product of claim 196, wherein the sequencing is at a depth of about 20× to about 85×.

212. The computer program product of claim 196, wherein the sequencing is at a depth of about 20× to about 100×.

213. The computer program product of claim 196, further comprising sequencing the plurality of polynucleotides to obtain the plurality of sequences.
